SEQUENCE LISTING (10 sequences)

SEQ ID NO: 1 - DNA, 18 bp, Artificial Sequence
  Synthetic primer hGAPDH_1F
aagggcatcctgggctac

SEQ ID NO: 2 - DNA, 20 bp, Artificial Sequence
  Synthetic primer hGAPDH_1R
aggggagattcagtgtggtg

SEQ ID NO: 3 - DNA, 20 bp, Artificial Sequence
  Synthetic primer hTRPV3_2_F
gtcttgaggagcagggagag

SEQ ID NO: 4 - DNA, 20 bp, Artificial Sequence
  Synthetic primer hTRPV3_2_R
caacccagtcacagcagaag

SEQ ID NO: 5 - DNA, 22 bp, Artificial Sequence
  Synthetic primer TRPC1_2_F
ccttctgttagtggctttttgc

SEQ ID NO: 6 - DNA, 21 bp, Artificial Sequence
  Synthetic primer TRPC1_2_R
gcctacatttgctggtcttca

SEQ ID NO: 7 - DNA, 27 bp, Artificial Sequence
  Synthetic primer TRPA1_3_F
gagagtccttcctagaaccatatctga

SEQ ID NO: 8 - DNA, 25 bp, Artificial Sequence
  Synthetic primer TRPA1_3_R
catgaggacaattgggacaaatatt

SEQ ID NO: 9 - DNA, 19 bp, Artificial Sequence
  Synthetic primer  TRPV1_F
gtttgggggtgttggtgtt

SEQ ID NO: 10 - DNA, 20 bp, Artificial Sequence
  Synthetic primer TRPV1_R
cctttgggatgtggttctgt

(19) United States
(12) Patent Application Publication
(10) Pub. No.: US 2016/0051493 A1
(43) Pub. Date: Feb. 25, 2016

(54) METHODS OF TREATMENT OF KERATINOCYTE-DERIVED LESIONS

(71) Applicants: Ellen A. Lumpkin, New York, NY (US); David M. Owens, Norwalk, CT (US); Yalda Moayedi-Esfahani, New York, NY (US); Aislyn Nelson, Montclair, NJ (US)

(72) Inventors: Ellen A. Lumpkin, New York, NY (US); David M. Owens, Norwalk, CT (US); Yalda Moayedi-Esfahani, New York, NY (US); Aislyn Nelson, Montclair, NJ (US)

(73) Assignee: THE TRUSTEES OF COLUMBIA UNIVERSITY IN THE CITY OF NEW YORK, New York, NY (US)

(21) Appl. No.: 14/716,865

(22) Filed: May 19, 2015

Related U.S. Application Data
(63) Continuation-in-part of application No. PCT/US13/70833, filed on Nov. 19, 2013.
(60) Provisional application No. 61/728,210, filed on Nov. 19, 2012.
(60) Provisional application No. 62/145,669, filed on Apr. 10, 2015.

(51) Int. Cl. A61K 31/125; A61K 31/69; C12Q 1/68; A61K 8/35; A61K 8/58; A61Q 17/04; A61K 9/00

(52) U.S. Cl. CPC A61K 31/125; A61Q 17/04; A61K 31/69; A61K 9/0014; A61K 8/35; A61K 8/58; C12Q 1/6886; A61K 2800/74; C12Q 2600/158

(57) ABSTRACT

Methods are provided for diagnosing and treating or preventing keratinocyte-derived lesions, e.g., SCC, (including high-risk forms), non-melanoma skin cancers (including high-risk forms), and actinic keratinosis (“AK”) by administering a therapeutically effective amount of camphor oil, camphor oil derivatives or components, and certain terpene TRPV3 agonists including 2-APB.

### CROSS-REFERENCE TO RELATED APPLICATIONS

The present application is a continuation-in-part of international application Serial No. PCT/US13/70833, entitled “Methods of Treatment of Keratinocyte-Derived Lesions” and filed Nov. 19, 2013, which claims priority to U.S. Provisional Application No. 61/728,210, filed Nov. 19, 2012, entitled, “Methods of Treatment of Keratinocyte-Derived Lesions,” and which claims benefit to U.S. Provisional Application No. 62/145,669, filed Apr. 10, 2015, entitled, “Methods of Treatment of Keratinocyte Derived Lesions, the entire contents of which are hereby incorporated by reference as if fully set forth herein, under 35 U.S.C. §119(e).

### STATEMENT OF GOVERNMENTAL INTEREST

This invention was made with Government support under Contract No. NIH-NIAMS R01AR051219 awarded by the National Institutes of Health National Institute of Arthritis and Musculoskeletal and Skin Diseases. The Government has certain rights in the invention. This invention was also made with government support under Contract No P30ES009089 awarded by the National Institutes of Health National Institute of Environmental Health Sciences. The government has certain rights in the invention.

### BACKGROUND

Squamous cell carcinoma (SCC) is the second most common form of non-melanoma skin cancer in the United States. A significant subset of high-risk forms of SCCs is highly invasive and metastatic. These SCCs are associated with a comparatively high risk of recurrence, resulting in significant mortality. SCC can be diagnosed by biopsy; however, current diagnostic biomarkers cannot distinguish high-risk SCCs that are likely to become metastatic from those that can be effectively treated with surgery alone. Current methods of treatment, i.e. surgery, radiotherapy, and chemotherapy, require continued monitoring due to the metastatic nature of the disease.

To date, there is no accepted system for defining high-risk SCC. The potential for advanced or aggressive disease can be attributed to a combination of tumor factors and host factors. Most high-risk tumors will have more than one risk factor present. As prognostic models have not yet been developed, it is unknown how various combinations of risk factors impact risk of recurrence or metastasis. Thus, it remains difficult to estimate these risks for an individual patient and make reasonable treatment recommendations. Development of reliable prognostic models will greatly aid treatment decisions in SCC. There is also a great need for new methods of treating or preventing SCC and other keratinocyte-derived lesions, both pre-cancerous and cancerous.

### SUMMARY

In certain embodiments, methods are provided for treating keratinocyte-derived lesions, e.g., SCC (including high-risk forms), non-melanoma skin cancers (including high-risk forms), and actinic keratinosis (“AK”) by administering to the lesion a therapeutically effective amount of an active agent as described which include terpenoid TRPV3 agonists, camphor oil or a terpene constituent of camphor oil, or a camphor oil derivative. Preferred agents include camphor oil, camphor, alpha-pinene, dipentene and 2-aminoethoxydiphenyle borate (2-APB). Embodiments are also directed to pharmaceutical compositions comprising one or more active agents, preferably formulated for topical application.

Embodiments of the invention include methods for treating a keratinocyte-derived lesion, including non-melanoma skin cancers such as squamous cell carcinoma and AK, by administering to the lesion a therapeutically effective amount of one or more TRPV3 agonists, thereby treating or preventing the lesion. Such agonists include camphor or 2-APB and also one or more TRPV3 agonists selected from the group comprising camphor, 2-APB, (+)-Borneol, (−)-Isopinocampheol, (−)-Fenchone, (−)-Trans-pinocarveol, Isoborneol, (+)-Camphorquinone, (−)-a-Thujone, 6-tert-butyl-m-cresol, Carvacrol, Thymol, p-xylenol, Kreosol, Propofol, Dihydrocarveol, (−)-Carveol, (−)-Isopulegol, and (+)-Linalool or a biologically active derivative thereof. In certain embodiments the TRPV3 agonist is applied to the lesion before it is surgically removed or it is applied to the affected area from which the lesion was surgically removed, or both.

Other embodiments are directed to methods wherein a subject identified as having actinic keratosis or at risk of developing actinic keratosis; is treated by administering to an affected area a therapeutically effective amount of a TRPV3 agonist, including camphor or 2-APB, thereby treating or preventing the actinic keratosis. In an embodiment the TRPV3 agonist is applied to the affected area before actinic keratosis is surgically removed, or after surgery to the affected area from which the actinic keratosis was surgically removed or both.

In yet another embodiment methods are provided for diagnosing high risk non-melanoma skin cancer by obtaining a biopsy of a non-melanoma skin cancer from a subject; obtaining a control biopsy either from a normal subject not afflicted with cancer, or a matched-sample from a non-affected area from subject; determining the level of TRPV3 mRNA in the subject biopsy and the level of TRPV3 in the control biopsy; and diagnosing the non-melanoma skin cancer as a high-risk form if the level of TRPV3 mRNA in the squamous cell carcinoma biopsy is either significantly higher or significantly lower than the level in the control biopsy. In an embodiment once a diagnosis of a high-risk form of cancer is made, then the subject is given aggressive treatment, for example surgery to remove the high-risk cancer in combination with application of therapeutically effective amounts of one or more TRPV3 agonists to the cancer before removal and to the affected area after it is removed. In some embodiments the subject is an immunocompromised patient such as an organ transplant patient.

Other embodiments are directed to pharmaceutical compositions comprising therapeutically effective amounts of camphor in a range of from about from 0.0608%-99.5%, or 2-APB in a range of from about 0.000056-1% or a combination of both formulated for topical application or microinjection or formulated into liposomes.

Another embodiment is directed to a pharmaceutical composition comprising therapeutically effective amounts of one or more TRPV3 agonists selected from the group comprising: camphor, 2-APB, (+)-Borneol, (−)-Isopinocampheol, (−)-Fenchone, (−)-Trans-pinocarveol, Isoborneol, (+)-Camphorquinone, (−)-a-Thujone, 6-tert-butyl-m-cresol, Carvacrol, Thymol, p-xylenol, Kreosol, Propofol, Dihydrocarveol, (−)-Carveol, (−)-Isopulegol, and (+)-Linalool or derivatives thereof. In some embodiments the formulation is a sunscreen.

In some embodiments the keratinocyte-derived lesions are AK or precancerous, a benign skin tumor or a non-melanoma cancer (including high risk forms) including SCC and the lesion is treated with a therapeutically effective amount of any herein described active agent including terpenoid TRPV3 agonists, camphor oil, a constituent of camphor oil, or a camphor oil derivative. In preferred embodiments administration is topical. In some embodiments the TRPV3 agonists include camphor, 2-aminoethoxydiphenyle borate (2-APB), (+)-Borneol, (−)-Isopinocampheol, (−)-Fenchone, (−)-Trans-pinocarveol, Isoborneol, (+)-Camphorquinone, (−)-a-Thujone, 6-tert-butyl-m-cresol, Carvacrol, Thymol, p-xylenol, Kreosol, Propofol, Dihydrocarveol, (−)-Carveol, (−)-Isopulegol, and (+)-Linalool, alpha-pinene oxide, 1,8-Cineole, (−)-alpha-Pinene, Isobornyl acetate, Dihydrocarbeol, p-cymene, Carvacrol methylether, (−) methol, (−)-Carvone, (+)-Dihydrocarvone, (−)-Menthone, (+)-Limonene (dipentene), Terpineol, Geraniol, 1-Isopropyl-4-methyl-bicyclo[3.1.0]hexan-4-ol, (−)-alpha-Bisabolol, and mugetanol. Isopulegol, (−)-Menthol.

In other embodiments the therapeutic agent camphor oil or a camphor oil constituent or camphor oil derivative. as described herein. Certain camphor oil constituents include pinene, eucalyptol, camphene, β-pinene, sabinene, phellandrene, 1,8-cineole, γ-terpinene, p-cymene, terpinolene, furfural, camphor, linalool, bornyl acetate, terpinen-4-ol, caryophyllene, borneol, piperitone, geraniol, safrole, cinnamaldehyde, methyl cinnamate, eugenol, 4-ethyl-o-xylene, α-terpinene, α-phellandrene, 3-carene, 3,3,5-trimethylcyclohexanol, 4-isopropyl-2-cyclohexenone, 2,3-dimethyl thiphene, 2,5-dimethyl-3-hexyne-2,5-diolm, and α-pinene,

In some embodiments high risk nonmelanoma cancers are diagnosed by a method including (i) obtaining a biopsy of the nonmelanoma cancer from a subject; (ii) obtaining a control biopsy either from a normal subject not afflicted with non-melanoma cancer or from a matched-sample taken from a non-affected area from the subject; (iii) determining an expression level of mRNA encoding one or more TRP ion channels selected from the group consisting of: TRPV3, TRPV1, TRPC1, and TRPA1, in the subject biopsy and in the control biopsy; and (iv) diagnosing the nonmelanoma cancer as a high-risk form if the level of mRNA encoding the one or more of the ion channels in the subject biopsy is either significantly higher or significantly lower than the corresponding mRNA level in the control biopsy. The subject having a high risk cancer is then treated aggressively.

In some embodiments the therapeutic agent slows progression of a benign or precancerous lesion to a cancerous lesion, or promotes regression of a pre-cancerous or cancerous lesion. In some embodiments the agent is camphor oil, white camphor oil, or a camphor oil constituent or camphor oil derivative. In some embodiments the pharmaceutical composition of is formulated as a cosmetic.

In some embodiments high risk non-melanoma cancers are diagnosed by a method including (i) obtaining a biopsy of squamous cell carcinoma or non-melanoma cancer from a patient; (ii) obtaining a control biopsy either from a normal subject not afflicted with squamous cell carcinoma or non-melanoma cancer and not having an endogenous TRP mutation, or from a matched-sample taken from a non-affected area from the patient; (iii) determining an expression level of mRNA encoding one or more TRP ion channels selected from the group consisting of: TRPV3, TRPV1, TRPC1, and TRPA1, in the patient biopsy and in the control biopsy; and (iv) diagnosing the squamous cell carcinoma as a high-risk form of squamous cell carcinoma or the non-melanoma cancer as a high risk form of non-melanoma cancer if the level of mRNA encoding the one or more of the ion channels in the patient biopsy is either significantly higher or significantly lower than the corresponding mRNA level in the control biopsy. The subject having a high risk cancer is then treated aggressively.

### BRIEF DESCRIPTION OF THE DRAWINGS

The following figures form part of the present specification and are included to further demonstrate certain embodiments of the present invention. The invention may be better understood by reference to one or more of these drawings in combination with the detailed description of specific embodiments presented herein.

FIG. 1A-E. Cytoplasmic calcium signals elicited by TRP-channel agonists are potentiated in differentiating human keratinocytes. Responses to TRP-channel agonists (FIG. 1A-FIG. 1D) and gene expression levels (FIG. 1E) were compared in normal human epidermal keratinocytes cultured for 2-3 days in 0.06 mM Ca²⁺ (Growth; green) or in (Differentiated; blue). FIG. 1A-FIG. 1D: Live-cell Ca²⁺ imaging. FIG. 1A: Pseudocolor images depict fura-2 ratios of human keratinocytes at rest (left) and during acute application of saturating concentrations of two TRP channel agonists (10 mM Camphor; middle) and TRPV4 (3 μM 4αPDD; right). FIG. 1B: The percent increases in fluorescence ratio are plotted for representative individual keratinocytes (lines). Responding cells were identified as those showing a 20% increase in fluorescence ratio during agonist application. Symbols depict time points in FIG. 1A. FIG. 1C: Quantification of TRP-agonist-evoked responses (N=3 experiments with >300 cells per replicate, X²<0.0001). FIG. 1D: Responses to structurally unrelated TRPV3 agonists, camphor and 2-APB, were larger in differentiated human keratinocytes (N=3 experiments with >640 cells per replicate; symbol *P<0.05; two-way ANOVA). FIG. 1E: Quantitative PCR demonstrated that TRPV3 and keratinocyte differentiation markers (keratin-1, KRT1; loricrin, LOR; and filaggrin; FLG) were upregulated upon differentiation. Fold increase in transcript levels compared with growth conditions is plotted (N=3 experiments; ***P<0.0001, *P<0.05, Student's t test).

FIG. 2A-2B. TRP channel responses and expression in human keratinocytes. FIG. 2A: Fura-2 responses to TRPV1 specific agonist, 1 μM Capsaicin, and TRPA1 agonist, 300 μM mustard oil, in human keratinocytes (Capsaicin=2.3+/−2.6%, Mustard Oil=0.6+/−1.2%; N=3 experiments). These responses demonstrate that responses to TRPV1 and TRPA1 agonists are not significantly different from zero. Thus, these TRP channels are functionally expressed at lower levels than TRPV3 in human keratinocytes in vitro. FIG. 2B: Quantitative PCR demonstrated that TRPV1 and TRPC1 were upregulated upon differentiation. TRPA1 was only amplified in one out of three experimental replicates, demonstrating that this transcript is expressed at low levels compared with TRPV3, TRPV1 and TRPC1. TRPV3 expression is plotted for reference. Expression level relative to GAPDH levels is plotted for both growth and differentiation conditions (N=3 experiments; **P<0.0004, *P<0.05, Student's t test).

FIG. 3A-3F. Constitutive activation of TRPV3 arrests human keratinocyte proliferation and promotes differentiation. FIG. 3A-FIG. 3B: Quantification of DAPI-labeled keratinocytes cultured in range of 2-APB concentrations. Brightfield images (left) correspond to concentrations plotted at right. Percent change from vehicle-treated control cells is plotted (N=5 experiments, **P≦0.006, Student's t test). FIG. 3C: Proliferation assays (45-min EdU pulse) (EdU=5-ethynyl-2′-deoxyuridine) after 24 hours treatment with vehicle, 12.5 μM, or 50 μM 2-APB (top panel, N=5 independent experiments, *P<0.05, ***P≦0.0001, Student's two-tail t test) or after 24 h treatment with vehicle, or camphor at the indicated concentrations (lower panel; N=3 independent experiments, **P<0.006, Student's two-tail t test). FIG. 3D: Gene expression levels compared by qPCR (LOR, FLG, Involucrin: IVL, Transglutaminate 3: TGM3) in keratinocytes treated with vehicle (1% ethanol) or 50 μM 2-APB. Fold increase in transcript levels in 2-APB-treated compared with vehicle-treated keratinocyte is plotted (N=3 independent experiments; *P<0.02, **P≦0.006, ***P<0.0001, Student's t test). FIG. 3E Organotypic 3D human skin equivalents treated with vehicle (top panel) or 50 μM 2-APB (bottom panel). Mean number of nucleated epidermal cell layers is plotted (n=26-28 sections from two grafts per treatment, E=epidermal layer, D=dermis, ***P<0.0001). FIG. 3F: Mean number of nucleated epidermal cell layers is plotted (n=26-28 sections from two grafts per treatment, E=epidermal layer, D=dermis, ***P<0.0001).

FIG. 4A-4B. Inhibition of TRPV1 does not alter camphor effects on human keratinocytes. FIG. 4A: Quantification of DAPI-labeled keratinocytes cultured in range of camphor concentrations with or without 0.5-1 μM AMG 9810. Percent change from vehicle-treated control cells is plotted (N=3 experiments; Two-way ANOVA, camphor effect P=0.0004). FIG. 4B: Proliferation assays (45-min EdU pulse) after 24 hours treatment with vehicle, 2-8 mM, camphor with or without 0.5-1 μM AMG 9810. Percent EdU positive cells are plotted (N=3 experiments; Two-way ANOVA, camphor effect P=0.0004). In both assays, camphor+AMG-9810 was indistinguishable from camphor alone. These data indicate that camphor attenuates keratinocyte proliferation by acting on at target other than TRPV1.

FIG. 5A-FIG. 5E. Epidermal TRP channels are potential therapeutic targets for the treatment of human SCC. FIG. 5A: TRPV3 expression levels assessed by qPCR in high-risk SCC biopsies (purple patterns) and normal adult skin. Fold increase compared with normal adult skin is plotted (black; N=4 replicates per sample, ¥ P<0.05, # P<0.005, Student's t test). Plots depict means±SDs. FIG. 5B: Live-cell calcium imaging of TRP-channel activation in two SCC cell lines. Fura-2 fluorescence ratios of SCC cells at rest and during camphor application (10 mM) are shown. Right: quantification of camphor-evoked responses (N>200 cells per SCC cell line; ¥ P<0.05, Student's t test). FIG. 5C: Transcript levels monitored by qPCR in human SCC-derived cell lines, SCC-13 and SCC-39, and normal keratinocytes. Fold increase compared with normal keratinocytes is plotted (N=4 replicates; *P<0.0001, # P<0.005, ¥ P<0.05, Student's t test). Plots depict means±SDs. FIG. 5D: Organotypic human skin equivalents with SCC cells were treated with vehicle (SCC-39; upper right) or 50 μM 2-APB (SCC-39; lower right) for 14 days. FIG. 5E: Number of invading cells per 10× field (upper) and SCC layer thickness (lower) were quantified for SCC-39 and SCC-13 organotypic cultures (3 rafts per treatment, T=tumor, D=dermis, # P<0.005 for treatment, two-way ANOVA).

FIG. 6A-6E. TRP channel and SCC biomarker expression in SCC biopsies. FIG. 6A: TRPV1, FIG. 6B: TRPA1, FIG. 6C: TRPC1, FIG. 6D: CCDN1, and FIG. 6E: EGFR expression levels assessed by qPCR in high-risk SCC biopsies and normal adult skin. Fold increase compared with normal adult skin is plotted (N=4 replicates per sample, *P<0.0001, Student's t test). Plots depict Means+/−SDs.

FIG. 7. TRP channel expression in SCC cell lines. TRPV1, TRPA1 and TRPC1 expression levels assessed by qPCR in SCC cell lines SCC-13, SCC-39 and normal human keratinocytes. Fold increase compared with normal adult skin is plotted (N=4 replicates per sample, **P<0.009, ***P<0.0001, Student's t test). Plots depict Means+/−SDs.

FIG. 8A-8C. SCC skin equivalents show reduced tumor thickness when treated with 2-APB. Micrographs show organotypic human skin equivalents with FIG. 8A SCC-73, FIG. 8B SCC-39, and FIG. 8C SCC-13 cells treated with vehicle (upper) or 50 μM 2-APB (lower) for 14 days. These three SCC cell lines were derived from three independent, de-identified, human high-risk SCC tumors.

FIG. 9A-9B. SCC-13 skin equivalents show reduced tumor thickness when treated with 2-APB. Micrographs show organotypic human skin equivalents with SCC-13 cells treated with vehicle FIG. 9A or 50 μM 2-APB FIG. 9B for 14 days.

FIG. 10. Two-stage chemical carcinogenesis model (DMBA-TPA). A two-stage chemical carcinogenesis model (DMBA-TPA) was used to induce benign precancerous lesions and SCCs in adult female mice. Mice were randomly assigned to treatment groups control (acetone) or 20% camphor oil and (N=12 mice per group) matched for lesion burden immediately prior to the start of treatment (week 0). Lesions were quantified and cohorts were photographed weekly beginning two weeks before treatment.

FIG. 11. Total Number of Malignant SCCs in mice treated with 20% camphor oil or vehicle. Groups were compared with two-way ANOVA followed by Bonferroni post hoc analysis to assess significant differences between treatment groups at each time point (N=12 mice per group; *P<0.05; **P≦0.01). Significant effects included treatment group: F(15,265)=101.40, P<0.0001; treatment duration: F(15,265)=5.16, P<0.0001; interaction: F(15,265)=2.30, P=0.004.

FIG. 12. Example of regression of benign precancerous lesion in a camphor oil-treated mouse compared with an acetone (vehicle)-treated control animal. At week 0 in the camphor oil-treated mouse, 10 tumors and at week 7, 1 tumor compared at week 0 in the vehicle-treated control, 13 tumors and at week 7, 11 tumors.

FIG. 13. Total number of malignant SCCs in mice treated with 20% camphor oil or vehicle alone. Curves show Boltzmann fits (R²>0.93 for each fit), which differed significantly (P<0.0001, extra Sum of Squares F test). Two-way ANOVA showed a significant effect of treatment group [F(1,15)=10.20, P=0.006] and treatment duration [F(15,15)=2.74, P=0.030] (N=12 mice per group).

FIG. 14. Regression of early-stage SCC and precancerous lesions in a camphor-oil treated mouse. An advanced SCC (bracket), which appeared by treatment day 9, progressed to an experimental endpoint by 38 days of treatment. An early-stage SCC (arrow) and a precancerous lesions (arrowhead) on this mouse appeared to regress between treatment day 37 and 38.

FIG. 15. Hematoxylin and eosin (H&E) staining of paraffin sections from SCC tumors in control and camphor-oil treatment groups. Advanced SCCs showed neoplastic keratinocytes surrounding muscle tissue (arrowheads), indicating an invasive lesion. A regressed SCC tumor from the camphor-oil group (right) showed an intact muscle layer (brackets). The middle and right panels are imagines of the advanced and regressed SCCs in FIG. 14.

FIG. 16. Tumor incidence in mice treated with camphor oil or vehicle. Tumor incidence remained at 100% in control mice but decreased to as low as 50% in camphor oil-treated mice (N=12 mice per group). A two-way ANOVA showed a highly significant effect of treatment group on tumor incidence [F(1,15)=14.40, P=0.0018]. The effect of treatment day was not significant [F(15,15)=1.0, NS].

FIG. 17. Time to experimental endpoint in mice treated with camphor oil or vehicle alone (N=12 mice per group). After 13 weeks of treatment, 75% of control mice and 50% of camphor oil-treated mice reached an experimental endpoint. But Kaplan-Meier survival analysis indicated that endpoint curves did not differ significantly between treatment groups.

FIG. 18. Topical camphor upregulates levels of a keratinocytes terminal differentiation marker (K10) in vivo. FVB mice (adult females; N=3-4) were treated twice per day for five days with camphor diluted in vehicle (acetone) at the indicated concentrations). Protein levels were assayed by Western blotting, normalized to beta-tubulin levels, and means were compared with Student's t tests (one-way unpaired). P values for pairwise comparisons are indicated in the figure.

FIG. 19A-19C. Terpenes induce calcium signaling in human keratinocytes, using live-cell calcium imaging of cultured normal human epidermal keratinocytes. FIG. 19A: no change in cytoplasmic calcium was identified in vehicle-treated keratinocytes. FIG. 19B: Camphor oil at 0.15% induced calcium increases FIG. 19C: α-pinene at 0.15% induced calcium increases.

FIG. 20A-FIG. 20C. Topical camphor oil slowed cutaneous SCC progression and induced regression of tumors in vivo. FIG. 20A, FIG. 20B, and FIG. 20C: (bottom curve; 20% in vehicle, wt/wt) reduced tumor burden compared with vehicle controls (upper curve). FIG. 20A: rate of malignant conversion; FIG. 20B: # pre-malignant tumors; FIG. 20B: tumor incidence. Two-way ANOVA (N=12 per group) group effect: F(1,25)=43.2), F(1,284)=106.9 (FIG. 20B), F(1,23)=11.2. Bonferroni post hoc (FIG. 20A): Means±SEM; *P<0.05; **P≦0.01. See also FIGS. 10, 11, 13 and 16 which overlap.

FIG. 21. Photographs of mice illustrating daily treatment with topical camphor oil (CaO) for seven weeks causing reduction. See also FIG. 12.

FIG. 22A-22B. A pair of graphs showing that terpenes reduced skin tumors in vivo. Mice treated daily with topical terpenes differ in premalignant tumor numbers. FIG. 22A: Dipentene (upper) and FIG. 22B: α-pinene (lower) showed the most striking tumor reductions compared with vehicle. Two-way ANOVA indicated significant group effects: Mean±SEM, N=4-5 mice per group, Dipentene: p=0.02, α-pinene: p=0.007.

FIG. 23. Data showing that topical camphor oil and terpene treatment increases proliferation in normal keratinocytes in vivo. Mice treated with daily topical compounds (Mean±SEM, Student's t test; N=2-6 mice, p<0.05, **p<0.01, ***p<0.001).

FIG. 24. A series of photographs and bar graphs (Left to Right) showing three dimensional human organotypic skin rafts. Cells derived from epidermal keratinocytes (first photograph) and independent cSCC tumor biopsies (SCC13 & SCC39; second & third photographs) were grown on a fibroblast-collagen matrix. Tumor cells (T) proliferate and invade the dermis-like matrix (first bar graph), while epidermal cells (second bar graph) remain above the dermis. (Third photograph): Tumor thickness and invasion, estimated via quantitative histomorphometry, differ between cultures seeded with distinct cSCC cell lines (Mean±SEM, Student's t test; N=3 rafts; p<0.01). This is a model for future work. See also FIGS. 5 and 8.

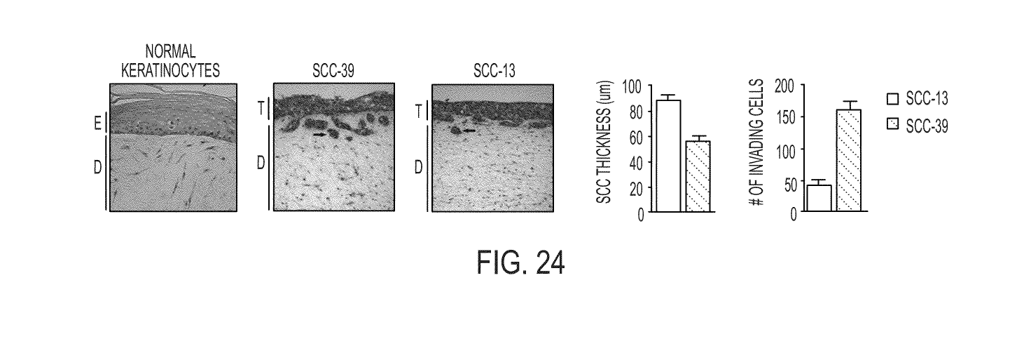

FIG. 25. A graph showing that proliferation with camphor oil treatment in healthy mouse skin is not TRPV3 dependent. The data shown in this figure indicate that in mice treated once a day for 5 consecutive days with 20% camphor oil show significant increases in proliferation compared to vehicle treated groups (One-way ANOVA, Bonferroni post-hoc WT-Acetone vs WT Camphor oil P<0.001; TrpV3 mutant-Acetone vs TrpV3-Camphor oil P<0.01). However, increases in proliferation were not significantly different between wild type and TRPV3 mutant mice (WT-Acetone vs TrpV3 mutant-Acetone not significant; WT-Camphor oil vs. TrpV3 mutant-Camphor oil not significant). Thus, this increase in proliferation was not TRPV3 dependent. N=4-5 mice per group.

FIG. 26A-26B. Data from a second cohort showing that topical camphor oil slows cSCC progression and induces regression of tumors in vivo. Tumors were induced using two-step chemical carcinogenesis and then mice were treated with 20% camphor white oil or acetone vehicle once daily. FIG. 26A: Fewer malignant SCCs formed with camphor oil treatment (lower curve) than with acetone vehicle treatment (upper curve). (Boltzmann sigmoidal fits, Camphor oil R²=0.98, V₅₀=8.56, Max=13.03; Vehicle R²=0.98, V₅₀=12.45, Max=26.28; F=(1,225)=169.27 P<0.0001 between groups). Two-way ANOVA (N=10 mice per group) group effect: F(1,25)=15.67 P=0.0006 (Malignant cSCCs), FIG. 26B: Pre-malignant lesions showed dramatic regression with camphor oil treatment (Lower curve) compared with vehicle treatment (upper curve). Note that the number of lesions per mouse in the control group falls off at 14 weeks because the number of mice with high tumor burden reached endpoint and were removed from the analysis. Bonferroni post hoc (Pre-malignant lesions): Means±SEM; *P<0.05; **P≦0.01.

FIG. 27A-27B. Dose-response studies were performed on mice treated daily with a concentration range of camphor oil. Tumors were induced using two-step chemical carcinogenesis and then mice were treated with acetone vehicle (0%) or a range of camphor oil concentrations (2.5%, 5%, 10%, 20%, or 40%). FIG. 27A: At 4 days of treatment, we found a significant reduction in premalignant tumors with 40% camphor oil treatment (**P<0.01,). At 25 days of treatment (FIG. 27B), we observed a significant reduction in tumors with 20% (*P<0.05) and 40% (***P<0.001) camphor oil treatments compared to the number of tumors at −4 days, whereas no significant reductions are found with 0%, 2.5%, 5%, or 10% camphor oil treatment. Plots show means±SD, N=4-5 mice per group. Curve fits: One-phase decay shows significant differences in dose-response relations between Day −4 and Day 25 of treatment.

### DETAILED DESCRIPTION

### 1. Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N.Y. (1990); Principles of Neural Science, 4th ed., Eric R. Kandel, James H. Schwartz, Thomas M. Jessell editors. McGraw-Hill/Appleton & Lange: New York, N. (2000). Unless defined otherwise, all technical and scientific terms used herein have the same meaning as commonly understood by one of ordinary skill in the art.

The term “actinic keratosis” (also called “solar keratosis” and “senile keratosis,” herein “AK”), as used herein, means a premalignant condition of thick, scaly, or crusty patches of skin. It is more common in fair-skinned people and it is associated with those who are frequently exposed to the sun, as it is usually accompanied by solar damage. AK is potentially pre-cancerous, since some progress to squamous cell carcinoma, so treatment is recommended. Untreated lesions have up to 20% risk of progression to squamous cell carcinoma. People who take immunosuppressive drugs, such as organ transplant patients, are 250 times more likely to develop actinic keratosis that may lead to skin cancer.

The term “activating a transient receptor potential vanilloid ion channel” (“TRPV channel”) as used herein, means increasing the proportion of receptors in an ion-conducting confirmation, which is expected to increase cation transport into the cell. This has the effect of reducing or arresting proliferation or normal and cancerous (e.g., SCC cells) human keratinocytes.

The term “active agent” as used herein collectively refers to terpene TRPV3 agonists including 2-APB (see Table 1), camphor oil, camphor oil derivatives, and components or constituents of camphor oil including camphor, and any other agent described herein for treatment or prevention of cancerous and precancerous keratinocyte-derived lesions, e.g., non-melanoma cancers including SCC (including high-risk forms of SCC and other non-melanoma skin cancers and actinic keratosis. Active agents also include any derivatives that retain the biological activity of treating a keratinocyte-derived lesion.

Active agents include (A)-(D): (A) “terpene TRPV3 agonists” as used herein and set forth in Table 1 comprise camphor, 2-aminoethoxydiphenyle borate (2-APB), (+)-Borneol, (−)-Isopinocampheol, (−)-Fenchone, (−)-Trans-pinocarveol, Isoborneol, (+)-Camphorquinone, (−)-a-Thujone, 6-tert-butyl-m-cresol, Carvacrol, Thymol, p-xylenol, Kreosol, Propofol, Dihydrocarveol, (−)-Carveol, (−)-Isopulegol, and (+)-Linalool, alpha-pinene oxide, 1,8-Cineole, (−)-alpha-Pinene, Isobornyl acetate, Dihydrocarbeol, p-cymene, Carvacrol methylether, (−) methol, (−)-Carvone, (+)-Dihydrocarvone, (−)-Menthone, (+)-Limonene (dipentene), Terpineol, Geraniol, 1-Isopropyl-4-methyl-bicyclo[3.1.0]hexan-4-ol, (−)-alpha-Bisabolol, mugetanol. (B) camphor oil, including camphor white oil from which camphor has been substantially removed by distillation, and constituents of camphor oil. Camphor oil is a complex mixture that includes various constituents. Two different lots of camphor white oil, were tested and the constituents are set forth in Table 2. Further constituents of camphor oil besides those in Table 2 include 1,8-cineole, Furfural, Linalool, Bornyl acetate, Terpinen-4-ol, Caryophyllene, Borneol, Piperitone, Geraniol, Safrole, Cinnamaldehyde, Methyl cinnamate, Eugenol. All of these are active agents for the purpose of embodiments of the invention. (C) Camphor white oil was used for most of the in vivo tumor experiments; the in vitro experiments with tumor cells were done with camphor and 2-APB. Camphor white oil constituents include: dipentene, α-pinene, eucalyptol, camphene, β-pinene, sabinene, phellandrene, 1,8-cineole, γ-terpinene, cymene, terpinolene, furfural, camphor, linalool, bornyl acetate, terpinen-4-ol, caryophyllene, borneol, piperitone, geraniol, safrole, cinnamaldehyde, methyl cinnamate, eugenol, 4-ethyl-o-xylene, α-terpinene, α-phellandrene, 3-carene, 3,3,5-trimethylcyclohexanol, 4-isopropyl-2-cyclohexenone, 2,3-dimethyl thiophene, 2,5-dimethyl-3-hexyne-2,5-diol, 2-carene. Of these, eucalyptol, dipentene (limonene), 4-ethyl-o-xylene, α-pinene, γ-terpinene, sabinene, camphene, and β-pinene, are some of the major components (data not shown). (D) Camphor derivatives include 4-methyl-benzylidene camphor (4-MBC), [3-(4′-methyl)benzylidene-bornan-2-one], 3-benzylidene camphor(3-benzylidene-bornan-2-one), polyacrylamidomethylbenzylidene camphor {N-[2(and 4)-2-oxyborn-3-ylidene-methyl)benzyl]acrylamide polymer}, trimonium-benzylidene camphor sulfate[3-(4′-trimethylammonium)-benzylidene-bornan-2-one methyl sulfate], terephthalydene dicamphorsulfonic acid [3,3′-(1,4-phenylenedimethine)-bis(7,7-dimethyl-2-oxo-bicyclo[2.2.1]hepta-ne-1-methanesulfonic acid} or salts thereof, and benzylidene camphorsulfonic acid [3-(4′-sulfo)benzylidenebornan-2-one] or salts thereof, 4-Methylbenzylidene camphor (4-MBC), Norcamphor. 4-Methylbenzylidene camphor. (4-MBC) is an organic camphor derivative that is used in the cosmetic industry for its ability to protect the skin against UV, specifically UV B radiation. As such it is used in sunscreen lotions and other skincare products claiming a SPF value. Its tradenames include Eusolex 6300 (Merck) and Parsol 5000 (DSM). Norcamphor is a chemical compound, classified as a ketone, which is an analog of camphor without the three methyl groups.

As used herein, “administering” an active agent may be performed using any of the various methods or delivery systems well known to those skilled in the art. The preferred method for administering camphor or derivatives thereof, camphor oil which contains other monoterpene constituents, or 2-APB at a dose that might be toxic systemically, is topical/transdermal administration directly to the keratinocyte-derived lesion. The administering can also be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchymally or microinjection.

As used herein “agonist” refers to molecules or compounds which mimic the action of a “native” or “natural” compound that activates one or more of the ion channels TRPV3, TRPV1, TRPA1, or TRPC1. Agonists may or may not be homologous to these natural compounds in respect to conformation, charge or other characteristics. In any event, regardless of whether the agonist is recognized in a manner similar to the “natural” ion channel, the agonist may cause physiologic and/or biochemical changes within the cell, such that the cell reacts to the presence of the agonist in the same manner as if the natural ion channel was present.

The terms “animal,” “patient,” or “subject” as used herein, mean any animal (e.g., mammals, (including, but not limited to humans, primates, dogs, cattle, cows, horses, kangaroos, pigs, sheep, goats, cats, rabbits, rodents, and transgenic non-human animals), and the like, which are to be the recipient of a particular treatment. Typically, the terms “animal” “subject” and “patient” are used interchangeably herein in reference to a human subject or a rodent. The preferred animal, patient, or subject is a human.

The term “an individual at risk” as used herein, means one may or may not have detectable disease, and may or may not have displayed detectable disease prior to the treatment methods described herein. “At risk” denotes that an individual who is determined to be more likely to develop a symptom based on conventional risk assessment methods or has one or more risk factors that correlate with development of SCC. An individual having one or more of these risk factors has a higher probability of developing SCC than an individual without these risk factors. Examples (i.e., categories) of risk groups are well known in the art and discussed herein.

The term “biomarker” as used herein, means any biological feature from an organism which is useful or potentially useful for measuring the initiation, progression, severity, pathology, aggressiveness, grade, activity, disability, mortality, morbidity, disease sub-classification or other underlying feature of one or more biological processes, pathogenic processes, diseases, or responses to therapeutic intervention. For the present invention, the biomarkers of high-risk SCC or high-risk non-melanoma skin cancer are one or more of the proteins TRPV3, TRPV1, TRPA1, and TRPC1, or biologically active fragment thereof.

The term “camphor” as used herein, means a terpenoid (it is a monoterpene) with the chemical formula C₁₀H₁₆O. It is an organic compound of the isoprenoid family that belongs to the group of bicyclic monoterpenes. A white, waxy solid with a penetrating, somewhat musty aroma, it is obtained from the wood of the camphor laurel (laurel family), Cinnamomum camphora (found in Asia), or produced synthetically from oil of turpentine. It exists in the optically active dextro and levo forms, and as the racemic mixture of the two forms. All of these melt within a degree of 178° C. (352° F.). The principal form is dextro-camphor, which occurs in the wood and leaves of the camphor tree (Cinnamomum camphora). Camphor is also synthesized commercially on a large scale from pinene, which yields mainly the racemic variety. Camphor is readily absorbed through the skin and produces substance feeling of warmth, and acts as a slight local anesthetic and anti-itch substance. There are anti-itch gels and soothing gels with camphor as the active ingredient. Camphor is an active ingredient (along with menthol) in vapor-steam products, such as Vicks VapoRub. The IUPAC name for camphor is 1,7,7 Trimethylbicyclo[2.2.1]heptan-2-one. Since 1983, the Federal Food and Drug Administration (FDA) have banned the sale of products with more than 11% camphor because it can be toxic if ingested.

The term, “camphor oil” as used herein means a colorless liquid obtained from the wood of the camphor tree (Cinnamomum camphora) by distillation and separation from the solid camphor, used in varnish, soaps, and shoe polish, and in medicine chiefly as a rubefacient. It is extracted from the wood by steam distillation. Many experiments described here were done with “camphor white oil” from which camphor has been substantially removed by distillation. Camphor oil may be a natural extract or a synthetic mixture of components (e.g., CAS number 8008-51-3). A natural extract or naturally derived camphor oil may have a different composition from lot to lot from that of a synthetic camphor oil. Terpenes other than camphor are major constituents of camphor oil. Several of these, including linalool, alpha-pinene, limonene (dipentene), and geraniol are TRPV3 agonists. In particular linalool has been reported to make up 90% of camphor oil in one study, and borneol is more effective than camphor as a TRPV3 agonist.

The term “camphor white oil” as used herein means the product of a distillation of camphor oil whereby camphor has been substantially removed.

The term “cancer” as used herein, includes the enumerated diseases which are any cancerous keratinocyte-derived lesions, e.g., a member of any class of SCC diseases, non-melanoma skin cancers. Cancer includes high-risk forms of SCC and high-risk non-melanoma skin cancers and neoplastic conditions, whether characterized as malignant, benign, soft tissue, or solid, and cancers of all stages and grades including pre- and post-metastatic cancers. SCC cancers that can appear on the skin, lips, mouth, lung, head, stomach, prostate, colon, rectum, throat, urinary tract, reproductive tract, and esophagus.

The term “precancerous lesion” includes any precancerous lesion, such as those found in actinic keratinosis (AK) characterized by the uncontrolled or aberrant growth of aberrant, though not malignant, keratinocytes. Precancerous lesions are also referred to in the literature often as a papillomas.

The term “derivative” of an active agent as used herein means any variation, deviation, change, or analog of the active agent, as defined herein. This may include, but is not limited to a variation in stereochemistry to either increase or decrease the size of a ring, or such as an addition or deletion of a substituent, or a variation in functional group, or an analog. Certain camphor oil and 2-APB derivatives or analogs are known in the art (e.g., Y, Dobrydneva et al., 2005).

The term “enumerated disease” as used herein means any a disease or condition have a cancerous or pre-cancerous keratinocyte-derived lesion including non-melanoma skin cancers including basal cell carcinoma, squamous cell carcinoma (including the high risk forms), angiosarcoma, cutaneous B-cell lymphoma, cutaneous T-cell lymphoma, dermatofibrosarcoma protuberans, merkel cell carcinoma, sebaceous gland carcinoma, and other cancers that spread from other areas of the body to the skin, such as breast cancer and mouth cancers, and actinic keratosis.

The “high-risk” form of Squamous Cell Carcinoma (SCC) is meant as a subset of SCC that are highly invasive and metastatic, which compared to low-risk lesions exhibit an increased rate of recurrence, resulting in significant mortality. Surgery alone is not enough to treat these high-risk SCCs, so adjuvant radiotherapy and chemotherapy are given with continued monitoring due to the metastatic nature of the disease. In the general literature prior to the discoveries described herein, a diagnosis of high-risk SCC was based entirely on a battery of clinical and histological criteria because there was no known biological marker. In general a surgeon refers to a high-risk SCC as one that that has a greater risk for recurrence (following treatment) and metastasis, based on the following criteria:

Clinical Features:
1. Size >2 cm;
2. Anatomic site (particularly SCCs of lip and ear, other high-risk sites include eyelids, nose, mucous membranes, scalp/forehead/temple, anogenital region);
3. Rapid growth;
4. Tumors arising in injured or chronically diseased/inflamed skin;
5. Immunosuppression;
6. History of irradiation to skin;
7. History of recurrence following previous treatment.

### Histologic Features:

1. Tumor depth >4 mm
2. Poorly differentiated histology
3. Perineural invasion

The high-risk form of SCC and the high-risk forms of other non-melanoma cancers herein described have now been discovered to be dysregulated for TRP ion channel expression so that they can be diagnosed if they expresses either significantly higher or significantly lower than normal levels of one or more of the proteins of TRPV3, TRPV1, TRPA1, and TRPC1 that are now discovered to be biomarkers of high-risk SCC and non-melanoma cancers.

The term, “kit” as used herein, means any manufacture (e.g., a package or container) comprising at least one reagent, e.g., a TRPV3 agonist for treatment of SCC (including high-risk forms), non-melanoma cancers (including high-risk forms), and AK. In certain embodiments, the manufacture may be promoted, distributed, or sold as a unit for performing the methods of the present invention.

The term “monoterpenes” as used herein, are a group of naturally occurring organic compounds (like camphor, borneol or methol) derived from two isoprene units. Most of them are fragrant and form major constituents of many plant-derived essential oils. A number of monoterpenes have also been described as agonists of TRPV3 and are set forth in Table 1.

The term “non-melanoma skin cancers” include: basal cell carcinoma, squamous cell carcinoma, angiosarcoma, cutaneous B-cell lymphoma, cutaneous T-cell lymphoma, dermatofibrosarcoma protuberans, merkel cell carcinoma, sebaceous gland carcinoma, and other cancers that spread from other areas of the body to the skin, such as breast cancer and mouth cancers.

The term “prophylactically effective amount” as used herein, means an amount of a drug that, when administered to a subject, will have the intended prophylactic effect, e.g., preventing or delaying the onset (or reoccurrence) of the disease or symptoms, or reducing the likelihood of the onset (or reoccurrence) of the disease or symptoms. The full prophylactic effect does not necessarily occur by administration of one dose and may occur only after administration of a series of doses. Thus, a prophylactically effective amount may be administered in one or more administrations. An “effective amount” of an agent is an amount that produces the desired effect.

The term “receptor” as used herein, means a structure expressed by cells and which recognizes binding molecules (e.g., ligands).

The term “sample” as used herein means, any biological specimen obtained from a subject. Samples include, without limitation, a tissue sample (e.g., tumor tissue) such as a biopsy of a tumor or of an area of skin having or suspected of having SCC (including high-risk forms), non-melanoma cancers (including high-risk forms), or AK, the tissue is typically exposed skin but can also be lips, mouth, esophagus, urinary bladder, prostate, lung, vagina, and cervix. A biopsy of cells of a solid tumor or of skin suspected of having SCC (including high-risk forms), non-melanoma cancers (including high-risk forms), or AK, can be obtained using any technique known in the art. While exposed lesions are typically treated topically, non-exposed lesions can be treated by local administration such as by injection to the lesion which would enable the use of higher concentrations without causing toxicity to the subject, or by systemic administration

The term “significantly higher” as used herein with regard to biomarker expression, means that levels of one or more TRP ion channels (e.g., TRPV3, TRPV1, TRPA1, and TRPC1), or of mRNA encoding the TRP channels, in a subject biopsy show a statistically significant increase over control levels in normal skin without keratinocyte lesions. On the other hand, the term “significantly lower” as used herein, means levels of one or more TRP channels, or of mRNA encoding the TRP channels, in a subject biopsy show e a statistically significant decrease below control levels. Certain embodiments provide transcript levels that were determined to differ (either “significantly higher” or “significantly lower”) between tissues by considering the variability of the qPCR assay. Means from qPCR technical replicates (typically 3-6 per sample) were compared with Student's t tests (two-tailed). Expression is therefore considered to be “significantly higher” or “significantly lower” if the t test indicates that the means differ at the P≦0.05 level.

The term “squamous cell carcinoma” (“SCC”) as used herein, means a cancer of a kind of epithelial cell, the squamous cell that make up the main part of the epidermis of the skin. SCC is one of the major forms of skin cancer. However, squamous cells also occur in the lining of the digestive tract, lungs, and other areas of the body, and SCC occurs as a form of cancer in diverse tissues, including the lips, mouth, esophagus, urinary bladder, prostate, lung, vagina, and cervix, among others. SCC is a histologically distinct form of cancer arising from the uncontrolled multiplication of cells of epithelium, or cells showing particular cytological or tissue architectural characteristics of squamous cell differentiation, such as the presence of keratin, tonofilament bundles, or desmosomes, structures involved in cell-to-cell adhesion. Squamous cell carcinomas are at least twice as frequent in men as in women. They rarely appear before age fifty and are most often seen in individuals in their seventies. The majority of skin cancers in African-Americans are squamous cell carcinomas, usually arising on the sites of preexisting inflammatory skin conditions or burn injuries. SCC is still sometimes referred to as “epidermoid carcinoma” and “squamous cell epithelioma,” though the use of these terms has decreased.

The term “terpene” as used herein means a large and diverse class of organic compounds, produced by a variety of plants, particularly conifers, though also by some insects such as termites or swallowtail butterfly. Some terpenes are major constituents of camphor oil. Several of these, including linalool, pinene, limonene (dipentene), geraniol and borneol are TRPV3 agonists. In particular linalool has been reported to make up 90% of camphor oil in one study, and borneol has been reported to be more effective than camphor as a TRPV3 agonist.⁵⁰

The term “therapeutically effective amount” as used herein means an amount that achieves the intended therapeutic effect of reducing or controlling or eliminating a keratinocyte-derived lesion such as SCC or AK, including precancerous lesions and benign tumors having the morphological characteristics of SCC in a subject. The full therapeutic effect does not necessarily occur by administration of one dose and may occur only after administration of a series of doses. Thus, a therapeutically effective amount may be administered in one or more administrations per day for successive days.

The term “transient receptor potential channel” or “TRP channel” as used herein, mean a group of ion channels located mostly on the plasma membrane of numerous human and animal cell types. There are about 28 TRP channels that share some structural similarity to each other. These are grouped into two broad groups: group 1 includes, TRPC (“C” for canonical), TRPV (“V” for vanilloid), TRPM (“M” for melastatin), TRPN and TRPA. In group 2, there are TRPP (“P” for polycystic) and TRPML (“ML” for mucolipin). Many of these channels mediate a variety of sensations like the sensations of pain, hotness, warmth or coldness, different kinds of tastes, pressure, and vision. In the body, some TRP channels are thought to behave like microscopic thermometers and used in animals to sense hot or cold. Some TRP channels are activated by molecules found in spices like garlic (allicin), chilli pepper (capsaicin), wasabi (allyl isothiocyanate); others are activated by menthol, camphor, peppermint, and cooling agents; yet others are activated by molecules found in cannabis (i.e. THC, CBD and CBN). Some act as sensors of osmotic pressure, volume, stretch, and vibration.

The term “transient receptor potential vanilloid 3” or “TRPV3” protein as used herein, means a nonselective calcium cation channel that is proposed to function in a variety of processes, including temperature sensation and vasoregulation. The TRPV3 channel is widely expressed in the human body, especially in the skin in keratinocytes, but also in the brain. It is a thermosensitive ion channel expressed predominantly in the skin and neural tissues. It is activated by warmth and the monoterpene camphor and has been hypothesized to be involved in skin sensitization.

The term “treating” a disease such as SCC cancer (including high-risk forms), or non-melanoma cancers (including high-risk forms), or AK in a patient as used herein, means taking steps to obtain beneficial or desired results, including clinical results. For purposes of this invention, beneficial or desired clinical results include, but are not limited to alleviation or amelioration of one or more symptoms of the SCC cancer (including high-risk forms), or non-melanoma cancers (including high-risk forms), or AK; diminishing the extent of disease; delaying or slowing disease progression; amelioration and palliation or stabilization of the disease state.

The terms “treat” or “treatment” as used herein, mean both therapeutic treatment and prophylactic or preventative measures, wherein the object is to prevent or slow down (lessen) an undesired physiological change or disorder, such as the development, progression or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already having cancer and those with benign tumors or precancerous lesions.

The term “tumor” as used herein, comprises one or more cancer cells or benign cells or precancerous cells.

The term “2-aminoethoxydiphenyl borate” or “2-APB” as used herein, means a chemical that acts to inhibit IP₃ receptors, Orai channels and TRP channels (although it activates TRPV1, TRPV2, & TRPV3 at higher concentrations) and derivatives thereof. In research it is used to manipulate intracellular release of calcium ions (Ca²⁺) and modify TRP channel activity.

### 2. Treatment of Cancerous and Precancerous Keratinocyte-Derived Lesions with Camphor, Camphor Oil or Constituents or Derivatives Thereof and/or 2-APB

It has been discovered that the herein-described active agents including (a) camphor oil, including camphor white oil from which camphor has been substantially removed, and (b) camphor oil constituents or derivatives thereof, most notably camphor, alpha-pinene and dipentene, as well as (c) terpene agonists of TRPV3 calcium-permeable cation channels such as 2-APB, are effective in treating keratinocyte-derived cancerous or precancerous lesions. These lesions include any precancerous lesions such as AK and both nonaggressive and high risk forms of nonmelanoma cancers including as SCC (herein collectively “the enumerated diseases”).

Experiments showed that human biopsy-derived SCC cells cultured in vitro in a preclinical organotypic model responded to the topical application of the terpene TRPV3 agonist, 2-APB or camphor, by showing reduced cancer cell proliferation which in turn reduced tumor burden and invasiveness. Topical application of camphor white oil in vivo promoted regression of pre-malignant SCC skin tumors, slowed progression of benign tumors to SCC; suppressed SCC invasion; and dramatically attenuated malignant SCC conversion in vivo. Topical camphor-oil treatment was also sufficient to clear apparent tumors in a subset of animals in carcinogenesis models in vivo. Additional in vivo experiments with dipentene and α-pinene, components of camphor white oil also showed striking tumor reductions; both are constituents of camphor oil.

Without wishing to be bound by theory, one hypothesis is that camphor, 2-APB, camphor oil and its constituents and derivatives reduce tumor burden by directly decreasing keratinocyte proliferation, increasing differentiation and/or increasing cell death. Alternatively, these terpenes might modulate the tumor microenvironment in ways that attenuate tumor cell growth (e.g. increasing anti-tumor immunity, extracellular matrix, or vasculature stabilization).

It has also been discovered that high-risk SCC or high-risk non-melanoma skin cancer can be diagnosed by determining in a biopsy of a keratinocyte lesion from a subject if the level of expression of one or more TRP channels, including TRPV3, TRPC1, TRPV1, TRPA1, is either significantly higher or significantly lower compared to the corresponding level in normal skin, either from the same or a different subject.

### 3. Overview

The incidence of human squamous cell carcinomas and non-melanoma skin cancer is at an all-time high (Ratushny et al., 2012). Although the standard of care (e.g., surgery) is adequate for low risk cases, patients with more aggressive tumors show high incidence of recurrence. For example, SCCs that develop in chronic immunosuppression, such as in organ transplant recipients and patients with HIV, are frequently highly aggressive and potentially fatal tumors. Little is known about the pathophysiological mechanisms underlying keratinocyte-derived skin cancers and their relation to normal keratinocyte growth and differentiation. A better understanding of normal keratinocyte maturation is critical for unmasking the pathophysiological changes resulting in tumorigenesis. Environmental factors and genetic alterations that contribute to keratinocyte-derived skin cancers have been identified; however, a better understanding of normal keratinocyte physiology is critical for unmasking pathophysiological changes resulting in tumorigenesis.

Non-melanoma skin cancers are the most common cancers in the United States. Little is known about the pathophysiological mechanisms underlying keratinocyte-derived skin cancers and their relation to normal keratinocyte growth and differentiation. The human epidermis is a multilayered stratified epithelium whose differentiation program is triggered in part by increased Ca²⁺ levels. The role of Ca²⁺ in epidermal physiology, including the molecular basis for differentiation, is not fully understood. It is known that Ca²⁺ triggers the commitment switch from keratinocyte proliferation to differentiation. As non-melanoma skin cancers typically exhibit perturbed patterns or dramatic loss of keratinocyte differentiation, it is important to determine the role for the calcium ion channels TRPV3, TRPV1, TRPA1, and TRPC1 in human keratinocyte maturation and its potential as a target for skin cancer therapy.

### Squamous Cell Carcinoma

SCC is a cancer of a kind of epithelial cell, the squamous cell that makes up the main part of the epidermis of the skin. SCC is one of the major forms of skin cancer. However, squamous cells also occur in the lining of the digestive tract, lungs, and other areas of the body, and SCC occurs as a form of cancer in diverse tissues, including the lips, oral mucosa, nasopharynx, esophagus, urinary bladder, prostate, lung, vagina, and cervix, among others. SCC is a histologically distinct form of cancer arising from the uncontrolled multiplication of cells of epithelium, or cells showing particular cytological or tissue architectural characteristics of squamous cell differentiation, such as the presence of keratin, tonofilament bundles, or desmosomes, structures involved in cell-to-cell adhesion. Squamous cell carcinomas are at least twice as frequent in men as in women. They rarely appear before age 50 and are most often seen in individuals in their 70s. The majority of skin cancers in African-Americans are squamous cell carcinomas, usually arising on the sites of preexisting inflammatory skin conditions or burn injuries. SCC is still sometimes referred to as “epidermoid carcinoma” and “squamous cell epithelioma”, though the use of these terms has decreased.

Squamous cell carcinoma is the second-most common cancer of the skin (after basal cell carcinoma) and it is more common than melanoma. World-wide, it is the most common cancer that has the potential to metastasize. It usually occurs in areas exposed to the sun. Sunlight exposure and immunosuppression are risk factors for SCC of the skin, with chronic sun exposure being the strongest environmental risk factor. Other risk factors include fair skin, age, male gender, history of skin cancer, and smoking. There is a risk of metastasis, often spreading to the lymph nodes, starting more than 10 years after diagnosable appearance of SCC. The risk of SCC metastasis is low, though it is much higher than with basal cell carcinoma. It is important to note that SCC of the lip and ears has significantly higher rates of local recurrence and distant metastasis (20-50%). SCC of the skin in individuals on immunotherapy or suffering from lymphoproliferative disorders (i.e. leukemia) also tend to be much more aggressive, regardless of their location. SCCs represent about 20% of the non-melanoma skin cancers, but due to their more obvious nature and growth rates, they represent 90% of all head and neck cancers that are initially presented.

Squamous cell carcinoma is generally treated by surgical excision or Mohs surgery after biopsy. While it is relatively easy to identify SCC, it is more challenging to diagnose a high-risk form of SCC. Morphological factors that indicate a high-risk form of SCC include the depth of the tumor (at >2 cm), poorly differentiated cells, ulceration, the location of the area involved (e.g., ears, face, scalp are at higher risk), and intravascular invasion. Other high-risk factors for SCC include the immune status of the subject (immune compromised subjects have a higher risk of SCC), and whether or not the abnormal cells involve the nervous system. Immunocompromised patients (e.g., organ transplant patients, “OTR”) have an increased risk of developing aggressive, high-risk SCC and high-risk non-melanoma cancers. Immunocompromised patients (e.g., organ transplant patients, “OTR”) have an increased risk of developing aggressive, high-risk SCC and high-risk non-melanoma cancers, and biopsies from such individuals should routinely be tested to determining if there is a significantly higher or significantly lower level of mRNA encoding one or more of the following biomarkers of high-risk SCC/non-melanoma cancer: TRPV3, TRPC1, TRPV1, and TRPA1 to help determine the course of treatment. If an OTR subject does not have abnormal biomarker mRNA levels, the subject may respond to non-aggressive surgical removal of the SCC combined with TRPV3, TRPV1, TRPA1, and TRPC1 agonist therapy, and not require the more aggressive surgery.

High-risk forms of SCC and non-melanoma cancers are aggressive and therefore require more aggressive surgery (including taking wider margins, cutting deeper, and removing lymph nodes) as well as adjuvant therapy, than is needed if the SCC is not high-risk. Until now, there were no biomarkers for distinguishing high-risk from non-high-risk SCC and non-melanoma cancer. It has now been discovered that either a significantly higher or significantly lower level of mRNA encoding one or more of the following: TRPV3, TRPV1, TRPA1, and TRPC1 is a biomarker for high-risk SCC and high-risk non-melanomas. Therefore, SCC and non-melanoma biopsies, especially OTR or other immunocompromised patients, can now be tested to determine whether they are high-risk by determining if the expression of one or more of these biomarkers is significantly higher or significantly lower compared to a corresponding level in a normal subject. If the SCC or the non-melanoma is a high-risk cancer, it warrants aggressive treatment: aggressive surgery and adjuvant therapy, including the newly described therapy of administering one or more active agents of the invention to treat the cancer. Even if the cancer is not high-risk, treatment should include administration of therapeutically effective amounts of one or more active agents as described herein, preferably before and after removal of the lesion and/or other treatment of the lesion. If the over or underexpression of the one or more of the biomarkers is detected in a biopsy from a subject, it is recommended that treatment, be started as soon as possible, even before the biopsy results are received. In the case of high-risk SCC/high-risk non-melanoma, treatment should be continued for a period of time after surgery to assure that all cancerous or precancerous cells are killed. It is preferred that all SCC and non-melanoma subjects, both high-risk and non-aggressive cancers, receive topical application of one or more active agents to the site from which the SCC or non-melanoma skin cancer was removed for an extended period of time, possibly indefinitely, to prevent abnormal keratinocyte proliferation and/or cancer recurrence.

Non-surgical options for the treatment of cutaneous SCC include topical chemotherapy, topical immune response modifiers, photodynamic therapy (PDT), radiotherapy, and systemic chemotherapy. Radiation therapy is a primary treatment option for patients in whom surgery is not feasible and is an adjuvant therapy for those with metastatic or high-risk cutaneous SCC. At this time, systemic chemotherapy is used exclusively for patients with metastatic disease. Mohs surgery, also known as chemosurgery, enables the surgeon to obtain complete margin control during removal of a skin and it allows for the removal of a skin cancer with very narrow surgical margin and a high cure rate.

### Actinic Keratosis

Actinic keratosis (also called “solar keratosis” and “senile keratosis,” herein “AK”) is a premalignant condition of thick, scaly, or crusty patches of skin. It is more common in fair-skinned people and it is associated with those who are frequently exposed to the sun, as it is usually accompanied by solar damage. AKs are pre-cancerous lesions, as some progress to squamous cell carcinoma, so treatment is recommended. Untreated lesions have up to 20% risk of progression to squamous cell carcinoma. People who take immunosuppressive drugs, such as organ transplant patients, are 250 times more likely to develop AK that may lead to skin cancer.

Medicated creams and solutions are typically used topically to treat actinic keratosis. The use of topical therapy, such as Imiquimod cream and PDT is generally limited to premalignant (i.e., AKs) and in situ lesions. 5-fluorouracil (5-FU) ointment or liquid in concentrations from 0.5 to 5 percent has FDA approval and is the most widely used topical treatment for AK as it is effective against not only the surface lesions but also the subclinical ones. Rubbed gently onto the lesions once or twice a day for two to four weeks, it produces cure rates of up to 93 percent. Imiquimod 5% cream, also FDA-approved, works by stimulating the immune system to produce interferon, a chemical that destroys cancerous and precancerous cells. It is rubbed gently on the lesion twice a week for four to sixteen weeks. Diclofenac is a non-steroidal anti-inflammatory drug used in combination with hyaluronic acid. The resulting gel is applied twice a day for two to three months to prevent an inflammatory response, so this topical is well-tolerated. The hyaluronic acid delays uptake of the diclofenac, leading to higher concentrations in the skin. The gel, used in 0.015% or 0.05% concentrations depending on the AK site, is the first topical therapy to effectively treat AKs in just two or three days. Certain embodiments are directed to pharmaceutical formulations for topical applications of the above listed formulations that further include one or more of the active agents as described herein. Cryosurgery is the most commonly used treatment method when a limited number of lesions exist. Other treatment includes laser surgery, and photodynamic therapy.

### TRPV3 Agonists, Camphor Oil and Derivatives and Constituents Thereof

Several calcium-permeable nonselective cation channels of the transient receptor potential (TRP) family have been implicated in normal keratinocyte function. Keratinocytes express TRPV3 and TRPV4, and mutations in these receptors cause epidermal abnormalities such as barrier defects and hyperkeratosis (Cheng et al, 2010; Chung et al, 2004b; Kida et al, 2012; Lin et al, 2012). In mice, TRPV3 regulates terminal keratinocyte differentiation via activation of EGFR signaling (Cheng et al, 2010). EGFR signaling also potentiates TRPV3 activity, illustrating that positive feedback links these receptors (Cheng et al, 2010). EGFR overexpression is a characteristic feature of SCC, and activation of EGFR correlates with poor prognosis in SCC (Hardisson, 2003). A number of EGFR inhibitors are FDA-approved for SCC treatment but their efficacies are inconsistent and some are associated with severe skin toxicity (Bauman et al, 2012).

TRPV3 receptors are mechanistically distinct from voltage-gated calcium channels. Voltage-gated calcium channels respond to membrane depolarization and open to permit an influx of calcium from the extracellular medium that result in an increase in intracellular calcium levels or concentrations. TRPV3 proteins are thermo sensitive channels expressed in skin cells.⁸⁷ (and dorsal root ganglion, trigeminal ganglion, spinal cord and brain^{88, 89} In keratinocyte cell lines, stimulation of TRPV3 leads to release of inflammatory mediators including interleukin-1. U.S. Ser. No. 13/348,272 describes a method for treating a long list of diseases including AK by administering some newly discovered TRPV3 antagonists that have a different ring structure than either camphor or 2-APB.⁹⁰ U.S. application Ser. No. 13/175,366 describes only TRPV3 antagonists and mentions agonists only generally in the context of screening assays. (91. Moran et al., Compounds for Modulating TRPV3 Function,) Because of the role TRPV3 plays in keratinocyte function, it was decided to test certain TRPV3 agonists to see if they affect precancerous and cancerous keratinocyte-derived lesions. Initial experiments began with camphor and 2-APB, then moved to camphor white oil and various constituents and derivatives thereof. Studies were extended to include camphor oil and other terpenes identified in camphor oil, which can be formulated at higher doses for topical applications.

Different terpenes have been shown to activate, inactivate or modulate ion channels. A number of terpenes have been described as agonists or antagonists of different members of the transient receptor potential (TRP) channel family (Mckemy et al., 2002; Peier et al., 2002a, b; Behrendt et al., 2004; Moqrich et al., 2005; Xu et al., 2005, 2006; Macpherson et al., 2006). Table 1 below shows certain terpene TRPV3 agonists screened from 33 different terpenes. (Vogt-Eisele et al Br J Pharmacol 2007 151:530-540; PMID17420775). Borneol has been reported to be more effective than camphor as a TRPV3 agonist.

TABLE 1
Terpenoid agonists of TRPV3

| | |
|---|---|
| Camphor | (+)-Borneol |
| (−)-Isopinocampheol | (−)-Fenchone |
| (−)-Trans-pinocarveol | Isoborneol |
| (+)-Camphorquinone | (−)-alpha-Thujone |
| Alpha-pinene oxide | 1,8-Cineole |
| (−)-alpha-Pinene | Isobornyl acetate |
| 6-tert-butyl-m-cresol | Carvacrol |
| Thymol | p-xylenol |
| Kreosol | Propofol |
| p-cymene | Carvacrol methylether |
| Dihydrocarveol | (−)-Carveol |
| (−)-Isopulegol | (−)-Menthol |
| (−)-Carvone | (+)-Dihydrocarvone |
| (−)-Menthone | (+)-Limonene [dipentene] |
| Terpineol | (+)-Linalool |
| Geraniol | 1-Isopropyl-4-methyl-bicyclo [3.1.0] |
| | hexan-4-ol |
| (−)alpha-Bisabolol | Mugetanol |

Camphor is an organic compound of the isoprenoid family with the chemical formula C₁₀H₁₆O that belongs to the group of bicyclic monoterpenes. Camphor is readily absorbed through the skin and produces substance feeling of warmth, and acts as a slight local anesthetic and anti-itch substance. Camphor is a distinct terpene constituent of many C. camphora extracts and it is an agonist of TRPV3 cation channels that govern keratinocyte differentiation pathway. However it is noted that camphor was found at trace levels or lower in the camphor white oil that was used in the in vivo experiments described here.

Camphor oil is an essential oil, which is derived from the camphor laurel tree (Cinnamomum camphora), is marketed as a fragrance and herbal remedy with antifungal, antiseptic and medicinal properties, including circulatory stimulation, increased metabolism and improved digestion. Camphor oil and its terpene constituents are widely found in natural products (see Table 1) and have been used for centuries for medicinal purposes. In traditional Eastern medicine, C. camphora extracts are used for congestion, bronchitis and cardiac ailments. Camphor oil's terpene constituents are commonly used in commercial manufacturing, culinary and medicinal applications. For example, eucalyptol often is used in mouthwash and cough suppressants. Dipentene, also known as limonene, is prominent in citrus oils used as flavorings in foods and beverages, and is a solvent in household cleaning products. α-pinene confers pine scent in household products. γ-Terpinene is frequently used in topical cosmetics. As alternative remedies, essential oils are most often administered topically or via inhalation, and they can be toxic when ingested Like other essential oils, camphor oil exhibits oral toxicity and skin irritation at high concentrations. The irritant properties of camphor oil are notable because they demonstrate that camphor oil penetrates the skin barrier and exerts local effects on cells in the skin.

Camphor oil can be a natural extract or a synthetic mixture of components (e.g., CAS number 8008-51-3). Monoterpenes other than camphor are major constituents of camphor oil. Linalool has been reported to make up 90% of camphor oil in one study. Terpenes (like camphor, borneol or methol) comprise a group of naturally occurring organic compounds derived from isoprene units. Most of them are fragrant and form major constituents of many plant-derived essential oils. While most are commonly used as antimicrobial agents, terpenes have a wide range of applications in pharmaceutical, medical and cosmetic fields. These uses range from anesthetic and analgesic (Galeotti et al., 2001, 2002; Xu et al., 2005) to anti-inflammatory (Santos and Rao, 2001) and antipruritic applications (Umezu et al., 2001; Anand, 2003).

The in vivo experiments described here were done primarily with camphor white oil from which camphor had been removed by distillation. Some in vivo experiments were done with camphor. GC-MS analysis performed on two independent formulations of naturally derived camphor white oil, showed that it was made primarily of terpenes. See Table 2. Terpenes are small molecules composed of five-carbon isoprene units. There was substantial variability in components between formulations. Although camphor is abundant in C. camphora leaf extracts, there were only trace amounts (<0.1%) detected in both lots of the camphor white oil tested.

TABLE 2
Components/Constituents of Natural Camphor White Oil

| | | | % in | % in | |
|---|---|---|---|---|---|
| CAS# | Compound | Class | Lot 1 | Lot 2 | Library/ID |
| 470-82-6 | Eucalyptol | terpenoid | 29.37 | 31.73 | C:\Database\wiley7n.l,8- |
| | | | | | Cineole $$ 2-Oxabicyclo[2.2.2] |
| | | | | | octane, 1,3,3-trimethyl- |
| 138-86-3 | Dipentene | cyclic terpene | 19.14 | 24.46 | C:\Database\wiley7n.l dl- |
| | | solvent | | | Limonene |
| 80-56-8 | α-Pinene | bicyclic | 11.6 | 1.77 | C:\Database\wiley7n.l.ALPHA.- |
| | | terpene | | | PINENE, (−)- |
| 3387-41-5 | Sabinene | bicyclic | 4.83 | 4.93 | C:\Database\wiley7n.l Sabinene |
| | | terpene | | | |
| 79-92-5 | Camphene | bicyclic | 4.24 | 0.6 | C:\Database\wiley7n.l.Camphene |
| | | terpene | | | |
| 99-83-2 | α- | cyclic terpenes | 0.95 | 1.93 | C:\Database\wiley7n.l 1- |
| | Phellandrene | | | | Phellandrene |
| 934-80-5 | 4-ETHYL-O- | | 13.05 | | C:\Database\wiley7n.lBenzene, |
| | XYLENE | | | | 4-ethyl-1,2-dimethyl- |
| 99-85-4 | γ-Terpinene | terpene | 6.7 | | C:\Database\wiley7n.l.gamma.- |
| | | | | | Terpinene |
| 18172- | (−)-β-Pinene | bicyclic | 4 | | C:\Database\wiley7n.l |
| 67-3 | | terpene | | | Bicyclo[3.1.1]heptane, 6,6- |
| | | | | | dimethyl-2-methylene-, (1S)- |
| 1529-99-3 | Alpha | | 2.73 | | C:\Database\wiley7n.l.ALPHA. |
| | Phellnadrene | | | | PHELLANDRENE |
| 99-86-5 | α-Terpinene | terpene | 1.41 | | C:\Database\wiley7n.l.alpha.- |
| | | | | | Terpinene |
| 586-62-9 | Terpinolene | terpene | 1.26 | | C:\Database\wiley7n.l.ALPHA.- |
| | | | | | TERPINOLENE |
| 13466- | 3-Carene | bicyclic | 0.54 | | C:\Database\wiley7n.l.DELTA. |
| 78-9 | | terpene | | | 3 CARENE |
| 76-22-2 | (±)-Camphor | bicyclic | 0.17 | | C:\Database\wiley7n.l Camphor |
| | | terpene | | | |
| 25155- | cymene | alkylbenzene | | 18.77 | C:\Database\wiley7n.l Benzene, |
| 15-1 | | related to a | | | methyl(1-methylethyl)-(CAS) |
| | | terpene | | | |
| 127-91-3 | (−)-β-Pinene | bicyclic | | 7.86 | C:\Database\wiley7n.l 2- |
| | | terpene | | | .BETA.-PINENE |
| 554-61-0 | 2-Carene | bicyclic | | 5.51 | C:\Database\wiley7n.l |
| | | terpene | | | Bicyclo[4.1.0]hept-2-ene, 3,7,7- |
| | | | | | trimethyl- |
| 116-02-9 | 3,3,5-trimethyl- | | | 0.71 | C:\DATABASE\NIST98.L |
| | Cyclohexanol | | | | Cyclohexanol, 3,3,5-trimethyl- |
| 500-02-7 | 4-isopropyl-2- | | | 0.63 | C:\Database\wiley7n.l 2- |
| | cyclohexenone | | | | Cyclohexen-1-one, 4-(1- |
| | | | | | methylethyl)- |
| 632-16-6 | 2,3-dimethyl | | | 0.57 | C:\Database\wiley7n.l |
| | thiophene | | | | Thiophene, 2,3-dimethyl-2,3- |
| | | | | | Dimethylthiophene |
| 142-30-3 | 2,5-Dimethyl- | | | 0.36 | C:\Database\wiley7n.l 3- |
| | 3-hexyne-2,5- | | | | Hexyne-2,5-diol, 2,5-dimethyl- |
| | diol | | | | |
| 000000- | 2-Hydroxyl-2-methyl-5- | | | 0.18 | C:\Database\wiley7n.l 2- |
| 00-0 | isopropenyl-1-methylenecyclo- | | | | Hydroxyl-2-methyl-5- |
| | pentane | | | | isopropenyl-1-methylenecyclo- |
| | | | | | pentane |

Identified by GC-MS analysis. *Lot #1 was used for all preliminary experiments.

As noted above, other components of camphor oil include 1,8-cineole, Furfural, Linalool, Bornyl acetate, Terpinen-4-ol, Caryophyllene, Borneol, Piperitone, Geraniol, Safrole, Cinnamaldehyde, Methyl cinnamate, Eugenol.

TABLE 3
TRP channel agonists and antagonists.
Each compound's target(s) and the anticipated action are listed. The concentration range
and/or the half maximum dose (EC₅₀ or IC₅₀) are given for each respective target.

| | Targeted TRP | | | |
|---|---|---|---|---|
| Compound | channels | Action | Effective concentration | Reference |
| 2-APB | TRPV3 | Activation | EC50 = 28 μM | (Chung et al, |
| | | | Range = 3.2-320 μM | 2004) |
| | TRPC1 | Activation | Range = 5-10 μM | (Vanden Abeele |
| | | Inhibition | Range = 50-100 μM | et al, 2003) |
| | ORAI1 | Inhibition | Range = 5-50 μM | (Soboloff et al, |
| | | | | 2006) |
| Camphor | TRPV3 | Activation | Range = 2-10 mM | (Moqrich et al, |
| | | | | 2005) |
| | TRPV1 | Activation | Range = 1-10 mM | (Xu et al, 2005) |
| | TRPA1 | Inhibition | IC50 = 68 μM | (Macpherson et |
| | | | Range = 10 μM-10 mM | al, 2006) |

### 4. Summary of Experimental Results and Embodiments of the Invention

In summary, it has been discovered that camphor oil and camphor oil constituents including camphor, dipentene and alpha-pinene, as well as agonists of TRPV3 such as 2-APB, have therapeutic use in treating any cancerous or precancerous keratinocyte-derived lesions including SCC (including high-risk forms), non-melanoma skin cancers (including high-risk forms), and AK. Support comes in part from experiments described herein where human biopsy-derived SCC cells cultured in vitro in a preclinical organotypic model, were found to express functional TRPV3. These SCC cells responded to the terpene TRPV3 agonists 2-APB and camphor that were topically applied individually with reduced cancer cell proliferation and reduced tumor burden and invasion.

Other in vivo experiments were done with topical camphor white oil treatment that also reduced SCC tumor burden, promoted regression of pre-malignant skin tumors and slowed progression of benign tumors to SCC. Dose-dependent effects of 2-APB and camphor oil were also observed on normal keratinocyte behavior. The following is a summary of results of experiments described in the Examples of this application. Identification of epidermal TRP channels as key regulators of the commitment switch from proliferation to differentiation in human epidermal keratinocytes;
TRP-channel agonists induce fate switch from proliferation to differentiation in human keratinocytes;
Treatment for ≧16 h with 2-APB or camphor arrested human normal keratinocyte proliferation and promoted differentiation;
TRP-channel gene expression and protein function was upregulated in normal human epidermal keratinocytes upon differentiation;
TRP-channel gene expression (TRPV3, TRPV1, tRPA1, and TRPC1) was dysregulated in human high-risk SCC biopsies compared with normal skin. Quantitative expression analysis demonstrated that TRPV1, TRPV3, TRPC1 transcript levels were higher in keratinocytes cultured in differentiation compared with proliferative conditions;
Calcium imaging revealed that the responses of normal human epidermal keratinocytes to acute application of the camphor or 2-APB increased upon differentiation;
24-hour incubation with 2-APB in low-calcium growth conditions boosted the expression of differentiation genes;
Both 50 μM 2-APB and 4-8 mM camphor caused cell-cycle arrest, a hallmark for the commitment switch from proliferation to differentiation;
Stimulating TRP-channel activity is sufficient to induce early differentiation which enhanced TRP-mediated calcium signaling, caused either by raising extracellular calcium or by exposure to TRP agonists, promotes differentiation in normal human epidermal keratinocytes;
2-APB reduced human SCC tumor growth and dermal invasion in vitro;
Topical camphor white oil treatment promoted regression of pre-malignant SCC skin tumors in vivo. Dipentene and α-pinene also showed striking tumor reductions compared with vehicle, although less dramatic than camphor white oil;
Topical camphor white oil treatment slowed progression of benign tumors to SCC in vivo;
Topical camphor white oil treatment suppressed SCC proliferation and invasion in vivo;
Topical camphor white oil treatment dramatically attenuated malignant SCC conversion in vivo;
Topical camphor white oil treatment was sufficient to completely clear tumors in a subset of animals in carcinogenesis models in vivo; and
Treatment with camphor upregulated levels of a keratinocyte terminal differentiation marker in vivo.

### 5. Embodiments of Methods of Treatment and Diagnostic Methods

Embodiments of the invention provide methods of diagnosing, and treating subjects having keratinocyte-derived lesions as described including SCC (including high-risk forms), non-melanoma skin cancers (including high-risk forms), and AK. In certain embodiments subjects at risk of developing non-melanoma skin cancer such as SCC, or AK, including immunocompromised subjects, including transplant recipients, are treated with topical therapy.

Certain embodiments of the invention are directed to methods of treating a keratinocyte lesion, including SCC and non-melanoma cancers and AK by administering a therapeutically effective amount of an active agent such as camphor oil, including white camphor oil; a constituent of camphor oil; a derivative of camphor oil or a constituent thereof; or a terpene TRPV3 agonist as described herein. In certain embodiments camphor, 2-APB, dipentene or alpha-pinene is administered. Exposed lesions are preferably treated topically. Other routes of administration and therapeutic dose ranges are discussed below. In some embodiments the agent slows progression of a benign or precancerous lesion to a cancerous lesion or promotes regression of a pre-cancerous or cancerous lesion.

Certain other embodiments are directed to pharmaceutical compositions comprising therapeutically effective amounts of an active agent selected from the group consisting of terpenoid TRPV3 agonists, camphor oil, a constituent of camphor oil, or a camphor oil derivative, specifically also camphor, 2-APB, dipentene and alpha-pinene and derivatives of all of these agents.

An embodiment is directed to a method for diagnosing high risk SCC or high risk non-melanoma cancer by determining that a biological sample from a biopsy of a lesion has either significantly elevated or significantly reduced levels of one or more TRP ion channels (TRPV3, TRPV1, TRPA1, and TRPC1) compared to levels in normal skin.

### 6. Pharmaceutical Compositions or Formulations and Their Administration

Embodiments are directed to pharmaceutical compositions that contain one or more active agents, preferably camphor oil (including white camphor oil), terpene TRPV3 agonists such as 2-APB, camphor oil constituents such as camphor, limonene (dipentene) and α-pinene, and camphor oil derivatives, or combinations thereof for treatment of pre-cancerous and cancerous keratinocyte-derived lesions, e.g., SCC (including high-risk forms), non-melanoma skin cancers (including high-risk forms), and AK. Other embodiments are directed to sunscreens containing one or more active agents. These pharmaceutical compositions and kits comprising them may be formulated as described below and are typically in topical formulations including creams, ointments, paste, powers, lotions, and gels. One embodiment is an ointment that is topically applied. In some formulations the ointment is hydrophobic enough to keep the camphor in the ointment but also hydrophilic enough that it would not dry the skin. The contemplated pharmaceutical compositions include the active agents described herein in a therapeutically effective amount sufficient to treat one or more of the enumerated diseases: SCC, (including high-risk forms) non-melanoma skin cancers (including high-risk forms), or AK in a subject or prevent reoccurrence. The doses of active agents for formulations to treat the enumerated diseases are discussed below.

The amount of active agent will vary depending on many factors, including the severity of the disease, the size of the lesion, the location of the lesion, the age, sex and immune status of the subject, the pharmacokinetics of the agent, frequency of administration and the efficacy. As described above, in preferred embodiments, therapeutically effective amounts of camphor and camphor oil range from 0.0608-99.5% (wt/vol or vol/vol), preferably in the range of 10-50%, and 2-APB ranges from about 0.00005-05%.

Various factors known to those skilled in the art affect the actual therapeutic amounts used in vivo, especially in humans. In the in vitro and in vivo experiments described herein, there was no barrier to penetration of the active agent, which was applied topically. In vivo mouse studies showed that 10-30% camphor-oil had therapeutic utility in treating SCC. Higher or lower doses may also be effective as is discussed herein in vivo. Penetration of the active agents to the targeted SCC, non-melanoma cancers, or AK cells can be optimized by adjusting the dose, but also by formulating the active agents in ways that enhance uptake, for example by using skin-penetration agents in the formulations. The active agents of the invention should be applied to the lesion in such a way as to treat the margins of the tumor or affected area in addition to the bulk area of the lesion. Some normal cells will thus be contacted with active agent in this process.

In an embodiment of the invention, the active agents are applied, preferably multiple times, to the affected area having the lesions either for a period of time before the lesion is surgically removed, or for a period of time after is the lesion surgically removed, or more preferably before and after surgery. Application of the active agent before surgery will reduce tumor burden and application after surgery will kill any stray precancerous or cancer cells left behind and reduce the risk of a recurrence. The present therapies can be used in conjunction with other therapies that are effective in treating an enumerated disease. Preferably, the subject is human.

High-risk SCCs up until now were determined by the clinician's general impression based on factors described above. SCCs arising in the head and neck area are typically at higher risk for local invasion and metastasis compared to SCCs arising in the trunk and extremities. Locally advanced, aggressive, recurrent or metastatic SCC is much more difficult to treat. SCCs that arise in the head and neck area, including primary lip, oral cavity, nasal cavity, paranasal sinus, pharyngeal and laryngeal tissues exhibit a local recurrence in 50% of patients and carry an overall survival of only 6-9 months. However, diagnostic methods have now been discovered for high risk SCC and high risk non-melanoma cancers by determining if a biopsy of one or more lesions shows significantly higher or significantly lower expression of one or more of the biomarkers TRPV3, TRPV1, TRPA1, and TRPC1 than normal skin, if so the SCC or non-melanoma cancer is a high-risk. By making a definitive diagnosis the appropriate treatment can be given and over-aggressive treatment of non high-risk cancers can be avoided. For aggressive high risk cancers Mohs surgery followed by chemotherapy and radiation are appropriate. In certain embodiments the biopsy is assayed for mRNA encoding one or more of the TRP channels (TRPV3, TRPC1, TRPV1, and TRPA1) and is compared to the mRNA level in a control sample of normal tissue. In some embodiments the control biopsy can come from a matched biopsy of normal tissue from the diagnosed patient or from another a sample of normal skin from another normal control subject. In a preferred embodiment, if the level of mRNA encoding one or more of the TRP channels (TRPV3, TRPC1, TRPV1, and TRPA1) in the patient biopsy of the affected area is either significantly higher or significantly lower than the level in the control biopsy, then the diagnosis is made that the patient has a high-risk form of SCC or non-melanoma.

The following are commercial camphor products known in the art that can be screened using routine methods known in the art to determine their use in embodiments of the present invention.

Balmosa Cream (camphor 4%, menthol 2%, methyl salicylate 4%, capsicum oleoresin 0.035%) (Pharmax Healthcare)

### Boots Vapour Rub (Boots)

Earex (almond oil 33.33%, arachis oil 33.33%, camphor oil 33.33%) Seton Healthcare)
Mentholatum Vapour Rub (camphor 9%, menthol 1.35%, methyl salicylate 0.33%) (Mentholatum)
Nasciodine (iodine 1.26%, menthol 0.59%, methyl salicylate 3.87%, turpentine oil 3.87%, camphor 3.87%)
Nicobrevin (methyl valerate 100 mg, quinine 15 mg, camphor 10 mg, eucalyptus oil 10 mg) (Intercare Products)
PR Heat Spray (camphor 0.62%, methyl salicylate 1.24%, ethyl nicotinate 1.1%) (Crookes Healthcare)
Radian-B (liniment and spray: menthol 1.4%, camphor 0.6%, ammonium salicylate 1%, salicylic acid 0.54%. rub: menthol 2.54%, camphor 1.43%, methyl salicylate 0.42%, capsicin 0.042%. cream: camphor 1.43%, menthol 2.54%, methyl salicylate 0.42%, oleoresin capsicum 0.005%)Roche Consumer Health)
Tixylix inhalant (camphor 60 mg, menthol 25 mg, turpentine oil 50 mg, eucalyptus oil 20 mg) (Intercare Products)
Vicks Inhaler (camphor 41.54%, menthol 41.54%, siberian pine needle oil 4.65%) %) (Procter and Gamble)
Vicks Sinex (oxymetazoline 0.05%, menthol 0.025%, camphor 0.015%, eucalyptus oil 0.0075%) %) (Procter and Gamble) and
Vicks Vaporub (menthol 2.82%, camphor 5.46%, eucalyptus oil 1.35%, turpentine oil 4.71%) (Procter and Gamble).

The following are non-proprietary preparations known in the art:

Camphor Linctus compound (APF): Camphor spirit compound 1 ml, glycerol 1.5 ml, tolu syrup to 5 ml.
Camphor Liniment (BP 1973): Camphor 20% wt/wt in arachis oil (AKA Camph. Lin; Camphorated oil).
Camphor Spirit (USP): Camphor 10 g, alcohol to 100 ml.
Concentrated Camphor Water (BP) Camphor 4 g, alcohol (90%) 60 ml, water to 100 ml.

In other embodiments one or more active agents, preferably camphor oil, camphor, dipentene, alpha-pinene and/or 2-APB are formulated in sunscreens, (also commonly known as sun block, sun tan lotion, sun screen, sunburn cream or block out) for topical application as a lotion, spray, gel or other product that absorbs or reflects some of the sun's ultraviolet (UV) radiation on skin exposed to sunlight and thus helps protect against sunburn. Depending on the mode of action, sunscreens can be classified into physical sunscreens (i.e., those that reflect the sunlight) or chemical sunscreens (i.e., those that absorb the UV light). Sunscreens contain one or more of the following ingredients: (i) organic chemical compounds that absorb ultraviolet light; (ii) inorganic particulates that reflect, scatter, and absorb UV light (such as titanium dioxide, zinc oxide, or a combination of both); and (iii) organic particulates that mostly absorb light like organic chemical compounds, but contain multiple chromophores, may reflect and scatter a fraction of light like inorganic particulates, and behave differently in formulations than organic chemical compounds.

A wide variety of sun screening agents are described in U.S. Pat. No. 5,087,445, to Haffey et al. U.S. Pat. No. 5,073,372, to Turner et al., U.S. Pat. No. 5,073,371, to Turner et al. and Segarin, et al., at Chapter VIII, pages 189 et seq., of Cosmetics Science and Technology all of which are incorporated herein by reference in their entirety. Preferred among those sunscreens which are useful in the composition of the instant invention are those selected from the group consisting of 2-ethylhexyl p-methoxycinnamate, octyl methoxycinnamate, 1-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide, zinc oxide, silica, iron oxide, and mixtures thereof. Still other useful sunscreens are those disclosed in U.S. Pat. No. 4,937,370, to Sabatelli and U.S. Pat. No. 4,999,186, to Sabatelli et al. The sun screening agents disclosed therein have, in a single molecule, two distinct chromophore moieties which exhibit different ultra-violet radiation absorption spectra. One of the chromophore moieties absorbs predominantly in the UVB radiation range and the other absorbs strongly in the UVA radiation range. These sun screening agents provide higher efficacy, broader UV absorption, lower skin penetration and longer lasting efficacy relative to conventional sunscreens. Especially preferred examples of these sunscreens include those selected from the group consisting of 4-N,N-(2-ethylhexyl)methylanminobenzoic acid ester of 2,4-hydroxybenzophenone, 4-N,N-(2-ethylhexyl)methylaminobenzoic acid ester with 4-hydroxydibenzoylmethane, 4-N,N-(2-ethylhexyl)methylaminobenzoic acid ester of 2-hydroxy-4-(2-hydroxyethoxy)benzophenone, 4-N,N-(2-ethylhexyl)-methylaminobenzoic acid ester of 4-(2-hydroxyethoxy)dibenzoylmethane, and mixtures thereof. Generally, the sunscreens can comprise from about 0.5 percent to about 20 percent of the compositions useful herein. Exact amounts will vary depending upon the sunscreen formulation chosen, the particular active agent, and the desired Sun Protection Factor (SPF). SPF is a commonly used measure of photoprotection of a sunscreen against erythema. See Federal Register, Vol. 43, No. 166, pp. 38206-38269, Aug. 25, 1978.

Medical organizations such as the American Cancer Society recommend the use of sunscreen because it aids in the prevention of developing squamous cell carcinomas and basal-cell carcinomas. However, the use of sunscreens is controversial for various reasons. Many sunscreens do not block UVA radiation, which does not cause sunburn but can increase the rate of melanoma, another kind of skin cancer, and photodermatitis, so people using sunscreens may be exposed to high UVA levels without realizing it. The use of broad-spectrum (UVA/UVB) sunscreens can address this concern.

The following are the FDA allowable active ingredients in sunscreens that can be used in sunscreen formulations comprising the active agents:

| | | Maximum | Permitted in | Results of |
|---|---|---|---|---|
| UV-filter | Other names | concentration | these | safety |
| p-Aminobenzoic | PABA | 15% (EC- | USA, AUS | Protects |
| acid | | banned from | | against |
| | | sale to | | skin |
| | | consumers | | tumors in |
| | | from 8 Oct. | | mice. |
| | | 2009) | | Shown to |
| | | | | increase |
| | | | | DNA |
| | | | | defects |
| Padimate O | OD-PABA, | 8% | EC, USA, | Not tested |
| | octyldimethyl-PABA, | (EC, USA, AUS) | AUS, JP | |
| | σ-PABA | 10% (JP) | | |
| Phenylbenzimidazole | Ensulizole, Eusolex | 4% (US, AUS) | EC, USA, | Genotoxic |
| sulfonic acid | 232, PBSA, Parsol HS | 8% (EC) | AUS, JP | in |
| | | 3% (JP) | | bacteri |
| Cinoxate | 2-Ethoxyethyl p- | 3% (US) 6% | USA, AUS | Not tested |
| Dioxybenzone | Benzophenone-8 | 3% | USA, AUS | Not tested |
| Oxybenzone | Benzophenone-3, | 6% (US) 10% | EC, USA, | Not tested |
| | Eusolex 4360, Escalol | (AUS, EU) 5% | AUS, JP | |
| Homosalate | Homomethyl salicylate, | 10% (EC, JP) | EC, USA, | Not tested |
| Menthyl anthranilate | Meradimate | 5% | USA, AUS | Not tested |
| Octocrylene | Eusolex OCR, 2- | 10% | EC, USA, | Increases |
| | acrylic acid, 2- | | | |
| | ethylhexylester | | | |
| Octyl | Octinoxate, EMC, | 7.5% (US) | EC, USA, | |
| methoxycinnamate | OMC, Ethylhexyl | 10% | AUS, JP | |
| | methoxycinnamate, | (EC, AUS)20% | | |
| | Escalol 557, 2- | (JP) | | |
| | Ethylhexyl- | | | |
| Octyl salicylate | Octisalate, 2-Ethylhexyl | 5% | EC, USA, | Not tested |
| | salicylate, Escalol 587, | (EC, USA, AUS | AUS, JP | |
| Sulisobenzone | 2-Hydroxy-4- | 5% (EC) 10% | EC, USA, | |
| | Methoxybenzophenone- | (US, AUS, JP) | AUS, JP | |
| | 5-sulfonic acid, 3- | | | |
| | Benzoyl-4-hydroxy-6- | | | |
| | methoxybenzenesulfonic | | | |
| | acid, Benzophenone- | | | |
| | 4, Escalol 577 | | | |
| Trolamine salicylate | Triethanolamine | 12% | USA, AUS | Not tested |
| Avobenzone | 1-(4-methoxyphenyl)-3- | 3% (US) 5% | EC, USA, | Not |
| | (4-tert-butyl | (EC, AUS)10% | AUS, JP | Available |
| | phenyl)propane-1,3- | (JP) | | |
| | dione, Butyl methoxy | | | |
| | dibenzoylmethane, | | | |
| | BMDBM, Parsol | | | |
| Ecamsule | Mexoryl SX, | 10% | EC, AUS | Protects |
| | Terephthalylidene | | (US: Approved | against |
| | Dicamphor | | in certain | skin |
| | Sulfonic Acid | | formulations | tumors in |
| | | | up to 3% via | mice |
| | | | New Drug | |
| Titanium dioxide | CI77891 | 25% (No limit | EC, USA, | Not tested |
| Zinc oxide | | 25% (US) 20% | EC, USA, | Protects |
| | | (AUS) | AUS, JP | against |
| | | (EC-25% | | skin |
| | | | | tumors in |
| | | | | mice |
| | | (Japan, No | | |
| | | Limit) | | |

Certain embodiments are directed to pharmaceutical compositions or formulations containing derivatives of camphor. 4-Methylbenzylidene camphor (4-MBC) is an organic camphor derivative that is used in the cosmetic industry for its ability to protect the skin against UV, specifically UV B radiation. As such it is used in sunscreen lotions and other skincare products claiming a SPF value. Its tradenames include Eusolex 6300 (Merck) and Parsol 5000 (DSM).

Other derivatives may include, e.g., salts of 10-camphorsulphonic acid (CSA) that are used in pharmaecutical preparations as an aqueous soluble form of camphor. Camphorsulfonic acid is a while crystalline acid C₁₀H₁₅OSO₃ made by reaction of camphor with sulfuric acid and acetic anhydride. The most frequently found are camphorsulphonates of sodium, codein, piperazine, ephedrine, and ethylmorphine, which are used in tablets, suppositories, oral drops, syrups and injections. The raw material for the preparation of camphorsulphonates for medical use is synthetic camphor, a mixture of optical isomers.

Other camphor derivatives of the invention may include a structural change in the molecule, such as norcamphor. Norcamphor has three methyl groups replaced by hydrogen. Therefore, embodiments of the present invention may include camphor derivatives that may or may not carry a secondary hydroxyl group on the ring.

Structural variations, derivatives, or changes in molecules that would apply to the active agents herein are known in the art. For example, camphor belongs to the group of the bicyclic monoterpenes, certain other monoterpenes belonging to the monocyclic group that are structurally similar are also highly effective in treating the enumerated diseases. It is known in the art that other monoterpenes similar in structure to camphor carry a secondary hydroxyl group. Oxidation to a carbonyl group reduced the activity of the substance drastically, arguing that a hydroxyl group is a structural requirement for efficient activation of TRPV3. Structurally, the position of the hydroxyl group on the ring does not appear to be critical for TRPV3 activation in aromatic substances, but it is relevant for non-aromatic compunds such as dihydrocarveol and (−)-carveol (where the hydroxyl group in the meta position to the isopropyl residue), rather than in the ortho positions as in (−)-isopulegol and (−)-menthol.

Certain other embodiments are directed to pharmaceutical compositions that contain camphor-oil, including camphor white oil, which contains monoterpene constitutents as described herein. Camphor oil can be a natural extract or a synthetic mixture of components (e.g., CAS number 8008-51-3). Table 1 describes terpenes for use in embodiments of the invention.

### A. Form of and Delivery of Pharmacetucial Compositions or Formulations

The pharmaceutical compositions or formulations comprising the active agents may exist in a wide variety of presentation forms, for example: in the form of liquid preparations as emulsions known in the art, or microemulsions gels, oils, creams, milk or lotions, powders lacquers, tablets or make-up, a stick, sprays (with propellent gas or pump-action spray) or aerosols, foams, or pastes.

Embodiments are also directed to cosmetic preparations for the skin comprising one or more active agents including light-protective preparations, such as sun milks, lotions, creams, oils, sunblocks or tropicals, pretanning preparations or after-sun preparations, also skin-tanning preparations, for example self-tanning creams. Of particular interest are sun protection creams, sun protection lotions, and sun protection milk and sun protection preparations in the form of a spray.

Topical formulations of the active agents are preferred. Delivery may occur via dropper or applicator stick, as a mist via an aerosol applicator, via an intradermal or transdermal patch, or by simply spreading a formulation of the invention onto the affected area with fingers. Camphor is well absorbed after inhalation, (if formulated at a dose that is not toxic systemically) or dermal exposure (Baselt and Cravey 1990).

Pharmaceutical compositions of the invention may also include one or more emollients. An emollient is an oleaginous or oily substance, which helps to smooth and soften the skin, and may also reduce its roughness, flaking, cracking or irritation. Typical suitable emollients include mineral oil having a viscosity in the range of 50 to 500 centipoise (cps), lanolin oil, coconut oil, cocoa butter, olive oil, almond oil, macadamia nut oil, aloe extracts such as aloe vera lipoquinone, synthetic jojoba oils, natural sonora jojoba oils, safflower oil, corn oil, liquid lanolin, cottonseed oil and peanut oil. In some embodiments, the emollient is a cocoglyceride, which is a mixture of mono, di and triglycerides of cocoa oil, sold under the trade name of Myritol 331 from Henkel KGaA, or Dicaprylyl Ether available under the trade name Cetiol OE from Henkel KGaA or a C₁₂-C₁₅ Alkyl Benzoate sold under the trade name Finsolv^{TN} from Finetex. Another suitable emollient is DC 200 Fluid 350, a silicone fluid.

Other suitable emollients include squalane, castor oil, polybutene, sweet almond oil, avocado oil, calophyllum oil, ricin oil, vitamin E acetate, olive oil, silicone oils such as dimethylopolysiloxane and cyclomethicone, linolenic alcohol, oleyl alcohol, the oil of cereal germs such as the oil of wheat germ, isopropyl palmitate, octyl palmitate, isopropyl myristate, hexadecyl stearate, butyl stearate, decyl oleate, acetyl glycerides, the octanoates and benzoates of (C₁₂-C₁₅) alcohols, the octanoates and decanoates of alcohols and polyalcohols such as those of glycol and glyceryl, ricinoleates esters such as isopropyl adipate, hexyl laurate and octyl dodecanoate, dicaprylyl maleate, hydrogenated vegetable oil, phenyltrimethicone, jojoba oil and aloe vera extract.

Still other suitable emollients which are solids or semi-solids at ambient temperatures may be used. Such solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate. One or more emollients can optionally be included in the present invention ranging in amounts from about 1 percent to about 10 percent by weight, preferably about 5 percent by weight.

Certain embodiments may be in the form of a topical formulations that include an emulsion. These topical formulations can be in the form of the following:
Cream—Emulsion of oil and water in approximately equal proportions. Penetrates stratum corneum outer layer of skin well.
Ointment—Combines oil (80%) and water (20%). Effective barrier against moisture loss.
Gel—Liquefies upon contact with the skin.
Paste—Combines three agents—oil, water, and powder; an ointment in which a powder is suspended.
Powder—A finely subdivided solid substance

Topical carriers for use in embodiments of the invention are disclosed in REMINGTON: THE SCIENCE AND PRACTICE OF PHARMACY 282-291 (Alfonso R. Gennaro ed. 19th ed. 1995). Suitable gels for use in the invention are disclosed in REMINGTON: THE SCIENCE AND PRACTICE OF PHARMACY 1517-1518 (Alfonso R. Gennaro ed. 19th ed. 1995), U.S. Pat. No. 6,387,383 (issued May 14, 2002); U.S. Pat. No. 6,517,847 (issued Feb. 11, 2003); and U.S. Pat. No. 6,468,989 (issued Oct. 22, 2002. Dow Corning Corp). As used herein, a pharmaceutically acceptable topical carrier is any pharmaceutically acceptable formulation that can be applied to the skin surface for topical, dermal, intradermal, or transdermal delivery of a pharmaceutical or medicament. Pharmaceutical compositions of the invention are typically prepared by mixing a TRPV3 agonist, (e.g., (e.g., camphor, camphor oil, which contains other terpene constituents, and 2-APB or derivatives thereof) with a topical carrier according to well-known methods in the art. The topical carriers include pharmaceutically acceptable solvents, such as a polyalcohol or water; emulsions (either oil-in-water or water-in-oil emulsions), such as creams or lotions; micro emulsions; gels; ointments. Suitable protectives and adsorbents include, but are not limited to, dusting powders, zinc sterate, collodion, dimethicone, silicones, zinc carbonate, aloe vera gel and other aloe products, vitamin E oil, allatoin, glycerin, petrolatum, and zinc oxide.

Topical pharmaceutical compositions of the invention in the form of an emulsion may optionally contain drying agents. Drying agents generally promote rapid drying of moist areas and coats the skin for protection and healing. In particular, it acts to prevent irritation of the involved area and water loss from the skin layer by forming a physical barrier on the skin. Preferred drying agents include calamine; zinc containing drying agents such as zinc oxide, zinc acetate, zinc stearate, zinc sulfate, copper sulfate, kaolin, potassium permanganate, Burow's aluminum solution, talc, starches such as wheat and corn starch, silver nitrate, and acetic acid.

Or these pharmaceutical compositions or formulations may be in the form of an aqueous solution or suspension, preferably, an aqueous solution. Suitable aqueous topical formulations for use in the invention are disclosed in REMINGTON: THE SCIENCE AND PRACTICE OF PHARMACY 1563-1576 (Alfonso R. Gennaro ed. 19th ed. 1995). Other suitable aqueous topical carrier systems are disclosed in U.S. Pat. No. 5,424,078 (issued Jun. 13, 1995); U.S. Pat. No. 5,736,165 (issued Apr. 7, 1998); U.S. Pat. No. 6,194,415 (issued Feb. 27, 2001); U.S. Pat. No. 6,248,741 (issued Jun. 19, 2001); U.S. Pat. No. 6,465,464 (issued Oct. 15, 2002.)

The pharmaceutical compositions of the invention can comprise pharmaceutically acceptable excipients other than emollients, demulcents, and antioxidants such as those listed in REMINGTON: THE SCIENCE AND PRACTICE OF PHARMACY 866-885(Alfonso R. Gennaro ed. 19th ed. 1995; Ghosh, T. K.; et al. TRANSDERMAL AND TOPICAL DRUG DELIVERY SYSTEMS (1997, including, but not limited to, protectives, adsorbents, preservatives, moisturizers, buffering agents, solubilizing agents, skin-penetration agents, and surfactants.

Suitable demulcents include, but are not limited to, benzoin, hydroxypropyl cellulose, hydroxypropyl methylcellulose, and polyvinyl alcohol. Suitable emollients include, but are not limited to, animal and vegetable fats and oils, myristyl alcohol, alum, and aluminum acetate. Suitable preservatives include, but are not limited to, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; antibacterial esters, for example, esters of parahydroxybenzoic acid; and other anti-microbial agents such as chlorhexidine, chlorocresol, benzoic acid and polymyxin.

Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerin, sorbitol, polyethylene glycols, urea, and propylene glycol. Suitable buffering agents for use with the invention include, but are not limited to, acetate buffers, citrate buffers, phosphate buffers, lactic acid buffers, and borate buffers.

Suitable solubilizing agents include, but are not limited to, quaternary ammonium chlorides, cyclodextrins, benzyl benzoate, lecithin, and polysorbates. More specifically, camphor is slightly soluble in water, soluble in alcohol, ether, benzene, acetone, oil of turpentine, glacial acetic acid, chloroform, carbon disulphide, and solvent naphtha and fixed and volatile oils. It is also soluble in aniline, nitrobenzene, tetralin, decalin, methylhexalin, petroleum ether, higher alcohols, concentrated mineral acids, phenol, liquid ammonia and liquid sulphur dioxide.

Embodiments may include skin-penetration agents such as, but are not limited to, ethyl alcohol, isopropyl alcohol, octylphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate); and N-methyl pyrrolidone.

In treating in the form of a dermal-type patch, the pharmaceutical composition is contained within a laminated structure that serves as a drug delivery device to be affixed to the skin. In such a structure, the pharmaceutical composition is contained in a layer, or “reservoir”, underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during active ingredients delivery. Examples of suitable skin contact adhesive materials include, but are not limited to, polyethylenes, polysiloxanes, polyisobutylenes, polyacrylates, polyurethanes, and the like. The particular polymeric adhesive selected will depend on the particular active ingredients, vehicle, etc., i.e., the adhesive must be compatible with all components of the active ingredients-containing composition. Alternatively, the active ingredients-containing reservoir and skin contact adhesive are present as separate and distinct layers, with the adhesive underlying the reservoir which, in this case, may be either a polymeric matrix as described above or it may be a liquid or hydrogel reservoir, or may take some other form.

Targeted drug delivery of the pharmaceutical composition, sometimes called smart drug delivery, is a method of delivering medication to a patient in a manner that increases the concentration of the medication in some parts of the body relative to others. The goal of a targeted drug delivery system is to prolong, localize, target and have a protected drug interaction with the diseased tissue, (e.g., in this case a slow-release transdermal patch). The conventional drug delivery system is the absorption of the drug across a biological membrane, whereas the targeted release system releases the drug in a dosage form. The advantages to the targeted release system is the reduction in the frequency of the dosages taken by the patient, having a more uniform effect of the drug, reduction of drug side-effects, and reduced fluctuation in circulating drug levels.

In an embodiment, the active agents are formulated into liposomes for delivery. Liposomes are microscopic spheres made from fatty materials, predominantly phospholipids. Because of their similarity to phospholipid domains of cell membranes and an ability to carry substances, liposomes can be used to protect active ingredients and to provide time-release properties in medical treatment. Liposomes are made of molecules with hydrophilic and hydrophobic ends that form hollow spheres. They can encapsulate water-soluble ingredients in their inner water space, and oil-soluble ingredients in their phospholipid membranes. Liposomes are made up of one or more concentric lipid bilayers, and range in size from 50 nanometers to several micrometers in diameter. Liposomal formulations have been used for many years to enhance the penetration of topically applied ingredients. Liposomes are made from lecithin, egg or it can be synthesized. These phospholipids can be both hydrogenated and non-hydrogenated. Phosphatidylcholine is extracted from these sources and can be both saturated and unsaturated. Other phospholipids including essential fats like linoleic acid and alpha linolenic acid can be used. Additionally, polyethylene glycol and cholesterol are considered liposomal material because of their lipid structure.

The active agents can be delivered to cancers such as SCCs in the digestive tract for example, or in another location that is difficult to access, e.g. locally in a slow-release formulation or via an implanted pump. Any formulation that delivers the active agents to the intended site is within the scope of this invention. The literature on making such formulations is well known to those in the art.

### B. Dosages and Dosing Frequency

In the experiments herein, camphor, white camphor-oil, dipentene, alpha-pinene and 2-APB were specifically tested. For camphor, which can be toxic if ingested at high enough concentrations, the preferred embodiment is topical administration. It has been shown that topical administration of toxic doses of camphor has little effect on elevating systemic amounts. (See D. Martin, et al., J. Clin. Pharmacol, 2004 October; 44 (10): 1151-57.) The toxicity of the active agents is a factor affected by their pharmacokinetics, formulation and mode of application. Some active agents formulated at high concentrations that would or might be toxic if administered systemically can be administered topically to exposed lesions or they can be administered by local injection to the lesion or the area around the lesion thereby minimizing adverse systemic effects. Alternatively formulations that have a lower concentration of active agent will have lower toxicity and may be administered systemically to treat non-exposed lesions inside the body.

In 1980, the U.S. Food and Drug Administration set a limit of 11% allowable camphor in consumer products, and banned products labeled as camphorated oil, camphor oil, camphor liniment, and camphorated liniment except for “white camphor essential oil” for fear that they would be ingested and become toxic. In the past, when camphor was used medicinally, the oral doses ranged from 120-300 mg (Wade 1977). The parenteral dose range was from 60-200 mg (which not recommended any more).

High doses of camphor (>20%) have been reported to cause skin irritation as well as toxicity when ingested. However, the high doses of camphor or camphor oil described herein for therapeutic use of keratinocyte-derived lesions (going up to 99%) are neither intended for ingestion nor for application on very large areas of the body. Rather, it is intended that camphor and camphor oil for treating keratinocyte derived lesions such as skin cancers or AK is applied locally to affected areas where it could be applied at very high doses. The goal is to treat the patient's lesion including cancer that may need doses of active agents that are formulated at higher than 11%. If the cancer is relatively nonaggressive it may respond to a lower dose than would be needed for treating aggressive high-risk forms of keratinocyte-derived cancers. In the in vivo experiments described herein, therapeutic doses of camphor up to 30% and camphor oil up to 40% (wt/wt) were tested for topical application. Ultimately, any side effects at higher doses may be further managed. It is not expected that high dose formulations of camphor or any other active that is potentially toxic if ingested, would be available over the counter. Rather it would be available as a prescription drug. It may be advisable that very high doses be applied by a physician or under the direction of a physician.

In Canada, amounts of camphor are approved for over the counter formulations of up to 20% for multiple, daily applications in over the counter preparations. Some high doses of camphor may cause necrosis of normal cells along with killing the cancer; however, there is a cost benefit risk to controlling the cancer even at the expense of killing some normal cells. Moreover the dose can be adjusted once the cancer is under control. Normal cells around the periphery of the lesion that may become necrotic can be removed and healthy skin/cells will grow back.

It has also been found in the art that low-dose (i.e., 46.80 mg) dermal application of camphor, resulted in low plasma concentrations of camphor. Even when four and eight patches were applied for 8 hours, there appeared to be relatively low systemic exposure to the potentially toxic. (See D. Martin, et al., J. Clin. Pharmacol, 2004 October; 44 (10): 1151-57.) This supports the position that one trained in dosing and dosing frequency would recognize that higher concentrations of the TRPV3 agonist or derivative thereof may be used, as long any potential side-effects can be separately managed. Note that if camphor white oil is used as the active agent, camphor has been removed or drastically reduced, so higher doses can be used. Concentrations up to 40% wt/wt were tested. Further, active agent(s) can also be injected locally into a lesion, including those that are non-exposed.

Dosages and dosing frequency of all active agents will be determined by a trained medical professional depending on the activity of the biological activity (i.e. efficacy) of the active agent, the dose, the particular formulation (whether for active or systemic administration), and the identity and severity of the dermatologic disorder. Camphor applied to the skin of volunteers as a 20% solution in alcohol produced no significant sensation of irritation or pain at normal skin temperatures (Green 1990). As described above, therapeutically effective amounts of camphor range from about 0.0608-99.5% (wt/wt), preferably in the range of 10%-50% (wt/wt). Therapeutically effective amounts of camphor oil also range from about 0.0608-99.5%, preferably in the range of 10%-50% (wt/wt). By contrast therapeutically effective amounts of 2-APB are much lower: 0.00005-5% (wt/wt). This is because 2-APB activates biological targets at lower concentrations than camphor. 2-APB is also toxic in vivo at high concentrations.

### 7. Kits

In other embodiments of the invention, various kits are also provided. Typically, the kits include a pharmaceutical composition as described herein and instructions for the use of the pharmaceutical composition and dosage regime. The kit can comprise the pharmaceutical composition of the invention in a suitable container with labeling and instructions for use. The container can be, but is not limited to, a dropper or tube. The pharmaceutical composition of the invention can be filled and packaged into a plastic squeeze bottle or tube. Suitable container-closure systems for packaging pharmaceutical compositions of the invention are commercially available for example, from Wheaton Plastic Products, 1101 Wheaton Avenue, Millville, N.J. 08332.

Preferably, instructions are packaged with the formulations of the invention, for example, a pamphlet or package label. The labeling instructions explain how to administer pharmaceutical compositions of the invention, in an amount and for a period of time sufficient to treat or prevent SCC and AK and symptoms associated therewith. Preferably, the label includes the dosage and administration instructions, the topical formulation's composition, the clinical pharmacology, drug resistance, pharmacokinetics, absorption, bioavailability, and contraindications.

### PCR

In certain embodiments, expression of mRNA encoding one or more of the following: TRPV3, TRPC1, TRPV1, and TRPA1 can be determined in a biological sample using known techniques, from which the level of gene expression can be inferred. Levels of mRNA can be quantitatively measured by northern blotting which gives size and sequence information about the mRNA molecules. A sample of RNA is separated on an agarose gel and hybridized to a radioactively labeled probe that is complementary to the target sequence. Or more typically RT-qPCR is used wherein reverse transcription is followed by real-time quantitative PCR (qPCR). Reverse transcription first generates a DNA template from the mRNA; this single-stranded template is called cDNA. The cDNA template is then amplified in the quantitative step, during which the fluorescence emitted by labeled hybridization probes or intercalating dyes changes as the DNA amplification process progresses. With a carefully constructed standard curve, qPCR can produce an absolute measurement of the number of copies of original mRNA, typically in units of copies per nanolitre of homogenized tissue or copies per cell. qPCR is very sensitive. A highly specific RT-qPCR assay for human TRPV3, including well-behaved specific primers, has been developed and is potentially the basis for a diagnostic assay.

The examples disclose the specific methods used to quantitate TRPV3, TRPV1, TRPA1, and TRPC1 mRNA expression, using the specific primers:

1. TRPV3
hGAPDH_1F:
(SEQ. ID. NO. 1)
AAG GGC ATC CTG GGC TAC
hGAPDH_1R:
(SEQ. ID. NO. 2)
AGG GGA GAT TCA GTG TGG TG
hTRPV3_2_F:
(SEQ. ID. NO. 3)
gtcttgaggagcagggagag
hTRPV3_2_R:
(SEQ. ID. NO. 4)
caacccagtcacagcagaag
2. TRPC1
hTRPC1
TRPC1_2_F
(SEQ. ID. NO. 5)
CCT TCT GTT AGT GGC TTT TTG C
TRPC1_2_R
(SEQ. ID. NO. 6)
GCC TAC ATT TGC TGG TCT TCA
3. TRPA1
hTRPA1
TRPA1_3_F
(SEQ. ID. NO. 7)
GAG AGT CCT TCC TAG AAC CAT ATC TGA
TRPA1_3_R
(SEQ. ID. NO. 8)
CAT GAG GAC AAT TGG GAC AAA TAT T
4. TRPV1
hTRPV1
TRPV1_F
(SEQ. ID. NO. 9)
GTT TGG GGG TGT TGG TGT T
TRPV1_R
(SEQ. ID. NO. 10)
CCT TTG GGA TGT GGT TCT GT

GenBank Accession Numbers:

### 1. TRPV3

TrpV3 Mouse sequences:
Gene-TRPV3
Gene ID: 246788
NC_{—}000077.6
TrpV3 mRNA-
NM_{—}145099.2

TRPV3 human sequences:
Gene-TRPV3
Gene ID: 162514
NG_{—}032144.2
mRNA-
NM_{—}001258205.1

### 2. TRPV1

TrpV1 Mouse sequences:

Gene-TRPV1
Gene ID: 193034
NC_{—}000477
TrpV1 mRNA-
NM001001445.1

TRPV1 human sequences:
Gene-TRPV1
Gene ID: 7442
NG_{—}029716
mRNA-
NM018727.5

### 3. TRPA1

TrpA1 Mouse sequences:
Gene-TRPA1
Gene ID: 277328
NC_{—}000067
TrpA1 mRNA
NM177781.4

TRPA1 human sequences;
Gene-TRPA1
Gene ID: 8989
NC000008.10_{—}
mRNA-
NM007332.2

### 4. TRPC1

TrpC1 Mouse sequences:
Gene-TRPC1
Gene ID: 22063
NC_{—}000075
TrpA1 mRNA
NM01164312

TRPC1 human sequences;
Gene-TRPC1
Gene ID:7220
NG_{—}030369.1
mRNA-
NM001251845.1

The qPCR cycling conditions are as follows:
48 C for 30 min
95 C for 10 min
95 C 15 sec
60 C 1 min
Repeat the last two steps 40 times.

Target nucleic acids are amplified to obtain amplification products. Suitable nucleic acid amplification techniques are well known to a person of ordinary skill in the art, and include polymerase chain reaction (PCR) as for example described in Ausubel et al., Current Protocols in Molecular Biology (John Wiley & Sons, Inc. 1994-1998) (and incorporated herein). The most commonly used nucleic acid amplification technique is the polymerase chain reaction (PCR). PCR is well known in this field and comprehensive description of this type of reaction is provided in E. van Pelt-Verkuil et al., Principles and Technical Aspects of PCR Amplification, Springer, 2008. PCR is a powerful technique that amplifies a target DNA sequence against a background of complex DNA. If RNA is to be amplified (by PCR), it must be first transcribed into cDNA (complementary DNA) using an enzyme called reverse transcriptase. Afterwards, the resulting cDNA is amplified by PCR. PCR is an exponential process that proceeds as long as the conditions for sustaining the reaction are acceptable. The components of the reaction are: (i). pair of primers—short single strands of DNA with around 10-30 nucleotides complementary to the regions flanking the target sequence; (ii). DNA polymerase—a thermostable enzyme that synthesizes DNA; (iii). deoxyribonucleoside triphosphates (dNTPs)—provide the nucleotides that are incorporated into the newly synthesized DNA strand; and (iv). buffer—with MgCl₂ to provide the optimal chemical environment for DNA synthesis.

PCR typically involves placing these reactants in a small tube (˜10-50 μl) containing the extracted nucleic acids. The tube is placed in a thermal cycler; an instrument that subjects the reaction to a series of different temperatures for varying amounts of time. The standard protocol for each thermal cycle involves a denaturation phase, an annealing phase, and an extension phase. The extension phase is sometimes referred to as the primer extension phase. In addition to such three-step protocols, two-step thermal protocols can be employed, in which the annealing and extension phases are combined. The denaturation phase typically involves raising the temperature of the reaction to 90-95° C. to denature the DNA strands; in the annealing phase, the temperature is lowered to ˜50-60° C. for the primers to anneal; and then in the extension phase the temperature is raised to the optimal DNA polymerase activity temperature of 60-72° C. for primer extension. This process is repeated cyclically around 20-40 times.

There are a number of variants to the standard PCR protocol such as multiplex PCR, linker-primed PCR, direct PCR, tandem PCR, real-time PCR and reverse-transcriptase PCR, amongst others, which have been developed for molecular diagnostics.

Multiplex PCR uses multiple primer sets within a single PCR mixture to produce amplicons of varying sizes that are specific to different DNA sequences. By targeting multiple genes at once, additional information may be gained from a single test-run that otherwise would require several experiments. Optimization of multiplex PCR is more difficult though and requires selecting primers with similar annealing temperatures, and amplicons with similar lengths and base composition to ensure the amplification efficiency of each amplicon is equivalent.

Linker-primed PCR, also known as ligation adaptor PCR, is a method used to enable nucleic acid amplification of essentially all DNA sequences in a complex DNA mixture without the need for target-specific primers. The method firstly involves digesting the target DNA population with a suitable restriction endonuclease (enzyme). Double-stranded oligonucleotide linkers (also called adaptors) with a suitable overhanging end are then ligated to the ends of target DNA fragments using a ligase enzyme. Nucleic acid amplification is subsequently performed using oligonucleotide primers which are specific for the linker sequences. In this way, all fragments of the DNA source which are flanked by linker oligonucleotides can be amplified.

Direct PCR describes a system whereby PCR is performed directly on a sample without any, or with minimal, nucleic acid extraction. It has long been accepted that PCR reactions are inhibited by the presence of many components of unpurified biological samples, such as the heme component in blood. Traditionally, PCR has required extensive purification of the target nucleic acid prior to preparation of the reaction mixture. With appropriate changes to the chemistry and sample concentration, however, it is possible to perform PCR with minimal DNA purification, or direct PCR. Adjustments to the PCR chemistry for direct PCR include increased buffer strength, the use of polymerases which have high activity, ability to process, and additives.

Tandem PCR utilizes two distinct rounds of nucleic acid amplification to increase the probability that the correct amplicon is amplified. One form of tandem PCR is nested PCR in which two pairs of PCR primers are used to amplify a single locus in separate rounds of nucleic acid amplification. The first pair of primers hybridize to the nucleic acid sequence at regions external to the target nucleic acid sequence. The second pair of primers (nested primers) used in the second round of amplification bind within the first PCR product and produce a second PCR product containing the target nucleic acid, that will be shorter than the first one. The logic behind this strategy is that if the wrong locus were amplified by mistake during the first round of nucleic acid amplification, the probability is very low that it would also be amplified a second time by a second pair of primers and thus ensures specificity.

Real-time PCR, or quantitative PCR, is used to measure the quantity of a PCR product in real time. By using a fluorophore-containing probe or fluorescent dyes along with a set of standards in the reaction, it is possible to quantitate the starting amount of nucleic acid in the sample. This is particularly useful in molecular diagnostics where treatment options may differ depending on the pathogen load in the sample.

### RT-PCR

Typically DNA sequences are amplified, although in some instances RNA sequences can be amplified or converted into cDNA, such as by using RT PCR. Reverse-transcriptase PCR (RT-PCR) is used to amplify DNA from RNA. Reverse transcriptase is an enzyme that reverse transcribes RNA into complementary DNA (cDNA), which is then amplified by PCR. RT-PCR is widely used in expression profiling, to determine the expression of a gene or to identify the sequence of an RNA transcript, including transcription start and termination sites. It is also used to amplify RNA viruses such as human immunodeficiency virus or hepatitis C virus. “cDNA” or “complementary DNA” is DNA synthesized from a messenger RNA (mRNA) template in a reaction catalyzed by the enzyme reverse transcriptase and the enzyme DNA polymerase. Complementary base sequences are those sequences that are related by the base-pairing rules. In DNA, A pairs with T and C pairs with G. In RNA, U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently.

A reverse transcriptase PCR™ amplification procedure may be performed when the source of nucleic acid is fractionated or whole cell RNA. Methods of reverse transcribing RNA into cDNA are well known (see Sambrook et al., 1989). Alternative methods for reverse polymerization utilize thermostable DNA polymerases. These methods are described in WO 90/07641. Representative methods of RT-PCR are described in U.S. Pat. No. 5,882,864.

Other embodiments are set forth in the summary of the invention, or described in the pharmaceutical composition section below.

### 8. Examples

The invention is illustrated herein by the experiments described by the following examples, which should not be construed as limiting. The contents of all references, pending patent applications and published patents, cited throughout this application are hereby expressly incorporated by reference. Those skilled in the art will understand that this invention may be embodied in many different forms and should not be construed as limited to the embodiments set forth herein. Rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated.

### Example 1

### Methods and Materials for In Vitro Experiments

Cell Culture.

Normal human keratinocytes were isolated from human foreskins by undergoing two enzymatic dissociation steps, first in dispase then in trypsin. Cells were cultured in EpiLife supplemented with human keratinocyte growth supplement (HKGS; Invitrogen). For all experiments with undifferentiated cells, keratinocytes were harvested or images at ˜75% confluency. Differentiation was induced by adding CaCl₂ to the EpiLife media to reach a final [Ca2+] of 1.2 mM. Cells were typically cultured for 3 days under differentiation conditions before they were assayed. Keratinocytes were cultured for <5 passages. Purified Camphor (CAS #76-22-2), 2-APB (Tocris) or vehicle (1% EtOH)-containing media were used as indicated.

Organotypic human skin cultures and primary SCC lines were isolated from human SCCs and cultured as previously described (Bachelor et al., 2011). In brief, human fibroblasts were seeded in a collagen matrix on a 70 μm filter insert and incubated at 37° C. for 5-7 days. Human keratinocytes or SCC cells were then seeded on the apical surface of the fibroblast-collagen matrix and cultured for 2 days submerged in media to allow monolayer formation. The media was then removed from the apical surface (raising), exposing the keratinocytes or SCC cells to air. The cultures were maintained in this fashion for up to 14 days, and the media to the fibroblasts below was changed every 2-3 days. At the time of raising, 50 μM 2-APB or vehicle (1% EtOH) was added to the media.

Histology.

Sections were fixed in 4% paraformaldehyde, paraffin embedded and sectioned at 8 μm. Hematoxylin and Eosin (H&E) stained sections were imaged with a bright field microscope equipped with a 10× (0.3 NA) and 20× (0.4 NA) objectives and an Axiocam color CCD camera (Zeiss AxioObserver.Z1). Epidermal thickness was assessed by measuring the nucleated layers with ImageJ software (NIH ImageJ). Number of invading cells was determined by counting the number of round nuclei below the basement membrane per 10× field (fibroblast nuclei are flatten and were excluded). Four non-serial paraffin sections per experiment were examined. Six random frames per section were quantified.

Proliferation Assays.

Cells were treated with 2-APB or camphor for 24 hours at 37° C., exposed to EdU for 45 min, fixed and stained with DAPI for cell counting. EdU Click-It assays (Invitrogen) were used to assess cell-cycle entry. The cells were then fixed and stained with DAPI. Three fields per well were assayed via a 20× objective and there were 4 wells per treatment in each experiment. EdU=5-ethynyl-2′-deoxyuridine.

Ratiometric Calcium Imaging.

Human keratinocytes and SCC cells were washed in Ringer's solution (in mM: NaCl 140, KCl 5, D-glucose 10, HEPES 10, CaCl₂ 2, MgCl₂ 2, pH 7.2) and loaded with 5 μM fura-2AM and 1 μM pluronic (Invitrogen) for 30 minutes in the dark. During live-cell imaging, saturating concentrations of TRP agonists (10 mM Camphor, 100 μM 2-APB 1 μM Capsaicin, 300 μM Mustard oil or 3 μM 4αPDD) in Ringer's were used. Cells were imaged for 20 s to establish a baseline and then acutely treated with each agonist for 60 seconds. Imaging was performed on an Olympus IX81 microscope with a 20× (NA 0.17) objective and a Hamamatsu ORCA-R2 camera. 340- and 380-nm excitation and 540-nm emission filters were used to capture fura-2 fluorescence. Data are expressed as a ratio of fluorescent signals (340/380). All data acquisition was performed in MetaFluor. Data analysis was performed with custom algorithms in IgorPro. Cells with baseline levels ±2 SD above the mean were excluded from analysis as unhealthy. Responders were designated as cells whose 340/380 ratio was ≧20% above baseline.

qPCR.

RNA was isolated using RNeasy Kit (Qiagen) and reversed transcribed. Four technical replicates were run for each primer set and cDNA sample using SYBR Green (Applied Biosystems) for quantitative readout. Complementary DNA was synthesized with oligo-dT primers and SuperScript III (Invitrogen). Primers were generated in Primer3. Primer pairs were optimized for qPCR and validated in control specimens. Standardized SYBR green amplification protocols were used on the StepOnePlus ABI machine as suggested by the distributor (Applied Biosystems). Melting curves were generated for all products to confirm a single amplicon for each product. To determine gene expression in each sample, cycle thresholds (C_{T}) of the gene of interest were normalized to the reference gene GAPDH (ΔC_{T}). Fold change was determined using the ΔΔC_{T} method where vehicle or growth conditions were used as the calibrator (ΔΔC_{T}=[(C_{T}(target gene)−C_{T}(reference gene)]−[C_{T}(calibrator)]−C_{T}(reference gene)]. All gene amplifications were performed in quadruplicate.

Statistics.

Experimental replicates were performed using normal human epidermal keratinocytes independently isolated from different human neonatal foreskin specimens (n=14 specimens). For each independent experiment, 3-8 technical replicates were performed. Twelve individual SCC and two normal skin biopsies were obtained. Four technical replicates were performed on each biopsy. Organotypic cultures were carried out in triplicate. Data are expressed as means±SEMs unless noted. Statistical significance was assessed with Chi-square analysis, unpaired two-tailed Student's t tests or two-way ANOVA with Bonferroni post hoc analysis (GraphPad Prism).

### Example 2

### Responses to TRP-Channel Agonists are Potentiated in Differentiating Human Keratinocytes In Vitro

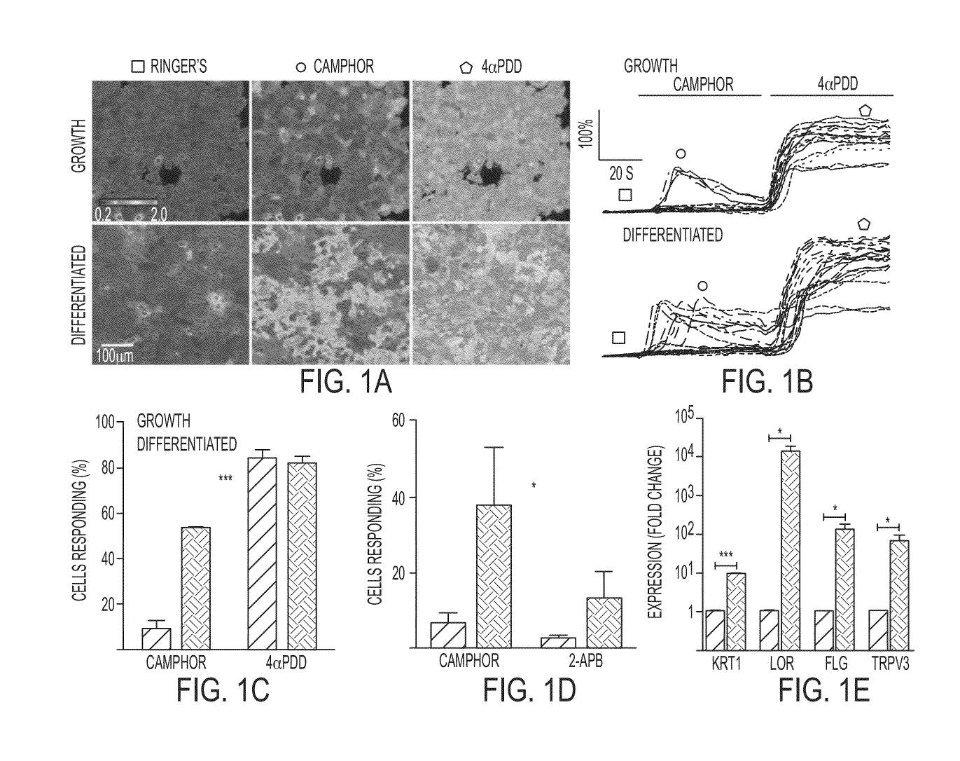

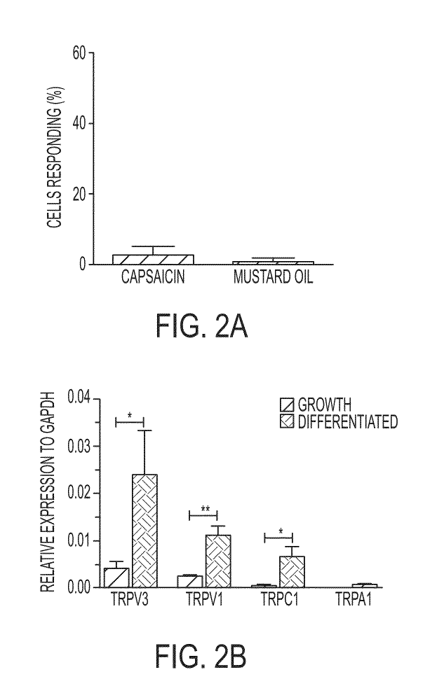

TRP channels are selectively activated by endogenous and exogenous ligands at micromolar to millimolar concentrations. Since TRP channel activity increases over this range to allow graded Ca²⁺ influx, receptor activity levels can be monitored by live-cell Ca²⁺ imaging. A ratiometric calcium indicator (Fura-2) was employed to determine whether a panel of TRP-channel agonists elicited cytoplasmic calcium increases in human epidermal keratinocytes. (FIG. 1A-1E, FIG. 2A-2B, and Table 3).

Normal human keratinocytes were cultured in low-calcium media to promote proliferation or in 1.2-mM calcium media to induce differentiation. For live-cell imaging, keratinocytes were then washed and bathed in Ringer's solution containing 2 mM calcium so that calcium signals could be directly compared between cell populations. Consistent with previous reports, it was determined that addition of a selective TRPV4 agonist, 4α-phorbol-12,13 didecanoate (4αPDD), elicited robust increases in intracellular Ca²⁺ in >70% of keratinocytes cultured in growth or differentiation conditions (Kida et al., 2012). By contrast, the proportion of cells showing Ca²⁺ increases in response to 10 mM camphor, which activates TRPV3 (Moqrich et al., 2005; Vanden abele et al., 2003) and TRPV1 (Xu et al., 2005) and inhibits TRPA1 (Sawada et al., 2007; Vanden Abeele et al., 2003) was 5.7-fold greater in differentiated keratinocytes than in proliferating keratinocytes FIG. 1A-1C and Table 4 _{χ}2<0.0001). The magnitude of camphor-evoked responses was also significantly larger in differentiated keratinocytes (differentiated=78±1% increase over baseline, growth=55±1% increase, P<0.001, Student's t test).

TABLE 4
Quantification of keratinocyte responses to TRP channel agonists.
Each compound is listed and the percentage of responsive keratinocytes. Responders are
designated as cells that had an increase in F340:F380 signal ≧20%.
Data represent Mean ± SEM or SD.

| | | % Responders | | % Responders | |
|---|---|---|---|---|---|
| | | (undifferentiated) | | (differentiated) | |
| Agonist | Concentration | Mean ± SEM( ) or SD(\hat{ }) | N = | Mean ± SEM | N = |
| 4αPDD | 3 μM | 84.8 ± 1.6% | 3 exp* | 70.8 ± 11.1% | 3 exp |
| Camphor | 10 mM | 6.5 ± 2.8% | 3 exp | 37.5 ± 15.7% | 3 exp |
| 2-APB | 100 μM | 2.5 ± 0.9% | 3 exp | 13.0 ± 7.2% | 3 exp |
| Capsaicin | 1 μM | 2.3 ± 2.6%\hat{ } | 13 wells | not determined | |
| Mustard oil | 300 μM | 0.6 ± 1.2%\hat{ } | 17 wells | not determined | |

To narrow camphor's molecular target, additional agonists of TRPV1, TRPA1, and TRPV3 were tested to determine whether these channels were functional in normal human epidermal keratinocytes. Keratinocytes displayed little or no change in cytoplasmic calcium in response to saturating concentrations of TRPV1 or TRPA1 agonists (TRPV1, 1 μM capsaicin=2.3+/−2.6% of cells responding; TRPA1, 300 μM mustard oil=0.6+/−1.2% of cells responding; FIG. 2A and Table 4). Capsaicin and mustard oil are high efficacy agonists that robustly activate their respective TRP-channel targets (Table 3). Therefore, proliferating human keratinocytes have only low levels of functional TRPV1 or TRPA1 in vitro.

These results were confirmed using a higher affinity TRPV3 agonist that does not target TRPV1 or TRPA1, 2-APB, which is structurally distinct from camphor and activates TRPV3 at a different binding site (FIG. 1D; (Chung et al., 2004a; Hu et al., 2009). Like camphor, 100 μM 2-APB elicited cytoplasmic calcium increases in keratinocytes and these 2-APB-evoked responses were upregulated in differentiated keratinocytes. The proportion of cells responding to either TRPV3 agonist was significantly increased in differentiated keratinocytes (FIG. 1D, cell type P<0.05, two-way ANOVA). Although 2-APB has been reported to block TRPC1 channels (Chung et al., 2004a), TRPC1 inhibition cannot account for these results because channel block will cause cytoplasmic calcium decreases rather than the calcium increases observed. The 2-APB-evoked Ca²⁺ increases observed were consistent with Ca²⁺-channel activation rather than inhibition. It was determined that calcium signals elicited by camphor and 2-APB were increased in human keratinocytes upon differentiation.

It was then determined whether these increased 2-APB or camphor-evoked activity reflect enhanced TRP-channel gene expression. Quantitative PCR (qPCR) demonstrated that TRPV3 transcripts were enriched 66-fold in differentiated keratinocytes compared with those cultured in growth conditions (FIG. 1E, FIG. 2B; P=0.04). TRPV1 and TRPC1, which were expressed at lower levels than TRPV3, were also upregulated in differentiated keratinocytes (FIG. 2B). By contrast, TRPA1 was only amplified at detectable levels in one out of three biological replicates. TRP channel upregulation was accompanied by increased expression of well-established early differentiation markers including keratin-1 (KRT1), loricrin (LOR), filaggrin (FLG; FIG. 1E; (Li et al., 1995). Therefore, differentiation stimulates TRP-channel gene expression in normal keratinocytes, which results in an increase in both the proportion of keratinocytes responding to 2-APB and camphor and the magnitude of these cytoplasmic calcium signals.

### Example 3

### Camphor and 2-APB Exert, Pleotropic Effects on Human Keratinocyte Behavior In Vitro

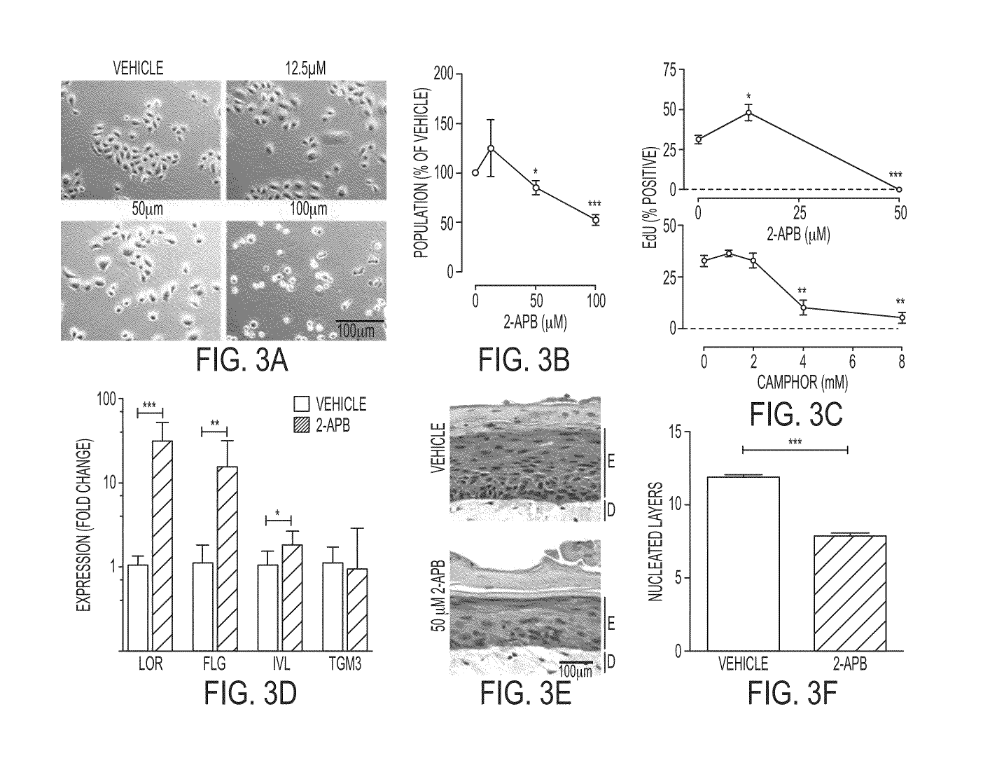

As Ca²⁺ triggers the commitment switch from proliferation to differentiation in keratinocytes, it was reasoned that increasing intracellular calcium by incubating keratinocytes in TRP agonists might be sufficient to induce this cell-fate switch in low-calcium growth media. To test this hypothesis, normal human keratinocytes were cultured for 24 hours under low-Ca²⁺ conditions in the presence of low (12.5 μM), half-maximal (50 μM) and saturating (100 μM) concentrations of 2-APB (FIG. 3A) or camphor (0-8 mM) (FIG. 3E). These TRP agonists were chosen because they reliably elicited intracellular calcium signaling in normal human keratinocytes (FIG. 1A-1E).

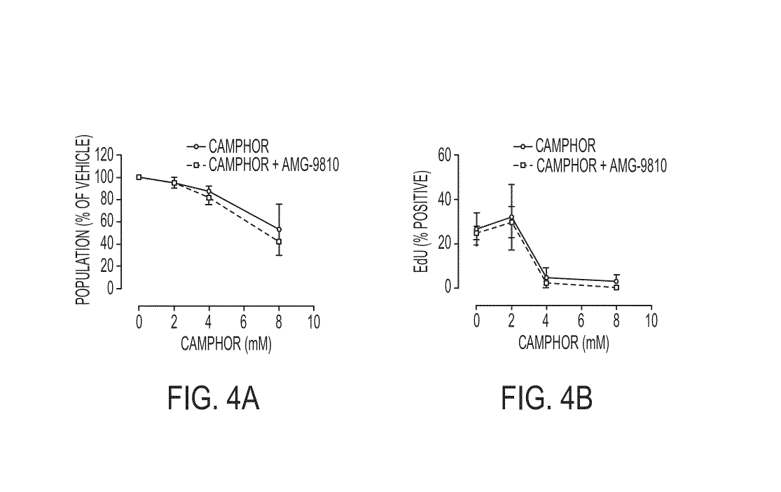

First, cellular morphology, cell counts and cell cycle entry were assessed as evidenced by (5-ethynyl-2′-deoxyuridine) incorporation (FIG. 3A-3C. At 12.5 or 50 μM 2-APB, keratinocyte morphology was indistinguishable from vehicle-treated cells. At 12.5 μM 2-APB, a population increase was noted in one set of primary keratinocytes (325+/−5% compared with vehicle-treated controls, n=4 replicates, P<0.0001; Student's t test) and a significant increased in EdU incorporation was also noted across all experiments (FIG. 3B; P<0.05, Student's t test). Low concentrations of 2-APB promote keratinocytes proliferation. At 50 μM, 2-APB caused a slight reduction in cell number and completely inhibited EdU incorporation, indicating cell-cycle arrest (FIG. 3A-3C). Similar effects were observed with 4-8 mM camphor (FIG. 3C). As camphor can activate both TRPV3 and TRPV1 channels, these experiments were repeated in the presence of a specific TRPV1 antagonist, AMG-9810 (Gavva et al., 2005). TRPV1 inhibition did not alter camphor's effects on keratinocyte proliferation (FIG. 4A-4B). Therefore, camphor-evoked cell-cycle arrest is unlikely to be mediated through TRPV1-dependent mechanisms. At 100 μM, 2-APB induced necrotic morphological changes and a loss of cell numbers, consistent with a previous report that saturating 2-APB concentrations induce keratinocyte cytotoxicity (FIG. 3A-3B) and Table 3; Borbiro et al., 2011).

2-APB exerts activity-dependent, pleotropic effects on keratinocyte behavior. Moreover, they identify 2-APB and camphor concentrations (50 μM and 4-8 mM, respectively) that arrest proliferation without inducing necrotic changes in normal human keratinocytes. As proliferation arrest is a hallmark of cell-fate commitment, this observation is consistent with the hypothesis that TRP agonists can induce the fate switch from proliferation to differentiation in human keratinocytes.

It was then determined whether moderate TRPV3 activation, specifically, incubation with 50 μM 2-APB, was sufficient to promote expression of keratinocyte differentiation genes. In low-calcium growth media, LOR and FLG transcript levels were induced more than 15-fold in keratinocytes treated for 24 hours with 50 μM 2-APB compared with vehicle controls (FIG. 3D). This observation is consistent with reduced LOR protein observed in TRPV3 knock-out mice. By comparison, it was observed that little change in expression of the late-stage differentiation genes involucrin (IVL) and transglutaminase 3 (TGM3; FIG. 3D). This might be due to the brief 2-APB incubation period, as late differentiation markers are typically not expressed in culture until 48 hours after the induction of keratinocyte differentiation (Hennings et al., 1980). Alternatively, 2-APB might preferentially regulate early differentiation genes. Collectively, it was determined that constitutive treatment with 50 μM 2-APB in low-calcium media was sufficient to commit human keratinocytes to a differentiated state.

Since 50 μM 2-APB induced proliferation arrest and keratinocyte differentiation in vitro, it was predicted that 2-APB might alter epidermal stratification. This prediction was tested with human organotypic 3D skin equivalent models (Commandeur et al., 2009; Obrigkeit et al., 2009). Human keratinocytes were seeded on dermal matrices, allowed to form a monolayer, raised to an air-liquid interface to induce stratification and then treated for 7 days with either vehicle or 50 μM 2-APB. 2-APB-treated skin equivalents displayed a one-third reduction in nucleated epidermal layers (FIG. 3E-FIG. 3F). These data extended observations in two-dimensional keratinocyte cultures by demonstrating that constitutive exposure to 2-APB altered human keratinocyte behavior in a stratifying epidermis.

### Example 4

### TRP-Channel Expression is Dysregulated in High-Risk Human Cutaneous SCCs In Vitro

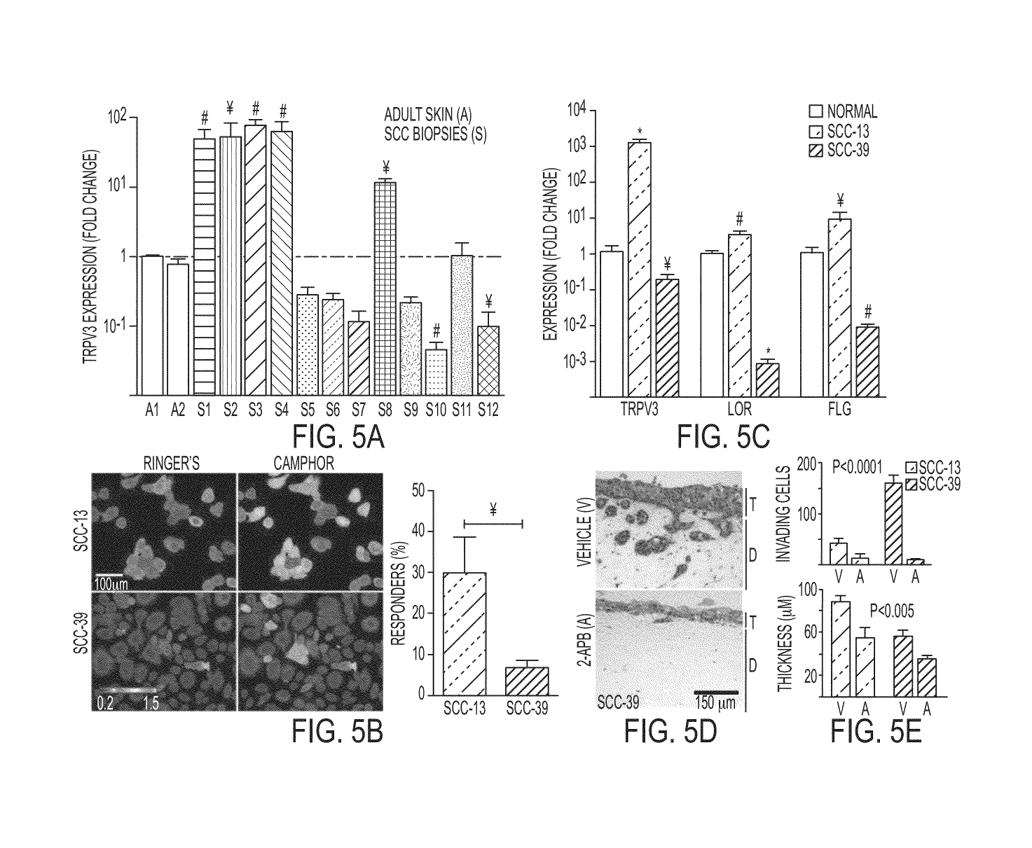

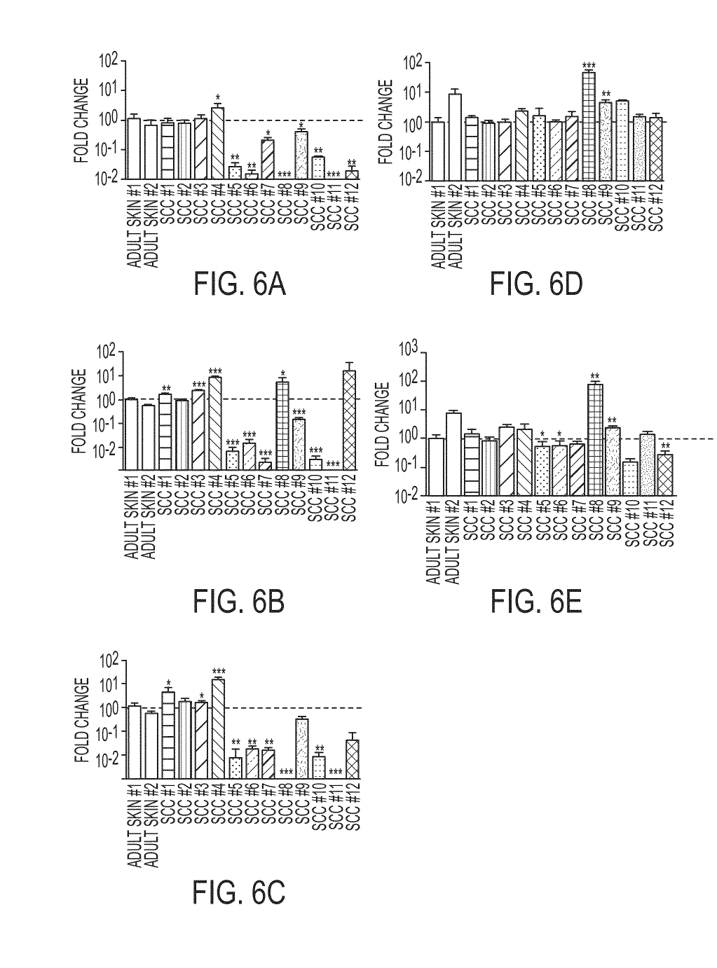

SCC is a pathological condition marked by perturbed keratinocyte differentiation and alterations in EGFR signaling. As TRPV3 potently activates EGFR signaling in mouse keratinocytes (Cheng et al., 2010; Pan et al., 2011; Tajeddine & Gailly, 2012), it was reasoned that TRP channel expression might be dysregulated in SCC. To test this hypothesis, expression levels of TRP-channel genes were quantified in high-risk human SCC specimens (FIG. 5A and FIG. 6A-6E). It was determined that TRPV3, TRPV1, TRPA1, and TRPC1 gene expression levels were significantly dysregulated in >60% of SCC of SCC biopsies compared with normal human skin. FIG. 5A and FIG. 6A-6E). By comparison, expression of signature SCC biomarkers Cyclin D1 (CCND1) and EDFR (Hardisson, 2003) was altered in only 17% and 41% of patient specimens, respectively (FIG. 6A-6E). Therefore, TRP channel expression is dysregulated in a majority of human SCCs examined.

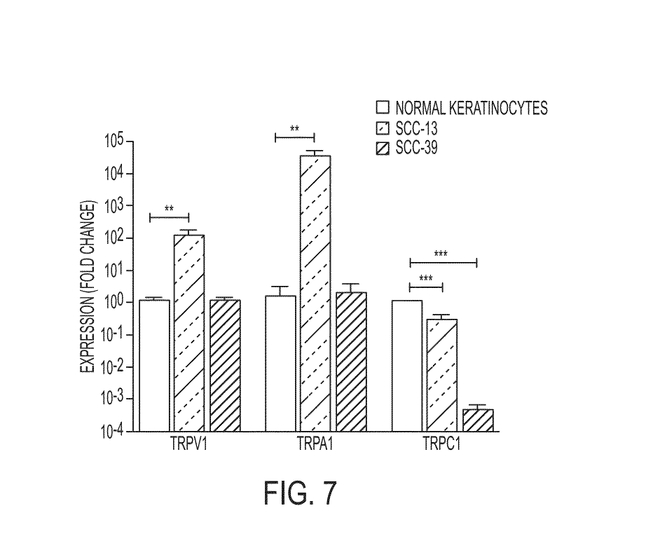

To ask whether TRP channels are functional in human SCC keratinocytes, two cell lines derived from human SCC tumors (SCC-13 and SCC-39; (Bachelor et al., 2011); FIG. 5B were assayed with live-cell calcium imaging. Camphor-evoked cytoplasmic calcium signals were increased in SCC-13 cells (FIG. 5B) compared with proliferating keratinocytes (FIG. 1C). Consistent with enhanced camphor-evoked responses, expression levels of TRPV3, LOR, and FLG were augmented in SCC-13 cells (FIG. 5C). Expression levels of TRPV1, TRPC1, and TRPA1 were also increased (FIG. 7). By contrast, SCC-39 cells showed significantly fewer camphor-evoked calcium responses compared with SCC-13 cells (FIG. 5B). Similarly, SCC-39 cells showed reduced expression levels of TRPV3, TRPV1, TRPC1, TRPA1, LOR and FLG compared with normal keratinocytes and SCC-13 cells (FIG. 5B and FIG. 7). Together, these results illustrated that human SCCs preserve the coordinated pattern of expression between TRP channels and differentiation genes that we observe in normal human keratinocytes (FIG. 1E). Moreover, their responses to camphor mirror their respective levels of TRP channel expression.

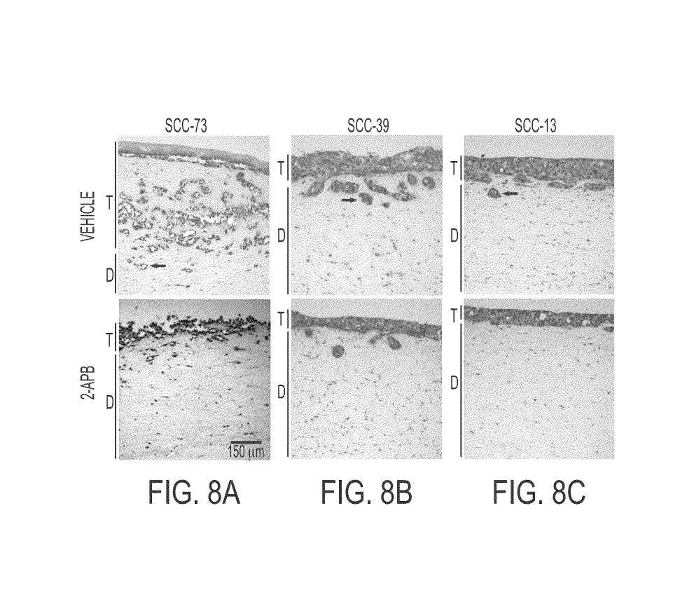

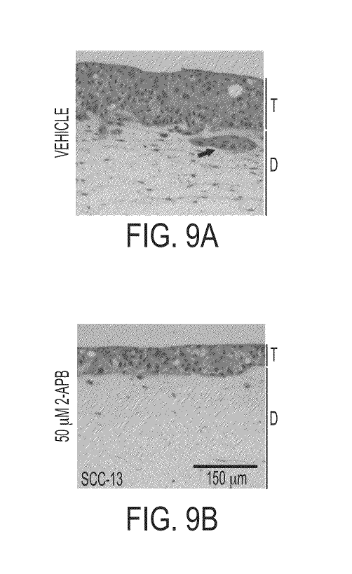

2-APB reduces SCC tumor growth and invasion in human preclinical models. As TRP-channels are expressed in human SCC keratinocytes and TRP agonists arrest keratinocyte proliferation, it was reasoned that TRP agonists might be candidates for SCC-targeted therapy. To test this notion, organotypic human skin cultures were seeded with SCC cells. SCC-39 showed significantly more cells invading into the dermis (FIG. 5D-5E and FIG. 8B) than SCC-13 (FIG. 4; P=0.003, Student's t test). SCC-39's enhanced invasiveness was consistent with its less differentiated molecular signature (FIG. 3C); (Ratushny et al., 2012). Conversely, SCC tumor formation apical to the dermis was significantly larger in SCC-13 compared with SCC-39 (FIG. 5E, FIG. 8C, and FIG. 9A-9B). A third cell line, SCC-73, displayed a much more dysplastic nature with no obvious basement membrane formation (FIG. 8A). Thus, in 3D organotypic cultures, these SCC cell lines recapitulate a range of tumor behaviors observed in vivo.

2-APB treatment dramatically reduced tumorigenesis in organotypic cultures seeded with each of these three SCC cell lines. In SCC-39 organotypic cultures, 2-APB treatment inhibited cell invasion by >93% compared with vehicle-treated cultures (P<0.0001, Bonferroni post hoc, FIG. 5E). Tumor formation above the dermis in SCC-13 and SCC-39 was reduced by >37% (P<0.01, Bonferroni post hoc, FIG. 5E). 2-APB also dramatically reduced the invasive nature and growth of SCC-73 tumor cells in human 3D cultures (FIG. 8A, N=3 culture per treatment). Together, these human preclinical models support the conclusion that 2-APB reduces SCC tumor size and dermal invasion in vitro.

### Example 5

### Methods and Materials for In Vivo Experiments

Animals. Animal use was conducted according to guidelines from the National Institutes of Health's Guide for the Care and Use of Laboratory Animals and the Institutional Animal Care and Use Committee of Columbia University Medical Center.

Experimental Design.

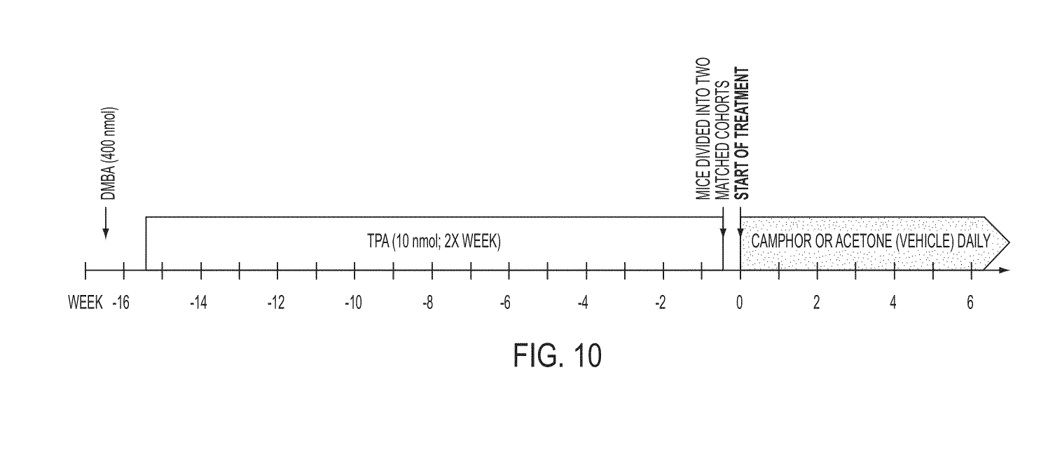

The overall experimental design is summarized in FIG. 10.

Chemical Carcinogenesis Model.

Genetically identical, age-matched, female FVB mice (N=24; Jackson Laboratories) were used because they are susceptible to skin carcinogenesis with this model. All topical agents were applied to shaved dorsal skin. To ensure that topical agents permeated the skin barrier, all topical agents were dissolved in acetone. Mice (6-7 weeks of age) were shaved once on the dorsal surface with electric clippers. After two days, animals were checked to ensure that they did not show signs of hair regrowth, confirming that they were in the telogen (resting) stage of the hair cycle. Each animal received a single topical application of 400 nmol DMBA in 200 μl acetone to initiate tumorigenesis. One week later, mice received twice weekly applications of 10 nmol TPA in 200 μl acetone for a period of 15 weeks. All mice developed multiple skin tumors at 15 weeks with this protocol (median: 14 tumors per mouse, range: 3-30 per mouse).

Tumor Quantification.

Tumor number and location were documented weekly. Tumor diameters were estimated using digital calipers. A lesion was classified as a precancerous lesions based on its appearance as a non-ulcerated, fleshy pedunculated or sessile wart-like mass with a diameter in any dimension ≧2 mm that persisted for at least one week. In this chemical carcinogenesis model, a subset of high-risk precancerous lesions will convert to malignant SCCs. Lesions were classified as malignant SCCs based on the following criteria: 1) conversion from a fleshy lesion to a flattened circular growth with a depressed center, 2) spontaneous ulceration (Allen et al, 2003; PMID: 12566297). Mice were monitored daily and euthanized when they reached one of the following IACUC-approved endpoints: 1) a tumor >20 mm in diameter in any dimension, 2) tumor ulceration that penetrates below the dermis, leading to loss of skin barrier, 3) signs of anemia for ≧24 h, 4) tumor burden that interferes with eating or drinking (e.g., on the mouth), or 5) gross appearance indicating distress (hunched posture, lethargy, persistent recumbence). Tissue from euthanized animals was harvested for histology and molecular analysis. Lungs and lymph nodes were examined for SCC-derived metastases.

Camphor Oil or Vehicle (Acetone) Treatment.

Three days after the last TPA treatment, mice were randomly assigned to Camphor oil (synthetic camphor white oil CAS#8008-51-3 Sigma Aldrich) and control treatment groups, which were matched for total precancerous lesion burden (N=162 lesions in 12 mice per group). Mice assigned to the Camphor oil group were treated daily with topical 20% (wt/wt) camphor white oil in acetone (wt/wt; 400 μl applied drop wise to precancerous leision and SCC lesions; CAS 8008-51-3; Sigma catalog # W223115; lot MKBG8153V). Mice assigned to the control (acetone vehicle) group were treated daily with topical acetone (400 μl applied drop wise to lesions).

Histopathological Analysis.

Skin tumors were surgically excised from euthanized mice, fixed and embedded in paraffin wax blocks. Histological tumor sections were generated from paraffin blocks and stained with hematoxylin and eosin for microscopic examination. Criteria used for verifying skin lesions as precancerous lesions or SCCs were as described (Bogovski, P. Tumours of the skin. In: V. Turusov and U. Mohr (eds.). Pathology of Tumours in Laboratory Animals).

Data Analysis and Statistics.

Four measures of tumor burden and survival were assessed: 1) mean number of benign tumors (precancerous lesions) per mouse, 2) total number of malignant SCCs per group, 3) tumor incidence, expressed as the percentage of mice with pre-malignant or malignant lesions, and 4) time to experimental endpoint for each mouse. Data are expressed as mean±SEM unless noted. Statistical significance was assessed with two-way ANOVA followed by Bonferroni post hoc analysis when appropriate (GraphPad Prism version 5). Tumor burden at different time points within a group were compared with Student's t test (two-tailed). Kaplan-Meier survival analysis was performed to determine if endpoint curves differed. Statistical significance of endpoint curves was assessed with Mantel-Cox and Gehan-Bresholw-Wilcoxon Tests (GraphPad Prism version 5).

Western Blot Analysis of Keratinocyte Differentiation Markers.

To determine whether topical camphor treatment promotes keratinocyte differentiation in vivo, a separate cohort of adult female FVB mice were treated twice daily for a period of 5 days with increasing concentrations of topical camphor (0, 15%, 20% or 30% (wt/wt) camphor in acetone; N=3-4 mice per group). One day after the final topical dose, mice were sacrificed and epidermal lysates were prepared for immunoblot analysis according to standard protocols (Owens et al., J. Invest. Dermatol. 1996). Epidermal lysates were subjected to western blot analysis to measure levels of keratin 10 (K10, a keratinocyte terminal differentiation marker) and beta-tubulin (a house keeping gene used to normalize samples for protein loading). Protein levels were detected by peroxidase activated luminol exposure to X-ray film and estimated by densitometry using NIH-Image J software. Levels of K10 were normalized to levels of beta-tubulin for each sample; therefore, data are expressed as the ratio of K10 to beta-tubulin (K10/beta-tubulin). Mean protein levels between experimental groups were compared with Student's t tests (one-way, unpaired).

### Example 6

### Experiments In Vivo with a Mouse Model of SCC

In order to test camphor oil's effect on SCC tumor burden, a two-stage chemical-induced carcinogeneis mouse model (DMBA-TPA) (FIG. 10) was used to induce benign precancerous lesions and SCCS in adult female mice.

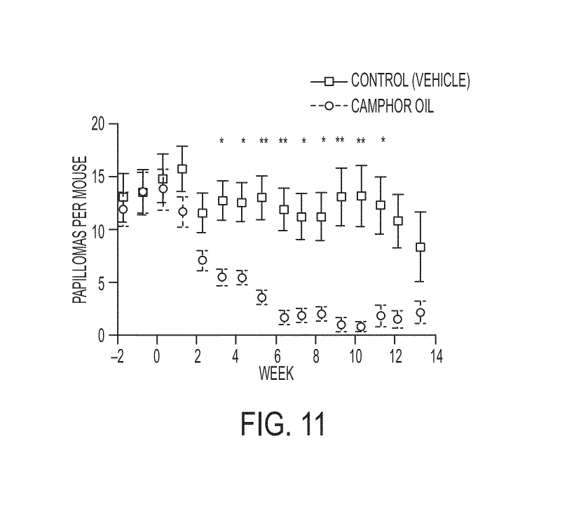

Daily topical treatment with 20% (wt/wt)camphor oil in acetone wt/wt was well tolerated by mice, although hyperkeratosis and slowed hair growth were observed in this group compared with vehicle-treated control mice. The effects on camphor oil on hair growth and hyperkeratosis are consistent with TRPV3 activation, as mutations that cause constitutive TRPV3 activation lead to hyperkeratotic lesions in humans (Olmsted syndrome; Danso-Abeam et al, 2013, PMID: 23692804) and hairlessness in mice (Xaio et al, 2008 PMID: 17706768). Camphor oil treatment caused a dramatic reduction in tumor burden in mice compared with vehicle-treated control mice (N=12 mice per group). This reduction was observed in all three measures of tumor burden that were assessed (FIGS. 11-16).

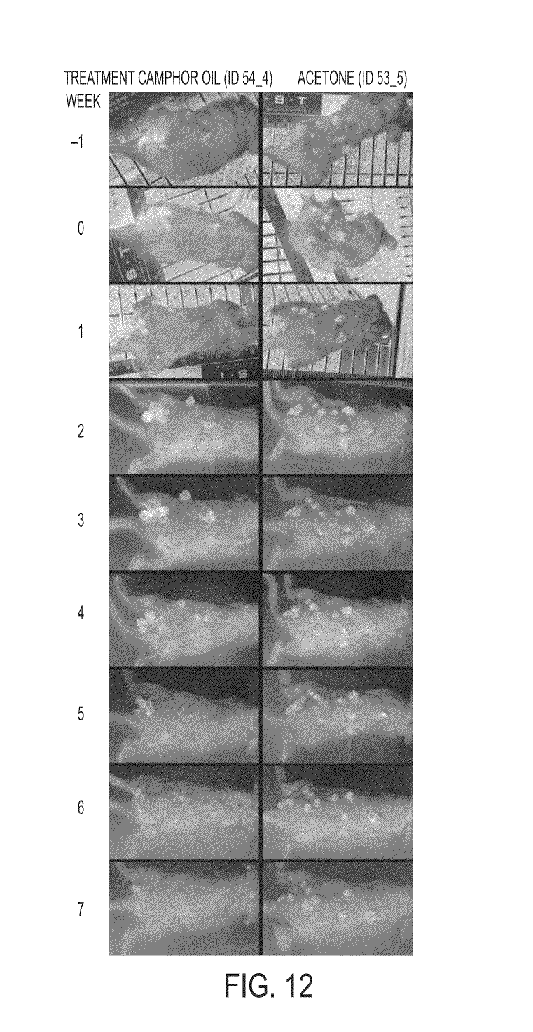

Mean Number of Pre-Malignant Tumors (FIGS. 11-12).

Both treatment groups were matched for the mean tumor burden prior to treatment (FIG. 11). Five days before treatment, mice assigned to the camphor-oil group displayed 13.5±2.0 tumors per mouse (mean±SEM; N=12) and those assigned to the control group had 13.5±2.2 tumors per mouse (N=12). The mean number of precancerous leisions per mouse did not differ significantly over time in the control group (week 13: 8.3±3.3 tumors per mouse, P=0.29; Student's t test, two-tailed). By contrast, the mean number of precancerous leisions per mouse decreased six-fold in the camphor-oil group (week 13: 2.2±1.1 tumors per mouse, P<0.001; Student's t test, two-tailed). A two-factor ANOVA showed a highly significant effect of treatment group on tumor persistence, with camphor-oil treated mice displaying fewer precancerous leisions [F(15,265)=101.40, P<0.0001]. The effect of treatment week was also highly significant [F(15,265)=5.16, P<0.0001]. Finally, there was a significant interaction effect between treatment group and treatment duration [F(15,265)=2.30, P=0.004]. Strikingly, post hoc analysis demonstrated that the tumor burden was significantly reduced in camphor-oil treated mice compared with control mice after only three weeks of once-daily treatment (FIG. 11). Reduction in tumor burden is illustrated in a photomontage of a representative mouse from each treatment group over seven weeks of treatment (FIG. 12). Thus, it was demonstrated that topical camphor oil treatment promotes regression of pre-malignant skin tumors.

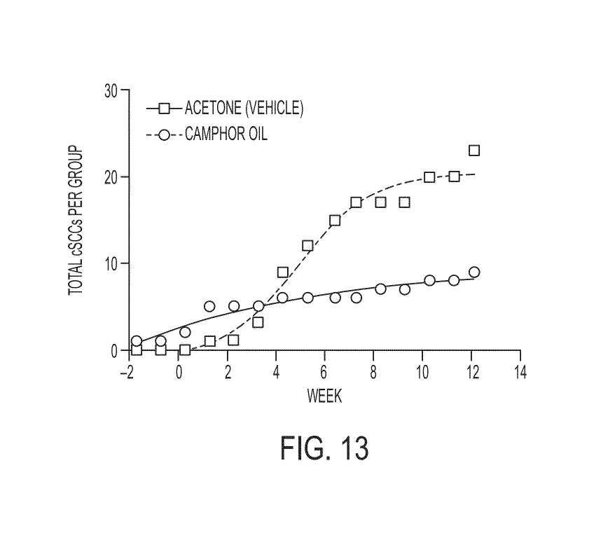

Number of Malignant SCCs (FIGS. 13-15).

Mice treated with camphor oil for 13 weeks developed 2.5-fold fewer SCCs than vehicle-treated control mice (camphor-oil group: 9 SCCs; control group: 23 SCCs; FIG. 13). Note that one mouse randomly assigned to the camphor-oil group developed a malignant SCC before camphor oil-treatment began. A two-factor ANOVA indicated a highly significant effect of treatment group on the number of malignant SCCs [F(1,15)=10.20, P=0.006]. The effect of treatment time was also significant [F(15,15)=2.74, P=0.030]. Thus, this analysis indicates that camphor oil slows the progression of benign tumors to SCC.

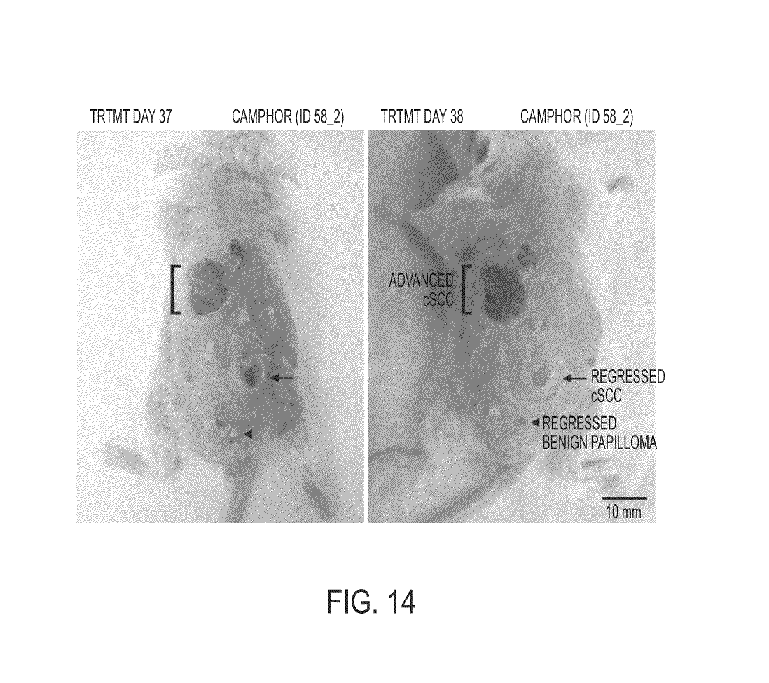

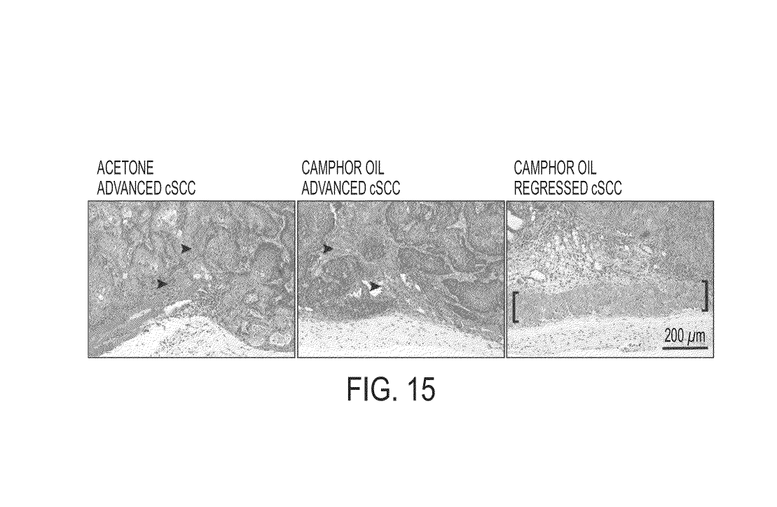

Along with developing fewer SCCs, mice treated with camphor oil displayed apparent regressions of a subset of early-stage SCCs (FIG. 14). Such regressions were never observed in vehicle-treated control mice. When advanced SCCs developed, they progressed to experimental endpoints in both treatment groups. Histopathological examination of regressed SCCs revealed microscopic areas of residual tumor that resembled SCC in situ. Residual lesions in camphor oil-treated mice displayed intact fascia and muscle layers (FIG. 15, right panel). By contrast, advanced SCCs invaded the fascia and muscle layers (FIG. 15 left and middle panels). These results indicate that daily camphor-oil treatment can suppress SCC proliferation and invasion although it may not completely eradicate SCC lesions. These data indicate that camphor-oil treatment dramatically attenuates malignant SCC conversion in this carcinogenesis model.

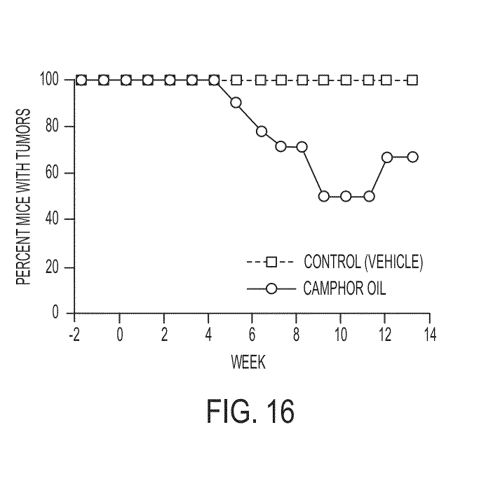

Tumor incidence (FIG. 16).

At the end of the 15-week TPA treatment period, 100% of mice in both groups had tumors. Tumor incidence remained at 100% in control mice but decreased to as low as 50% in camphor oil-treated mice (FIG. 16). A two-factor ANOVA showed a highly significant effect of treatment group on tumor incidence [F(1,15)=14.40, P=0.0018]. The effect of treatment day was not significant [F(15,15)=1.0, NS]. Therefore, camphor-oil treatment is sufficient to completely clear tumors in a subset of animals in this carcinogenesis model.

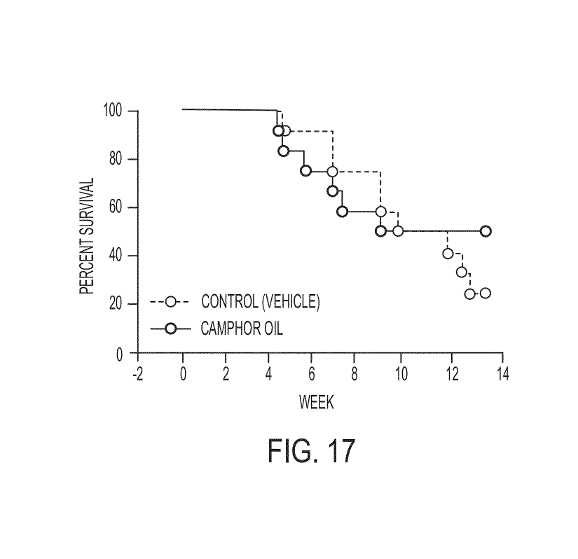

Endpoint Curves (FIG. 17).

After 13 weeks of treatment, 75% of control mice and 50% of camphor oil-treated mice had reached an experimental endpoint; however, Kaplan-Meier survival analysis indicated that endpoint curves did not differ significantly between treatment groups (P>0.53, Mantel-Cox and Gehan-Bresholw-Wilcoxon tests; FIG. 17). Specific allowances for a survival endpoint were not incorporated into initial experimental design. The effects of camphor oil were tested on the progression of pre-established skin tumors. Relatively high doses of DMBA and TPA were used to increase overall skin tumor burden and the rate of malignant conversion (SCC formation). This approach was utilized in order to generate a large number pre-malignant tumor targets for camphor treatment. This increased the statistical power of any anti-tumor effects observed following camphor treatment. The utility of this approach is appreciated in FIGS. 11-16. Animal welfare guidelines require that animals be euthanized based on the endpoints of any single tumor and do not discriminate between animals with multiple malignant SCCs. Although control mice were routinely euthanized with multiple SCC lesions, camphor mice exhibited a marked reduction in SCC formation (FIG. 13) yet were required to be euthanized along a similar time frame due to the endpoints of a single SCC as opposed to multiple SCCs observed in control mice. Camphor oil treatment would dramatically increase survival under an experimental design where the overall tumor burden was lower (e.g., when control mice sustain a maximum of 1-2 SCCs). Overall, camphor oil treatment is highly effective at reducing the rate of malignant conversion of benign epidermal lesions to SCCs in vivo. Strikingly, camphor oil treatment appears to lead to dramatic regression of pre-established neoplastic skin lesions and these changes in tumor regression could be observed on a daily basis (FIGS. 12 & 14). These findings are consistent with in vitro results described herein, which show that camphor blocks proliferation and that 2-APB induces the expression of markers of terminal differentiation in cultured human keratinocytes. Therefore, the anti-tumor effects of camphor may be due to its ability to block neoplastic proliferation in vivo and shift transformed cells to commit to terminal differentiation.

### Example 7

### Topical Camphor Treatment Promotes Keratinocyte Differentiation In Vivo

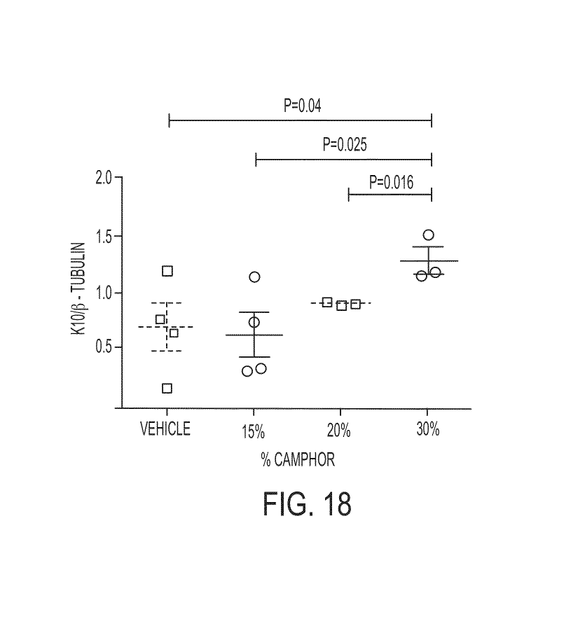

Western Blot Analysis (FIG. 18).

To determine whether topical camphor treatment promotes keratinocyte differentiation in vivo, adult female FVB mice (n=3-4 per group) were treated twice daily for a period of 5 days with increasing concentrations of topical camphor (15%, 20% or 30% (wt/wt)) or vehicle only. Whole cell epidermal lysates were generated from each group and subjected to western blot analysis to detect the levels of the terminal differentiation marker cytokeratin, Keratin 10 (K10). Expression of K10, a keratinocyte terminal differentiation marker, increased with camphor treatment in a dose-dependent manner (FIG. 18). Mice treated with 30% (wt/wt) camphor displayed significantly higher K10 protein levels compared with mice treated with lower camphor concentrations or vehicle alone (P=0.04; Student's t test). It was determined that topical camphor treatment upregulates levels of a keratinocyte terminal differentiation marker in vivo. These data confirm and extend in vitro studies of human keratinocytes as described herein.

### Example 8

### Testing Camphor Oil Constituents for Antitumor Activity and Regulation of Keratinocyte Physiology

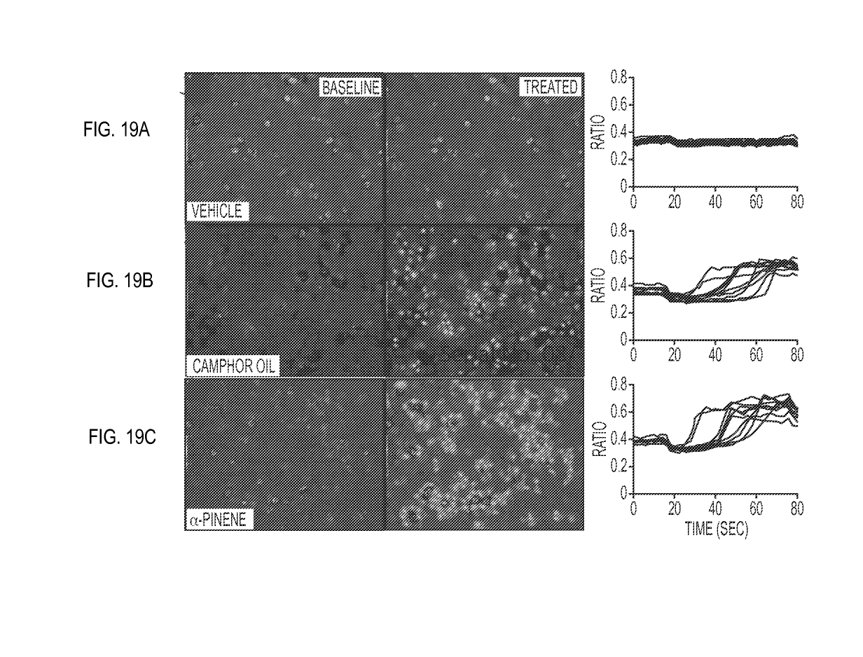

In keratinocytes, increased intracellular calcium stimulates the terminal differentiation process. To determine whether camphor oil and its abundant constituents are directly bioactive on epithelial keratinocytes, a ratiometric Ca2+ indictor (Fura-2) was employed to test whether a panel of terpenes elicited cytoplasmic Ca2+ increases in undifferentiated human epidermal keratinocytes (FIG. 19A-19C). Keratinocytes were cultured according to our standard methods. For live-cell imaging, keratinocytes were bathed in Ringer's solution containing 2 mM Ca2+. Within 1 minute of perfusion, both camphor oil and α-pinene stimulated detectable calcium signals in a subset of undifferentiated human keratinocytes, whereas eucalyptol showed no effects at the concentration tested and under these experimental conditions; however, other concentrations may be effective or it may have synergy with other active agents. Certain embodiments are directed to combinations of one or more active agents. compared with vehicle-treated control cells. Thus, camphor oil directly stimulates Ca2+ influx in human keratinocytes, and that this effect can be recapitulated by some but not all constituents.

The terpenes in camphor oil, as well as other plant essential oils, have not been studied well in vivo for their effectiveness in reducing the growth of tumor cells. The potential effectiveness of camphor oil constituents on tumor cell growth makes skin tumor progression a useful experimental platform to uncover the biological basis of camphor oil activity. Whether camphor oil contains bioactive compounds that slow cSCC growth in vivo was tested.

Methods for analyzing mouse skin in vivo and human keratinocytes in vitro are well established in the art and may be used by the person of skill in the art. Adult female FVB/N mice (7-52 weeks old) were used here because of the high conversion rate of premalignant tumors to malignant SCCs in this strain. Based on the data, ≦15 mice per group are sufficient for these tests. Statistical significance can be assessed by Student's t tests (unpaired, two-tailed) or ANOVA (one-way for multiple groups; two-way followed by Bonferroni post hoc for longitudinal studies) and Kaplan-Meier survival analysis.

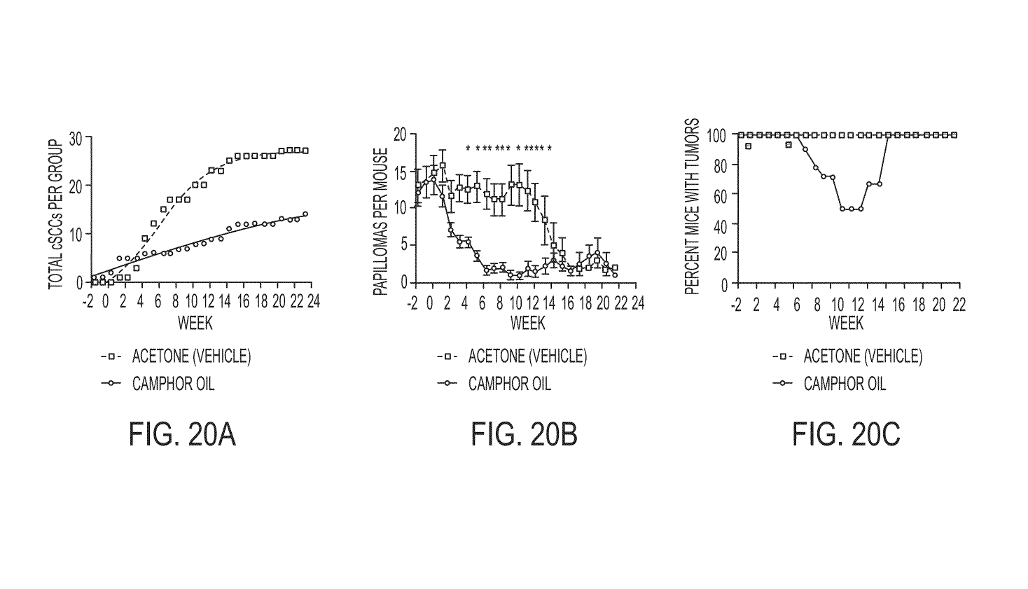

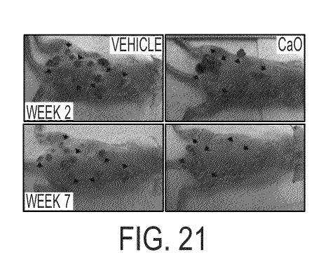

Keratinocyte-derived tumors were induced in mice using a two-step chemical carcinogenesis model (DMBA/TPA (FIG. 10, FIG. 20A-20C). After 15 weeks of TPA treatment, which is the endpoint of the study, mice were administered daily, topical applications of camphor oil (20% (wt/wt) in acetone) or vehicle for 24 weeks (N=12 mice per group). Tumor burden (number of pre-malignant lesions and cSCCs) was assessed weekly. At endpoint, all tumors were collected and tumor grades (pre-malignant lesion, invasive cSCC and secondary metastasis) were confirmed based on histological criteria. Preliminary studies indicated that camphor oil has potent antitumor capacity in vivo (FIG. 21). Camphor oil treatment dramatically slowed malignant tumor progression, resulting in half as many cSCCs overall (FIG. 20A).

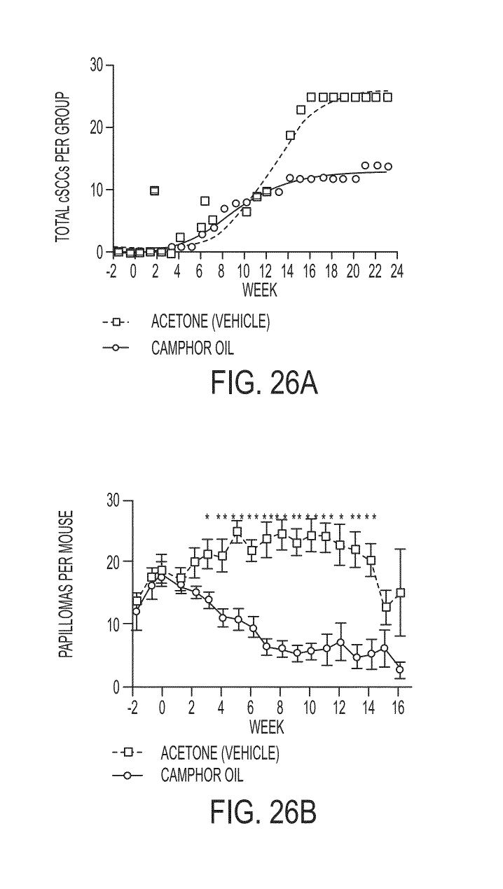

Camphor oil also caused regression of pre-malignant tumors and reduced tumor incidence in vivo (FIG. 2B-2C). See also FIG. 21; arrows map tumor persistence or loss). These effects were evident within only four weeks of camphor oil treatment. An independent cohort replicated these results (N=10 mice per group. Data not shown). To ensure sufficient power to observe subtle effects on tumor progression in these studies, an aggressive carcinogenesis protocol was used to achieve multiple lesions per mouse (mean±SD: 14.3±7.2, N=24 mice). Thus, almost all mice ultimately developed ≧1 malignant cSCC. This suggests that a subset of skin tumors advance to a point at which camphor oil cannot halt all progression at the time of treatment. Nonetheless, the high percentage of responsive tumors and dramatically lower cSCC burden (FIG. 20A-20C) suggests that earlier treatment times may increase the number of responding tumors. A second cohort replicated both the regression of precancerous leisions and the reduced incidence of cSCC tumors [N=11 mice per group, Two-way ANOVA; P<0.0001 for both comparisons. See FIG. 26A-26B. Together, these results demonstrate camphor oil's striking chemopreventive effects on cSCC progression in vivo.

Collectively, these findings provide strong support by showing that camphor oil is bioactive in epithelial keratinocytes, stimulates calcium signaling and suppresses malignant keratinocyte growth in vivo. In addition, we have identified the major terpenoid constituents of camphor oil. See also Example 12.

### Example 9

### Two-Step Carcinogenesis Model

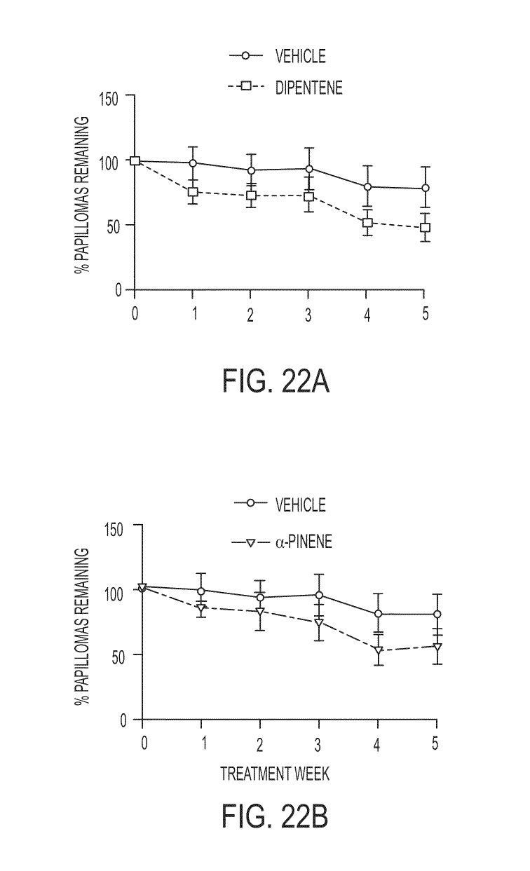

Because the composition of camphor oil varies among lots, the biological effects of camphor oil and its constituents on skin homeostasis were tested on healthy and neoplastic keratinocytes. To test whether compounds in camphor oil have antitumor effects, a two-step chemical carcinogenesis model was used (FIG. 10). After tumor induction, mice were treated with the four most abundant terpene constituents for five weeks. This time point was chosen because pre-malignant tumor burden is reduced by >50% after five weeks of camphor oil treatment. Each mouse received a topical application once daily of vehicle or an individual terpene (20% wt/wt dipentene, eucalyptol, α-pinene, or γ-terpinene in acetone). Two components of camphor oil, α-pinene and dipentene, showed antitumor effects in vivo (FIG. 22A-22B). The results show that both agents significantly reduced the % of precancerous lesion s per mouse steadily over the 5 week treatment period. The effects of dipentene and α-pinene are similar to those of camphor oil, suggesting that these compounds might contribute to the anti-tumor effects of camphor oil.

### Example 10

### Dose-Response Relationships for Camphor White Oil Treatment

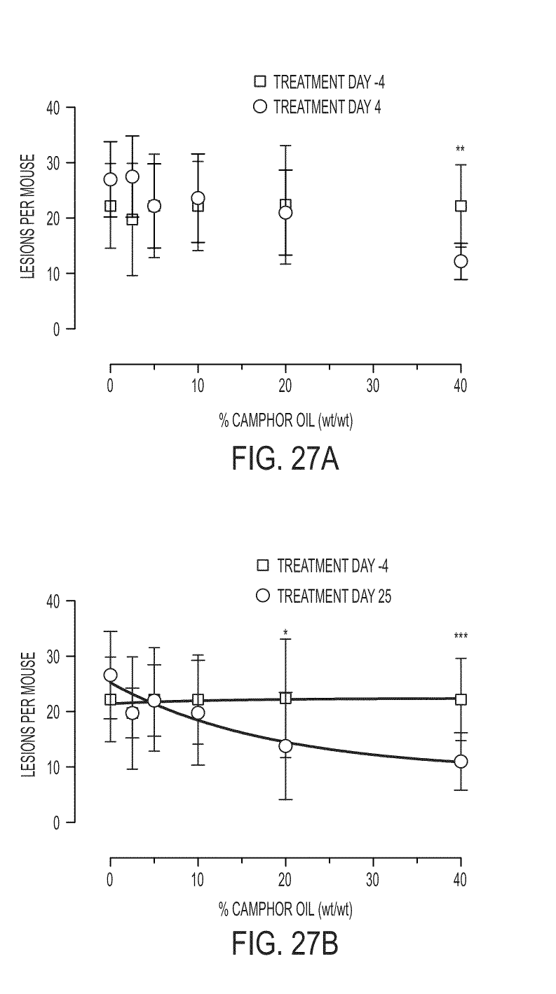

Dose response studies were performed to determine the optimal dose of camphor white oil in treating skin tumors in mice. Tumors were induced as in cohort one (DMBA start date 1.3.2015, TPA 13 weeks Jan, 16, 2015 through Apr. 14, 2015). Mice were distributed to achieve the same number of tumors in each group and then randomly assigned to a treatment group (0%, 2.5%, 5%, 10%, 20%, or 40% camphor oil). Mice were treated once daily with acetone vehicle (0%) or camphor oil in varying concentrations and tumors were counted once per week. We identified a significant reduction in the number of premalignant tumors at 4 days of treatment with 40% camphor oil. In addition, we found a significant decrease in tumors at 25 days of treatment with 20% and 40% camphor oil, whereas no significant differences were found with treatment at lower concentrations (FIG. 27A-27B). These data suggest that camphor oil applied between 20-40% can alleviate skin tumors in mice.

### Example 11

### Effects of Camphor Oil Constituents on Keratinocyte Proliferation

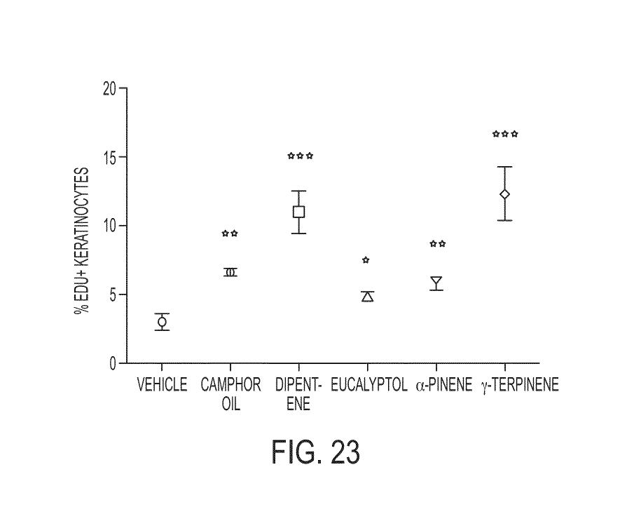

Age matched FVB (female mice purchased from Jackson labs) aged 6-8 weeks were shave on their dorsal sides. Mice were then treated for five consecutive days with vehicle, 20% camphor white oil, 20% eucalyptol, 20% α-pinene, 20% dipentene, or 20% γ-terpinene (2-6 mice per group). Mice were then injected with EdU and sacrificed one hour later. The epidermis was isolated and stained for Itgα6, Sca1+, and EDU. Itgα6+Sca1+EdU+ cells were then identified on a flow cytometer. All of these compounds caused significant increases in keratinocyte proliferation compared with vehicle (t-test eucalyptol p<0.05, camphor oil and α-pinene P<0.01, dipentene and γ-terpinene P<0.001) (FIG. 23). Without being bound by theory, this result might reflect distinct effects of terpenes in normal (proliferation) versus tumor (regression) keratinocytes. Alternatively, increased proliferation could be balanced by increased apoptosis or differentiation in skin tumors.

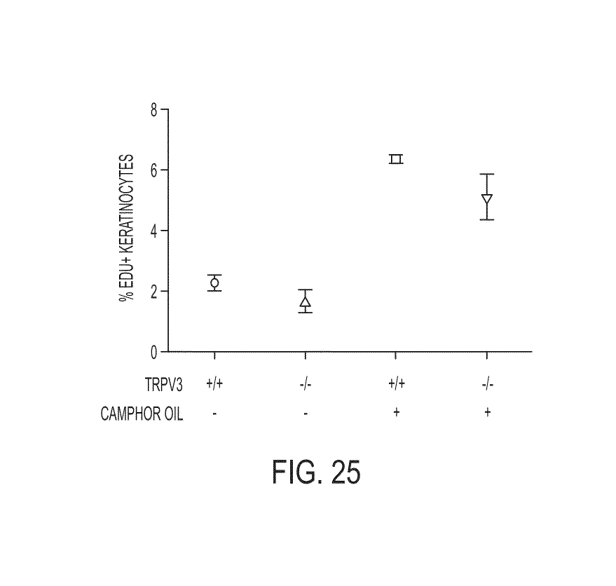

To test whether effects of camphor white oil on normal keratinocytes are TrpV3 dependent proliferation in TrpV3 mutant and wild type mice treated with camphor oil was analyzed. TrpV3 mutant and wild type mice aged 6-8 weeks were shaved on their dorsal sides. Mice were then treated for five consecutive days with vehicle or 20% camphor oil (4-5 mice per group). Mice were then injected with EdU (5-ethynyl-2′-deoxyuridine) and sacrificed one hour later. The epidermis was isolated and stained for Itga6, Sca1, and EdU. Itga6+Sca1+EdU+ cells were then identified on a flow cytometer to identify cells in the intrafollicular epidermis that are actively proliferating. TrpV3 mutation was found to have no significant effect on camphor oil evoked proliferation, indicating that proliferation in normal kertinocytes is due to a non-TrpV3 dependent mechanism (FIG. 25)

### Example 12

### Effects of Camphor White Oil on Precancerous Lesions and SCC

Camphor white oil was tested in vivo for anti-tumor activity using a two-step chemical carcinogenesis model of cSCC in mice. Precancerous leisions and cSCCs were initiated by DMBA followed by application of TPA twice weekly for 12 weeks. After precancerous leisions had developed, mice were randomly assigned to two treatment groups (N=12 mice per group; extract treated: 13.5±6.8 lesions per mouse (mean±SD); vehicle control: 13.5±7.5 lesions per mouse; P=NS, Student's two-tailed t test). Mice were then treated daily with topical camphor white oil in acetone vehicle for up to 29 weeks. precancerous leisions and cSCCs were quantified weekly, and mice were euthanized when they developed at least one cSCC of diameter ≧2 cm. Treatment with extracts caused a dramatic regression of pre-malignant tumors compared with vehicle controls [Two-way ANOVA; P<0.0001; group effect F(1,284)=30.98] (FIG. 20A-20C). For example, five weeks after treatment onset, the number of precancerous leisions per mouse decreased by 60% in the extract-treated group but was unchanged in the vehicle control group (extract treated: 3.6±2.2 lesions per mouse; vehicle control: 13±6.8 lesions per mouse; P<0.01, Bonferroni post hoc correction for multiple comparisons). Importantly, skin tumor treatment was associated with an overall two-fold decrease in the incidence of malignant cSCC tumors [Two-way ANOVA; P<0.0001; group effect F(1,25)=43.2]. A second cohort replicated both the regression of precancerous leisions and the reduced incidence of cSCC tumors [N=11 mice per group, Two-way ANOVA; P<0.0001 for both comparisons] (FIG. 26A-26B).

In the present specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. The contents of all references, pending patent applications and published patents, cited throughout this application are hereby expressly incorporated by reference as if set forth herein in their entirety, except where terminology is not consistent with the definitions herein. Although specific terms are employed, they are used as in the art unless otherwise indicated.

### REFERENCES

All citations (e.g., scientific journal publications, patents, and other reference material) mentioned herein are hereby incorporated herein by reference to the same extent as if each individual citation was specifically and individually indicated to be incorporated by reference.
1. Bachelor M A, Lu Y, Owens D M (2011) L-3-Phosphoserine phosphatase (PSPH) regulates cutaneous squamous cell carcinoma proliferation independent of L-serine biosynthesis. Journal of dermatological science 63: 164-172. 3152677 3152677
2. Bauman J E, Michel L S, Chung C H (2012) New promising molecular targets in head and neck squamous cell carcinoma. Current opinion in oncology 24: 235-242.
3. Bautista D M, Jordt S E, Nikai T, Tsuruda P R, Read A J, Poblete J, Yamoah E N, Basbaum A I, Julius D (2006) TRPA1 mediates the inflammatory actions of environmental irritants and proalgesic agents. Cell 124: 1269-1282.
4. Beck B, Lehen'kyi V, Roudbaraki M, Flourakis M, Charveron M, Bordat P, Polakowska R, Prevarskaya N, Skryma R (2008) TRPC channels determine human keratinocyte differentiation: new insight into basal cell carcinoma. Cell calcium 43: 492-505.
5. Bode A M, Cho Y Y, Zheng D, Zhu F, Ericson M E, Ma W Y, Yao K, Dong Z (2009) Transient receptor potential type vanilloid 1 suppresses skin carcinogenesis. Cancer research 69: 905-913. 2669313 2669313
6. Borbiro I, Lisztes E, Toth B I, Czifra G, Olah A, Szollosi A G, Szentandrassy N, Nanasi P P, Peter Z, Paus R, Kovacs L, Biro T (2011) Activation of transient receptor potential vanilloid-3 inhibitshuman hair growth. The Journal of investigative dermatology 131: 1605-1614.
7. Cai S, Fatherazi S, Presland R B, Belton C M, Roberts F A, Goodwin P C, Schubert M M, Izutsu K T (2006) Evidence that TRPC1 contributes to calcium-induced differentiation of human keratinocytes. Pflugers Arch 452: 43-52.
8. Cheng X, Jin J, Hu L, Shen D, Dong X P, Samie M A, Knoff J, Eisinger B, Liu M L, Huang S M, Caterina M J, Dempsey P, Michael L E, Dlugosz A A, Andrews N C, Clapham D E, Xu H (2010) TRP channel regulates EGFR signaling in hair morphogenesis and skin barrier formation. Cell 141: 331-343. 2858065 2858065
9. Chung M K, Lee H, Mizuno A, Suzuki M, Caterina M J (2004a) 2-aminoethoxydiphenyl borate activates and sensitizes the heat-gated ion channel TRPV3. The Journal of neuroscience: the official journal of the Society for Neuroscience 24: 5177-5182.
10. Chung M K, Lee H, Mizuno A, Suzuki M, Caterina M J (2004b) TRPV3 and TRPV4 mediate warmth-evoked currents in primary mouse keratinocytes. The Journal of biological chemistry 279: 21569-21575.
11. Commandeur S, de Gruijl F R, Willemze R, Tensen C P, El Ghalbzouri A (2009) An in vitro three-dimensional model of primary human cutaneous squamous cell carcinoma. Experimental dermatology 18: 849-856.
12. Denda M, Tsutsumi M (2011) Roles of transient receptor potential proteins (TRPs) in epidermal keratinocytes. Adv Exp Med Biol 704: 847-860.
13. A, Kalwa H, Storch U, Mederos y Schnitzler M, Salanova B, Pinkenburg O, Dubrovska G, Essin K, Gollasch M, Birnbaumer L, Gudermann T (2007) Pressure-induced and store-operated cation influx in vascular smooth muscle cells is independent of TRPC1. Pflugers Arch 455: 465-477.
14. Gavva N R, Tamir R, Qu Y, Klionsky L, Zhang T J, Immke D, Wang J, Zhu D, Vanderah T W, Porreca F, Doherty E M, Norman M H, Wild K D, Bannon A W, Louis J C, Treanor J J (2005) AMG 9810 [(E)-3-(4-t-butylphenyl)-N-(2,3-dihydrobenzo[b][1,4]dioxin-6-yl)acrylamide], a novel vanilloid receptor 1 (TRPV1) antagonist with antihyperalgesic properties. J Pharmacol Exp Ther 313: 474-484.
15. Hardisson D (2003) Molecular pathogenesis of head and neck squamous cell carcinoma. European archives of oto-rhino-laryngology: official journal of the European Federation of Oto-Rhino-Laryngological Societies 260: 502-508.
16. He B, Liu F, Ruan J, Li A, Chen J, Li R, Shen J, Zheng D, Luo R (2012) Silencing TRPC1 expression inhibits invasion of CNE2 nasopharyngeal tumor cells. Oncology reports 27: 1548-1554.
17. Hennings H, Michael D, Cheng C, Steinert P, Holbrook K, Yuspa S H (1980) Calcium regulation of growth and differentiation of mouse epidermal cells in culture. Cell 19: 245-254.
18. Hu H, Grandl J, Bandell M, Petrus M, Patapoutian A (2009) Two amino acid residues determine 2-APB sensitivity of the ion channels TRPV3 and TRPV4. Proceedings of the National Academy of Sciences of the United States of America 106: 1626-1631. 2635798 2635798
19. Kida N, Sokabe T, Kashio M, Haruna K, Mizuno Y, Suga Y, Nishikawa K, Kanamaru A, Hongo M, Oba A, Tominaga M (2012) Importance of transient receptor potential vanilloid 4 (TRPV4) in epidermal barrier function in human skin keratinocytes. Pflugers Archiv: European journal of physiology 463: 715-725.
20. Kim Y S, Bahn K N, Hah C K, Gang H I, Ha Y L (2008) Inhibition of 7,12-dimethylbenz[a]anthracene induced mouse skin carcinogenesis by Artemisia capillaris. Journal of food science 73: T16-20.
21. Leuner K, Kraus M, Woelfle U, Beschmann H, Harteneck C, Boehncke W H, Schempp C M, Muller W E (2011) Reduced TRPC channel expression in psoriatic keratinocytes is associated with impaired differentiation and enhanced proliferation. PloS one 6: e14716. 3043053 3043053
22. Li L, Tucker R W, Hennings H, Yuspa S H (1995) Chelation of intracellular calcium inhibits murine keratinocyte differentiation in vitro. Journal of cellular physiology 163: 105-114.
23. Lin Z, Chen Q, Lee M, Cao X, Zhang J, Ma D, Chen L, Hu X, Wang H, Wang X, Zhang P, Liu X, Guan L, Tang Y, Yang H, Tu P, Bu D, Zhu X, Wang K, Li R, Yang Y (2012) Exome sequencing reveals mutations in TRPV3 as a cause of Olmsted syndrome. American journal of human genetics 90: 558-564. 3309189 3309189
24. Mascia F, Denning M, Kopan R, Yuspa S H (2012) The black box illuminated: signals and signaling. The Journal of investigative dermatology 132: 811-819.
25. Moqrich A, Hwang S W, Earley T J, Petrus M J, Murray A N, Spencer K S, Andahazy M, Story G M, Patapoutian A (2005) Impaired thermosensation in mice lacking TRPV3, a heat and camphor sensor in the skin. Science 307: 1468-1472.
26. Obrigkeit D H, Jugert F K, Beermann T, Baron J M, Frank J, Merk H F, Bickers D R, Abuzahra F (2009) Effects of photodynamic therapy evaluated in a novel three-dimensional squamous cell carcinoma organ construct of the skin. Photochemistry and photobiology 85: 272-278.
27. Okamoto Y, Ohkubo T, Ikebe T, Yamazaki J (2012) Blockade of TRPM8 activity reduces the invasion potential of oral squamous carcinoma cell lines. International journal of oncology 40: 1431-1440.
28. Pan Z, Wang Z, Yang H, Zhang F, Reinach P S (2011) TRPV1 activation is required for hypertonicity-stimulated inflammatory cytokine release in human corneal epithelial cells. Invest Ophthalmol Vis Sci 52: 485-493. 3053292 3053292
29. Pogatzki-Zahn E M, Shimizu I, Caterina M, Raja S N (2005) Heat hyperalgesia after incision requires TRPV1 and is distinct from pure inflammatory pain. Pain 115: 296-307.
30. Prevarskaya N, Zhang L, Barritt G (2007) TRP channels in cancer. Biochimica et biophysica acta 1772: 937-946.
31. Ratushny V, Gober M D, Hick R, Ridky T W, Seykora J T (2012) From keratinocyte to cancer: the pathogenesis and modeling of cutaneous squamous cell carcinoma. The Journal of clinical investigation 122: 464-472. 3266779 3266779
32. Santoni G, Caprodossi S, Farfariello V, Liberati S, Gismondi A, Amantini C (2012) Antioncogenic effects of transient receptor potential vanilloid 1 in the progression of transitional urothelial cancer of human bladder. ISRN urology 2012: 458238. 3302024 3302024
33. Sawada Y, Hosokawa H, Hori A, Matsumura K, Kobayashi S (2007) Cold sensitivity of recombinant TRPA1 channels. Brain Res 1160: 39-46.
34. Schneider M R, Werner S, Paus R, Wolf E (2008) Beyond wavy hairs: the epidermal growth factor receptor and its ligands in skin biology and pathology. The American journal of pathology 173: 14-24. 2438281 2438281
35. Sibilia M, Fleischmann A, Behrens A, Stingl L, Carroll J, Watt F M, Schlessinger J, Wagner E F (2000) The EGF receptor provides an essential survival signal for SOS-dependent skin tumor development. Cell 102: 211-220.
36. Tajeddine N, Gailly P (2012) TRPC1 protein channel is major regulator of epidermal growth factor receptor signaling. The Journal of biological chemistry 287: 16146-16157. 3351290 3351290
37. Vanden Abeele F, Shuba Y, Roudbaraki M, Lemonnier L, Vanoverberghe K, Mariot P, Skryma R, Prevarskaya N (2003) Store-operated calcium channels in prostate cancer epithelial cells: function, regulation, and role in carcinogenesis. Cell calcium 33: 357-373.
38. Xu H, Blair N T, Clapham D E (2005) Camphor activates and strongly desensitizes the transient receptor potential vanilloid subtype 1 channel in a vanilloid-independent mechanism. J Neurosc 25: 8924-8937
39. Hennings, H., Michael, D., Cheng, C., Steinert, P., Holbrook, K., and Yuspa, S. H. 1980. Calcium regulation of growth and differentiation of mouse epidermal cells in culture. Cell 19:245-254.
40. Obrigkeit, D. H., Jugert, F. K., Beermann, T., Baron, J. M., Frank, J., Merk, H. F., Bickers, D. R., and Abuzahra, F. 2009. Effects of photodynamic therapy evaluated in a novel three-dimensional squamous cell carcinoma organ construct of the skin. Photochem Photobiol 85:272-278.
41. Commandeur, S., de Gruijl, F. R., Willemze, R., Tensen, C. P., and El Ghalbzouri, A. 2009. An in vitro three-dimensional model of primary human cutaneous squamous cell carcinoma. Exp Dermatol 18:849-856.
42. Bachelor, M. A., Lu, Y., and Owens, D. M. 2011. L-3-Phosphoserine phosphatase (PSPH) regulates cutaneous squamous cell carcinoma proliferation independent of L-serine biosynthesis. J Dermatol Sci 63:164-172.
43. Prevarskaya, N., Zhang, L., and Barritt, G. 2007. TRP channels in cancer. Biochim Biophys Acta 1772:937-946.
44. Bode, A. M., Cho, Y. Y., Zheng, D., Zhu, F., Ericson, M. E., Ma, W. Y., Yao, K., and Dong, Z. 2009. Transient receptor potential type vanilloid 1 suppresses skin carcinogenesis. Cancer Res 69:905-913.
45. He, B., Liu, F., Ruan, J., Li, A., Chen, J., Li, R., Shen, J., Zheng, D., and Luo, R. 2012. Silencing TRPC1 expression inhibits invasion of CNE2 nasopharyngeal tumor cells. Oncol Rep 27:1548-1554.
46. Okamoto, Y., Ohkubo, T., Ikebe, T., and Yamazaki, J. 2012. Blockade of TRPM8 activity reduces the invasion potential of oral squamous carcinoma cell lines. Int J Oncol 40:1431-1440.
47. Santoni, G., Caprodossi, S., Farfariello, V., Liberati, S., Gismondi, A., and Amantini, C. 2012. Antioncogenic effects of transient receptor potential vanilloid 1 in the progression of transitional urothelial cancer of human bladder. ISRN Urol 2012:458238.
48. Kim, Y. S., Bahn, K. N., Hah, C. K., Gang, H. I., and Ha, Y. L. 2008. Inhibition of 7,12-dimethylbenz[a]anthracene induced mouse skin carcinogenesis by Artemisia capillaris. J Food Sci 73:T16-20.
49. Sibilia, M., Fleischmann, A., Behrens, A., Stingl, L., Carroll, J., Watt, F. M., Schlessinger, J., and Wagner, E. F. 2000. The EGF receptor provides an essential survival signal for SOS-dependent skin tumor development. Cell 102:211-220.
50. Vogt-Eisele, A K. et al, Monoterpenoid agonists of TRPV3, Br J Pharmacol 2007 151:530-540; (Apr. 10, 2007).
51. Danso-Abeam, D. et al, Olmsted Syndrome: exploration of the immunological phenotype. Orphanet J Rare Dis. 2013, May 21; 8: 79, PMID: 23692804.
52. Xaio R., et al, The TRPV3 mutation associated with the hairless phenotype in rodens is constitutively active., Cell Calcium, 2008 April; 43 (4): 334-43. PMID: 17706768).
53. Yang, F. et al. Linalool, derived from Cinnamomum camphora (L.) Presl leaf extracts, possesses molluscicidal activity against Oncomelania hupensis and inhibits infection of Schistosoma japonicum. Parasit Vectors 7, 407, doi:10.1186/1756-3305-7-407 [pii] (2014).
54. Lee, H. J. et al. In vitro anti-inflammatory and anti-oxidative effects of Cinnamomum camphora extracts. J Ethnopharmacol 103, 208-216, doi:S0378-8741(05)00511-8 [pii] (2006).
55. Satyal, P. et al. Bioactivities and compositional analyses of Cinnamomum essential oils from Nepal: C. camphora, C. tamala, and C. glaucescens. Nat Prod Commun 8, 1777-1784 (2013).
56. Liu, C. H., Chen, C. Y., Huang, A. M. & Li, J. H. Subamolide A, a component isolated from Cinnamomum subavenium, induces apoptosis mediated by mitochondria-dependent, p53 and ERK1/2 pathways in human urothelial carcinoma cell line NTUB1. J Ethnopharmacol 137, 503-511, doi:10.1016/j.jep.2011.06.001 S0378-8741(11)00418-1 [pii] (2011).
57. Bayala, B. et al. Chemical composition, antioxidant, anti-inflammatory and anti-proliferative activities of essential oils of plants from burkina faso. PLoS One 9, e92122, doi:10.1371/journal.pone.0092122 PONE-D-13-52038 [pii] (2014).
58. Kusuhara, M. et al. Fragrant environment with alpha-pinene decreases tumor growth in mice. Biomed Res 33, 57-61, doi:JST.JSTAGE/biomedres/33.57 [pii] (2012).
59. Russin, W. A., Hoesly, J. D., Elson, C. E., Tanner, M. A. & Gould, M. N. Inhibition of rat mammary carcinogenesis by terpenoids. Carcinogenesis 10, 2161-2164 (1989).
60. Chidambara Murthy, K. N., Jayaprakasha, G. K. & Patil, B. S. D-limonene rich volatile oil from blood oranges inhibits angiogenesis, metastasis and cell death in human colon cancer cells. Life Sci 91, 429-439, doi:10.1016/j.lfs.2012.08.016 50024-3205(12)00444-4 [pii] (2012).
61. Bhattacharjee, B. & Chatterjee, J. Identification of proapoptopic, anti-inflammatory, anti-proliferative, anti-invasive and anti-angiogenic targets of essential oils in cardamom by dual reverse virtual screening and binding pose analysis. Asian Pac J Cancer Prev 14, 3735-3742 (2013).
62. Chaudhary, S. C., Siddiqui, M. S., Athar, M. & Alam, M. S. D-Limonene modulates inflammation, oxidative stress and Ras-ERK pathway to inhibit murine skin tumorigenesis. Hum Exp Toxicol 31, 798-811, doi:10.1177/0960327111434948 0960327111434948 [pii] (2012).
63. Salminen, A., Lehtonen, M., Suuronen, T., Kaarniranta, K. & Huuskonen, J. Terpenoids: natural inhibitors of NF-kappaB signaling with anti-inflammatory and anticancer potential. Cell Mol Life Sci 65, 2979-2999, doi:10.1007/s00018-008-8103-5 (2008).
64. Watt, F. M. Terminal differentiation of epidermal keratinocytes. Curr Opin Cell Biol 1, 1107-1115 (1989).
65. Haeberle, H. et al. Molecular profiling reveals synaptic release machinery in Merkel cells. Proc Natl Acad Sci USA 101, 14503-14508, doi:10.1073/pnas.0406308101 [pii] (2004).
66. Owens, D. M., Spalding, J. W., Tennant, R. W. & Smart, R. C. Genetic alterations cooperate with v-Ha-ras to accelerate multistage carcinogenesis in TG.AC transgenic mouse skin. Cancer Res 55, 3171-3178 (1995).
67. Thompson, E. A. et al. C/EBPalpha expression is downregulated in human nonmelanoma skin cancers and inactivation of C/EBPalpha confers susceptibility to UVB-induced skin squamous cell carcinomas. J Invest Dermatol 131, 1339-1346, doi:10.1038/jid.2011.31
68. Wilson, S. R. et al. The ion channel TRPA1 is required for chronic itch. J Neurosci 33, 9283-9294, doi:10.1523/JNEUROSCI.5318-12.2013 33/22/9283 [pii] (2013).
69. Owens, D. M., Romero, M. R., Gardner, C. & Watt, F. M. Suprabasal alpha6beta4 integrin expression in epidermis results in enhanced tumourigenesis and disruption of TGFbeta signalling. J Cell Sci 116, 3783-3791, doi:10.1242/jcs.00725 [pii] (2003).
70. Stumpfova, M., Ratner, D., Desciak, E. B., Eliezri, Y. D. & Owens, D. M. The immunosuppressive surface ligand CD200 augments the metastatic capacity of squamous cell carcinoma. Cancer Res 70, 2962-2972, doi:10.1158/0008-5472.CAN-09-4380 [pii] (2010).
71. Wheeler, D. L. et al. Protein kinase C epsilon is an endogenous photosensitizer that enhances ultraviolet radiation-induced cutaneous damage and development of squamous cell carcinomas. Cancer Res 64, 7756-7765, doi:64/21/7756 [pii] 10.1158/0008-5472.CAN-04-1881 (2004).
72. Back, J. H. et al. Resveratrol-mediated downregulation of Rictor attenuates autophagic process and suppresses UV-induced skin carcinogenesis. Photochem Photobiol 88, 1165-1172, doi:10.1111/j.1751-1097.2012.01097.x (2012).
73. Back, J. H. et al. Cancer cell survival following DNA damage-mediated premature senescence is regulated by mammalian target of rapamycin (mTOR)-dependent Inhibition of sirtuin 1. J Biol Chem 286, 19100-19108, doi:10.1074/jbc.M111.240598 [pii] (2011).
74. Jensen, U. B. et al. A distinct population of clonogenic and multipotent murine follicular keratinocytes residing in the upper isthmus. J Cell Sci 121, 609-617, doi:10.1242/jcs.025502 [pii] (2008).
75. Raj, D., Brash, D. E. & Grossman, D. Keratinocyte apoptosis in epidermal development and disease. J Invest Dermatol 126, 243-257, doi:5700008 [pii] (2006).
76. Woo, S. H., Stumpfova, M., Jensen, U. B., Lumpkin, E. A. & Owens, D. M. Identification of epidermal progenitors for the Merkel cell lineage. Development 137, 3965-3971, doi:10.1242/dev.055970 [pii] (2010).
77. Gastaldi, C. et al. miR-193b/365a cluster controls progression of epidermal squamous cell carcinoma. Carcinogenesis 35, 1110-1120, doi:10.1093/carcin/bgt490 [pii] (2014).
78. Oh, J. W., Hsi, T. C., Guerrero-Juarez, C. F., Ramos, R. & Plikus, M. V. Organotypic skin culture. J Invest Dermatol 133, e14, doi:10.1038/jid.2013.387 [pii] (2013).
79. Nelson, A. M. Calcium-Permeable Ion Channels in Epidermal Keratinocyte and Merkel-Cell Biology Ph.D. thesis, Baylor College of Medicine, (2013).
80. Haeberle, H., Bryan, L. A., Vadakkan, T. J., Dickinson, M. E. & Lumpkin, E. A. Swelling-activated Ca2+ channels trigger Ca2+ signals in Merkel cells. PLoS One 3, e1750, doi:10.1371/journal.pone.0001750 (2008).
81. Siemens, J. et al. Spider toxins activate the capsaicin receptor to produce inflammatory pain. Nature 444, 208-212, doi:nature05285 [pii] 10.1038/nature05285 (2006).
82. Piskorowski, R., Haeberle, H., Panditrao, M. V. & Lumpkin, E. A. Voltage-activated ion channels and Ca(2+)-induced Ca (2+) release shape Ca (2+) signaling in Merkel cells. Pflugers Arch 457, 197-209, doi:10.1007/s00424-008-0496-3 (2008).
83. Bhattacharya, M. R. et al. Radial stretch reveals distinct populations of mechanosensitive mammalian somatosensory neurons. Proc Natl Acad Sci USA 105, 20015-20020, doi:10.1073/pnas.0810801105 [pii] (2008).
84. Wilson, S. R. et al. The epithelial cell-derived atopic dermatitis cytokine TSLP activates neurons to induce itch. Cell 155, 285-295, doi:10.1016/j.cell.2013.08.057 S0092-8674(13)01088-X [pii] (2013).
85. Fusi, C. et al. Transient receptor potential vanilloid 4 (TRPV4) is downregulated in keratinocytes in human non-melanoma skin cancer. J Invest Dermatol 134, 2408-2417, doi:10.1038/jid.2014.145 [pii] (2014).
86. Schramek, D. et al. Direct in vivo RNAi screen unveils myosin IIa as a tumor suppressor of squamous cell carcinomas. Science 343, 309-313, doi:10.1126/science.1248627 343/6168/309 [pii] (2014).
87. Peier et al. Science (2002), 296, 2046-2049.
88. Xu et al. Nature (2002), 418, 181-185;
89. Smith et al. Nature (2002), 418, 186-188).
90. Lingam, et al., Fused Pyrimidineone Compounds as TRPV3 modulators, U.S. Ser. No. 13/348,272).

1. A method comprising:
(i) identifying a subject having a keratinocyte-derived lesion, comprising non-melanoma skin cancers and Actinic Keratosis,
(ii) administering to the lesion a therapeutically effective amount of a TRPV3 agonist, thereby treating or preventing the lesion.

2. The method of claim 1, wherein the TRPV3 agonist is camphor or 2-APB.

3. The method of claim 2, wherein the amount of camphor ranges from 4-8 mM.

4. The method of claim 2, wherein the amount of camphor ranges from 0.0608%-99.5%, or from 10%-50%.

5. The method of claim 2, wherein the amount of 2-APB ranges from 25 μM to 50 μM.

6. The method of claim 2, wherein the amount of 2-APB ranges from 0.000056-1% (wt/wt).

7. The method of claim 1, wherein the TRPV3 agonist is applied to the lesion before it is surgically removed or it is applied to the affected area from which the lesion was surgically removed, or both.

8. The method of claim 1, wherein the nonmelanoma cancer is squamous cell carcinoma.

9. A method comprising:
(i) identifying a subject having actinic keratosis or at risk of developing actinic keratosis; and
(ii) administering to an affected area a therapeutically effective amount of a TRPV3 agonist, thereby treating or preventing the actinic keratosis.

10. The method of claim 9, wherein the TRPV3 agonist is camphor or 2-APB.

11. The method of claim 10, wherein the amount of camphor ranges from 4-8 mM.

12. The method of claim 11, wherein the amount of camphor ranges from 0.0608%-99.5% or from 10%-50%.

13. The method of claim 10, wherein the amount of 2-APB ranges from 25 μM to 50 μM.

14. The method of claim 10, wherein the amount of 2-APB is from 0.000056-1% (wt/wt).

15. The method of claim 9, wherein the TRPV3 agonist is applied to the affected area before actinic keratosis is surgically removed, or after surgery to the affected area from which the actinic keratosis was surgically removed or both.

16. The method of claim 1, wherein the TRPV-3 agonist is selected from the group comprising: camphor, 2-APB, (+)-Borneol, (−)-Isopinocampheol, (−)-Fenchone, (−)-Trans-pinocarveol, Isoborneol, (+)-Camphorquinone, (−)-a-Thujone, 6-tert-butyl-m-cresol, Carvacrol, Thymol, p-xylenol, Kreosol, Propofol, Dihydrocarveol, (−)-Carveol, (−)-Isopulegol, and (+)-Linalool, or a biologically active derivative thereof.

17. The method of claim 9, wherein the TRPV-3 agonist is selected from the group comprising: camphor, 2-APB, (+)-Borneol, (−)-Isopinocampheol, (−)-Fenchone, (−)-Trans-pinocarveol, Isoborneol, (+)-Camphorquinone, (−)-a-Thujone, 6-tert-butyl-m-cresol, Carvacrol, Thymol, p-xylenol, Kreosol, Propofol, Dihydrocarveol, (−)-Carveol, (−)-Isopulegol, and (+)-Linalool, or a biologically active derivative thereof.

18. A method comprising:
(i) obtaining a biopsy of a nonmelanoma skin cancer from a subject;
(ii) obtaining a control biopsy either from a normal subject not afflicted with cancer, or a matched-sample from a non-affected area from subject;
(iii) determining the level of TRPV3 mRNA in the subject biopsy and the level of TRPV3 in the control biopsy; and
(iv) diagnosing the nonmelanoma skin cancer as a high-risk form if the level of TRPV3 mRNA in the squamous cell carcinoma biopsy is either significantly higher or significantly lower than the level in the control biopsy.

19. The method of claim 18, wherein if a diagnosis of a high-risk form of cancer is made, then determining that the subject is in need of aggressive treatment for the high-risk form of cancer.

20. The method of claim 19, wherein the aggressive treatment comprises surgery to remove the high-risk cancer in combination with application of therapeutically effective amounts of one or more TRPV3 agonists to the cancer before removal and to the affected area after it is removed.

21. The method of claim 18, wherein the nonmelanoma cancer is squamous cell carcinoma.

22. The method of claim 18, wherein the subject is an immunocompromised patient.

23. The method of claim 22 wherein the immunocompromised patient is an organ transplant patient.

24. A pharmaceutical composition comprising therapeutically effective amounts of camphor in a range of from about from 0.0608%-99.5%, or 2-APB in a range of from about 0.000056-1% or a combination of both formulated for topical application or microinjection or formulated into liposomes.

25. A pharmaceutical composition comprising therapeutically effective amounts of one or more TRPV3 agonists selected from the group comprising: camphor, 2-APB, (+)-Borneol, (−)-Isopinocampheol, (−)-Fenchone, (−)-Trans-pinocarveol, Isoborneol, (+)-Camphorquinone, (−)-a-Thujone, 6-tert-butyl-m-cresol, Carvacrol, Thymol, p-xylenol, Kreosol, Propofol, Dihydrocarveol, (−)-Carveol, (−)-Isopulegol, and (+)-Linalool or derivatives thereof.

26. The method of claim 1, wherein nonmelanoma cancer is a nonaggressive form or squamous cell carcinoma or a high-risk form of squamous cell carcinoma.

27. A sunscreen comprising one or more TRPV3 agonists selected from the group comprising camphor, 2-APB, (+)-Borneol, (−)-Isopinocampheol, (−)-Fenchone, (−)-Trans-pinocarveol, Isoborneol, (+)-Camphorquinone, (−)-a-Thujone, 6-tert-butyl-m-cresol, Carvacrol, Thymol, p-xylenol, Kreosol, Propofol, Dihydrocarveol, (−)-Carveol, (−)-Isopulegol, and (+)-Linalool or a biologically active derivative thereof.
